(19) United States
(12) Patent Application Publication
(10) Pub. No.: US 2023/0271923 A1
(43) Pub. Date: Aug. 31, 2023

(54) IMAGING AND TARGETING PROGRAMMED DEATH LIGAND-1 (PD-LI) EXPRESSION

(71) Applicant: The Johns Hopkins University, Baltimore, MD (US)

(72) Inventors: Sridhar Nimmagadda, Baltimore, MD (US); Dhiraj Kumar, Baltimore, MD (US); Martin Gilbert Pomper, Baltimore, MD (US)

(21) Appl. No.: 18/040,858

(22) PCT Filed: Aug. 6, 2021
(86) PCT No.: PCT/US2021/044951

§ 371 Date: Feb. 7, 2023

Related U.S. Application Data
(60) Provisional application No. 63/062,823, filed on Aug. 7, 2020.

(51) Int. Cl. C07D 209/04; G01N 33/534; G01N 33/543; G01N 33/569

(52) U.S. Cl. CPC C07D 209/04; G01N 33/534; G01N 33/54326; G01N 33/54346; G01N 33/56911; G01N 2800/7095

(57) ABSTRACT

The presently disclosed subject matter provides compositions, kits, and methods comprising imaging agents that can detect Programmed Cell Death Ligand 1 (PD-L1). The presently disclosed imaging agents can be used to detect diseases and disorders, such as cancer, infection, and inflammation, in a subject.

### FEDERALLY SPONSORED RESEARCH OR DEVELOPMENT

This invention was made with government support under CA236616 and CA166131 awarded by the National Institutes of Health. The government has certain rights in the invention.

### BACKGROUND

Despite the efficacious application of immune checkpoint therapy (ICT) across a broad range of cancers, only a subset of patients with terminal cancer experience remarkable clinical responses and survival. Ribas and Wolchok, 2018. The challenge facing clinicians and researchers alike is how to deliver the most effective immunotherapy to patients as quickly as possible. From the wealth of clinical trial data it is becoming increasingly evident that a single biomarker is unlikely to capture the scope and breadth of clinical responses to ICT. Havel et al., 2019. Rather, incorporation of multiple biomarker panels, including both pharmacodynamic and predictive biomarkers, has become a necessity. Havel et al., 2019. The number of tests that can be performed with baseline and on-treatment biopsies is limited by the amount of biopsy tissue, and has several shortcomings, including inter- and intra-tumoral heterogeneity and sampling errors. Those problems are compounded in difficult-to-access locations as in the case of lung and pancreatic cancers and limit our ability to measure pharmacodynamic effects of ICT. Imaging methods, such as positron emission tomography (PET), enable repetitive sampling of the whole body and facilitate real-time quantification of pharmacodynamic effects. PET, however, is underutilized in guiding ICT primarily due to the limited access to molecularly-targeted radiotracers that accurately report on the activity of immune infiltrate.

### SUMMARY

In some aspects, the presently disclosed subject matter provides an imaging agent comprising a compound of formula (I):

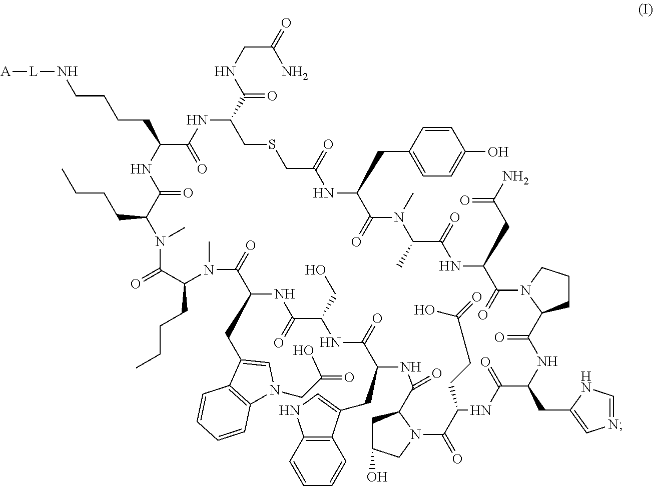

wherein: L is a linker, which can be present or absent, and when present has the following general formula:

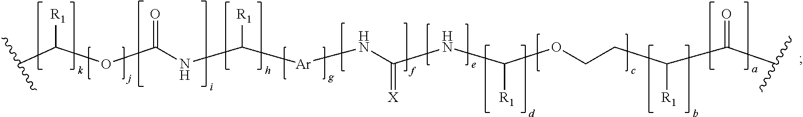

wherein: X is S or 0; a, e, f, g, i, and j are each independently an integer selected from the group consisting of 0 and 1; b, d, h, and k are each independently an integer selected from the group consisting of 0, 1, 2, 3, 4, 5, 6, 7, and 8; c is an integer having a range from 0 to 40; each R₁ is H or —COOR₂, wherein R₂ is H or C₁-C₄ alkyl; Ar is substituted or unsubstituted aryl or heteroaryl; and A is a reporting moiety selected from the group consisting of a chelating agent, a radiolabeled substrate, a fluorescent dye, a photoacoustic reporting molecule, and a Raman-active reporting molecule or an end group selected from the group consisting of —NR₃R₄ or C≡N, wherein R₃ and R₄ are each independently selected from the group consisting of H and C₁-C₄ alkyl.

In other aspects, the presently disclosed subject matter provides an imaging method for detecting Programmed Death Ligand 1 (PD-L1), the method comprising: (a) providing an effective amount of an imaging agent of formula (I); (b) contacting one or more cells or tissues with the imaging agent; and (c) making an image to detect PD-L1.

In other aspects, the presently disclosed subject matter provides a kit for detecting Programmed Death Ligand 1 (PD-L1), the kit comprising the imaging agent of formula (I).

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

This patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1A, FIG. 1B, FIG. 1C, and FIG. 1D show the synthesis and in vitro characterization of [¹⁸F]DK222. FIG. 1A shows the structure and schema for the preparation of [¹⁸F]DK222. FIG. 1B demonstrates that DK221, DK222 and the non-radioactive [¹⁹F]DK222 inhibit PD1:PD-L1 interaction at nanomolar concentrations in a protein-based assay. FIG. 1C is flow cytometry histograms showing graded level of PD-L1 expression in human TNBC, melanoma and Chinese Hamster Ovarian cells with stable human PD-L1 expression. FIG. 1D demonstrates that [¹⁸F]DK222 binding to cells is PD-L1 expression dependent and reduced in the presence of 1 μM unmodified peptide demonstrating specificity. ****, P<0.0001; NS, not significant, by unpaired t-test in FIG. 1D;

FIG. 2A, FIG. 2B, FIG. 2C, and FIG. 2D show in vivo kinetics of [¹⁸F]DK222 in mice bearing TNBC xenografts. FIG. 2A demonstrates that high and specific uptake of [¹⁸F]DK222 can be seen in high PD-L1 expressing MDAMB231 tumors, but not in mice receiving blocking dose or low PD-L1 expressing SUM149 tumors (n=3-4). Whole body volume rendered PET-CT images of xenograft bearing NSG mice acquired at 15, 60 and 120 min after 200 mCi (7.4 MBq) of [¹⁸F]DK222 injection. Blocking dose mice received 50 mg/kg of unmodified peptide 30 mn prior to radiotracer injection. FIG. 2B shows that [¹⁸F]DK222 exhibits rapid accumulation and retention in MDAMB231 tumors for several hours that is not observed with SUM149 tumors or with the blocking dose (n=4-5). FIG. 2C is time-activity curves derived from biodistribution data show rapid clearance of [¹⁸F]DK222 from circulation as indicated by high target-to-muscle (and blood) ratios (n=4-5). Data are derived from biodistribution studies shown in Table 1. FIG. 2D shows IHC staining for PD-L1 of the corresponding tumors. ****, P<0.0001; NS, not significant, by unpaired t-test in FIG. 2C;

FIG. 3A, FIG. 3B, and FIG. 3C illustrate that [¹⁸F]DK222 PET in mice with human melanoma xenografts shows high contrast images at 60 min. FIG. 3A shows high and specific uptake of [¹⁸F]DK222 in LOX-IMVI tumors that express PD-L1 and not in mice receiving blocking dose or low PD-L1 expressing MeWo tumors (n=3-4). Whole body volume rendered PET-CT images of xenograft bearing mice acquired at 60 min after [¹⁸F]DK222 injection. FIG. 3B shows tumor uptake of [¹⁸F]DK222 by ex vivo biodistribution in NSG mice bearing LOX-IMVI or MeWo tumors (n=5). FIG. 3C is IHC staining for PD-L1 of the corresponding tumors. ****, P<0.0001; NS, not significant, by unpaired t-test in FIG. 3B;

FIG. 4A, FIG. 4B, FIG. 4C, FIG. 4D, FIG. 4E, FIG. 4F, and FIG. 4G demonstrate that [¹⁸F]DK222 uptake correlates with total PD-L1 levels in the tumors induced by aPD-1 therapeutics. FIG. 4A is an experimental schematic. FIG. 4B demonstrates that huPBMC mice with A375 melanoma tumors and treated with a single dose of 10 mg/kg of Nivolumab or Pembrolizumab for 7 days show increased [¹⁸F]DK222 uptake in the tumors. Representative images of 3 mice are shown in FIG. 4B and FIG. 4C. FIG. 4C illustrates that IHC analysis of tumor sections from imaging mice show increased immunoreactivity for PD-L1 and CD3 in Nivolumab and Pembrolizumab treated mice compared to saline treated controls and NSG mice. FIG. 4D is [¹⁸F]DK222 uptake in tumors quantified by biodistribution (n=8-13). FIG. 4E and FIG. 4F demonstrate that PD-L1 levels on tumor and immune cells (FIG. 4E) and number of CD45 cells analyzed by flow cytometry (FIG. 4F) show the effects of different PD-1 antibodies. FIG. 4G illustrates that a strong correlation is observed between [¹⁸F]DK222 uptake and total PD-L1 levels in the tumor microenvironment. ****P<0.0001; ***, P<0.001; **, P<0.01 by 1-way ANOVA in FIG. 4D. Simple linear regression and Pearson coefficient in FIG. 4G with 95% CI;

FIG. 5A, FIG. 5B, FIG. 5C, FIG. 5D, and FIG. 5E demonstrate that accessible PD-L1 levels quantified using [¹⁸F]DK222 show a dose dependent PD-L1 engagement by Atezolizumab. FIG. 5A demonstrates that [¹⁸F]DK222 allows quantification of accessible PD-L1 levels in vitro in the presence of aPD-L1 mAbs; FIG. 5B is an experimental schematic. FIG. 5C and FIG. 5E demonstrate that reduced [¹⁸F]DK222 uptake is observed in the LOX-IMVI tumors with increased Atezolizumab dose. NSG mice were treated with different doses of Atezolizumab for 24 hours prior to the [¹⁸F]DK222 injection. Whole body volume rendered PET-CT images of mice acquired at 60 after [¹⁸F]DK222 injection (FIG. 5D) (n=3), and ex vivo biodistributions (FIG. 5E) (n=5). ****, P<0.0001, **, P<0.01 by 1-way ANOVA and Tukey's multiple comparisons test in FIG. 5E;

FIG. 6A, FIG. 6B, FIG. 6C, and FIG. 6D show the pharmacologic activity of PD-L1 therapeutics quantified at the tumor using [¹⁸F]DK222-PET. FIG. 6A is an experimental schematic. FIG. 6B shows that [¹⁸F]DK222 uptake in LOX-IMVI tumors in mice treated with 1 mg/kg of antibodies for 24 and 96 hrs captures differing PD-L1 occupancy and PK at the tumor that is antibody affinity dependent (n=3). NSG mice were treated with Atezolizumab, Avelumab or Durvalumab at 1 mg/kg dose for 24 and 96 hours prior to the [¹⁸F]DK222 injection. Nivolumab at 1 mg/kg and saline are used as controls. Whole body volume rendered PET/CT images of mice acquired at 60 min after [¹⁸F]DK222 injection. FIG. 6C and FIG. 6D show [¹⁸F]DK222 uptake in tumors quantified by biodistribution in LOX-IMVI (FIG. 6D, n=8-19) and MDAMB231 (FIG. 6D, n=7-18) tumor bearing mice;

FIG. 7A shows [¹⁸F]DK222 PET in a non-human primate (Papio anubus). Papio Anubis was injected with ˜5 mCi of PET images of [¹⁸F]DK222 and whole-body images were acquired at different time points. PET images showed major radioactivity uptake in bladder, kidneys and spleen. Interestingly, high uptake also is observed in what are likely lymph nodes;

FIG. 8A, FIG. 8B, FIG. 8C, and FIG. 8D demonstrate that [¹⁸F]DK222 PET in mice with human lung cancer xenografts shows high contrast images at 60 min. FIG. 8A is PD-L1 expression levels in lung cancers analyzed by flow cytometry. FIG. 8B is in vitro uptake of [¹⁸F]DK222 in lung cancer cell lines. FIG. 8C show high and specific uptake of [¹⁸F]DK222 in H2444 tumors that express PD-L1 and not in mice with low PD-L1 expressing A549 tumors (n=3-4). Whole body volume rendered PET-CT images of xenograft bearing mice acquired at 60 min after [¹⁸F]DK222 injection. FIG. 8D is tumor uptake of [¹⁸F]DK222 by ex vivo biodistribution in NSG mice bearing H2444, H226 and A549 tumors (n=5);

FIG. 9A, FIG. 9B, FIG. 9C, and FIG. 9D demonstrate that [¹⁸F]DK222 PET in mice with human bladder cancer xenografts shows high contrast images at 60 min. FIG. 9A is PD-L1 expression levels in bladder cancer cell lines analyzed by flow cytometry. FIG. 9B is in vitro uptake of [¹⁸F]DK222 in bladder cancer cells with variable PD-L1 expression. FIG. 9C shows high and specific uptake of [¹⁸F]DK222 in BFTC909 tumors that express PD-L1 and not in mice with low PD-L1 expressing SCaBer tumors (n=3-4). Whole body volume rendered PET-CT images of xenograft bearing mice acquired at 60 min after [¹⁸F]DK222 injection. FIG. 9D shows tumor uptake of [¹⁸F]DK222 by ex vivo biodistribution in NSG mice bearing BFTC909, T24 and SCaBER tumors (n=5);

FIG. 10 is the structure of DK221 and a schematic for the synthesis of [¹⁹F]DK222;

FIG. 11A, FIG. 11B, FIG. 11C, and FIG. 11D are: FIG. 11A, reverse phase HPLC chromatogram of DK222. FIG. 11B, ESI-MS of DK222. FIG. 11C, Reverse phase HPLC chromatogram of [¹⁹F]DK222. FIG. 11D, ESI-MS of [¹⁹F]DK222;

FIG. 12 is a schematic for the synthesis of [¹⁸F]DK222;

FIG. 13A, FIG. 13B, and FIG. 13C are: FIG. 13, reverse phase HPLC chromatogram of crude reaction mixture of [¹⁸F]DK222. FIG. 13B, radiochemical purity of [¹⁸F]DK222. FIG. 13C, Chemical identity of [¹⁸F]DK222;

FIG. 14 shows the stability of formulated [¹⁸F]DK222;

FIG. 15A, FIG. 15B, and FIG. 15C show: FIG. 15A, effect of non-radioactive DK221 carrier on [¹⁸F]DK222 uptake in MDAMB231 and SUM149 tumors. Co-injection of variable amounts of DK221 with [¹⁸F]DK222 shows reduction in radioactivity uptake with increased carrier dose in PD-L1 positive MDAMB231 tumors but not in PD-L1 negative SUM149 tumors. FIG. 15B, biodistribution data showing mean % ID/g values with 95% confidence intervals. FIG. 15C, Carrier dose has minimal effect on [¹⁸F]DK222 uptake in selective tissues. The uptake in 30 μg dose group is consistently high in all the tissues for reasons unknown;

FIG. 16 shows ex vivo biodistribution of [¹⁸F]DK222 in mice bearing LOX-IMVI and MEWO melanoma tumor xenografts. Mice received 50 μCi of [¹⁸F]DK222 and tissues were harvested 60 min later. Data shown is mean±SEM (n=4-5/group). **** P<0.0001 by unpaired t-test;

FIG. 17 shows the effect of IFNγ treatment on PD-L1 levels assessed by flow cytometry in melanoma cell lines;

FIG. 18 shows the selected tissue ex vivo biodistribution of [¹⁸F]DK222 in huPBMC mice bearing A375 xenografts and treated with aPD-1 mAbs. Mice received 50 μCi of [¹⁸F]DK222 and tissues were harvested 60 min later;

FIG. 19 shows selected tissue ex vivo biodistribution of [¹⁸F]DK222 in NSG mice bearing LOX-IMVI xenografts and treated with 0.3 mg/kg and 20 mg/kg dose of Atezolizumab. Mice received 50 μCi of [¹⁸F]DK222 and tissues were harvested 60 min later;

FIG. 20 shows selected tissue ex vivo biodistribution of [¹⁸F]DK222 in NSG mice bearing LOX-IMVI xenografts and treated with 1 mg/kg dose of aPD-L1 mAbs for 24 and 96 h;

FIG. 21 is the MALDI-TOF MS of DK222;

FIG. 22 is the ESI-MS of DK331;

FIG. 23 is the MALDI-MS of DK331;

FIG. 24 is the ESI-MS of DK225;

FIG. 25 is the MALDI-MS of DK223;

FIG. 26 is the MALDI-MS of DK385;

FIG. 27 is the ESI-MS of DK254;

FIG. 28 is the ESI-MS of DK265;

FIG. 29 is the ESI-MS of DK365;

FIG. 30 is the ESI-MS of DK360;

FIG. 31 is the MALDI-TOF of DK388;

FIG. 32 is the RP-HPLC of crude [¹⁸F]PyTFP;

FIG. 33 is the RP-HPLC of crude [¹⁸F]DK221Py;

FIG. 34 is the RP-HPLC of pure [¹⁸F]DK221Py;

FIG. 35 is the in vivo evaluation of [¹⁸F]DK221Py in hPD-L1/CHO;

FIG. 36 shows data from an HTRF PD1/PD-L1 binding assay for DK221, DK222, and DK291 ([¹⁹F]DK222); and

FIG. 37 shows data from an HTRF PD1/PD-L1 binding assay for DK225, DK223, DK385, and DK331.

### DETAILED DESCRIPTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Imaging and Targeting Programmed Death Ligand-1 (Pd-L1) Expression

Current tools to quantify immune responses in the whole body are limited. The presently disclosed subject matter, in part, is directed to the development of a radiopharmaceutical for the most widely used biomarker, i.e., programmed death ligand-1 (PD-L1), for selecting patients for immune checkpoint therapy (ICT) and has proven useful in predicting response to ICT in several cancers. Garon et al., 2015; Reck et al., 2016; Reck et al., 2019; Herbst et al., 2019; Hellmann et al., 2018; Peters et al., 2019; Spigel et al., 2019; Yarchoan et al., 2019; Melosky et al., 2018. To this end, a peptide-based radiopharmaceutical and analogs were developed for measuring PD-L1 levels to predict ICT efficacy in real-time.

More particularly, the presently disclosed subject matter provides, in part, a highly specific peptide-based positron emission tomography (PET) imaging agent capable of detecting PD-L1 expression in tumors and immune cells soon after injection of the radiotracer. The presently disclosed imaging agent fits within the standard clinical workflow of imaging within 60 min of administration and are applicable for imaging various types of cancers, infectious and inflammatory entities including, but not limited to, experimental models of chronic bacterial infection, disseminated tuberculosis, lupus, and rheumatoid arthritis.

### A. Compositions Comprising Imaging Agents

In some embodiments, the presently disclosed subject matter provides an imaging agent comprising a compound of formula (I):

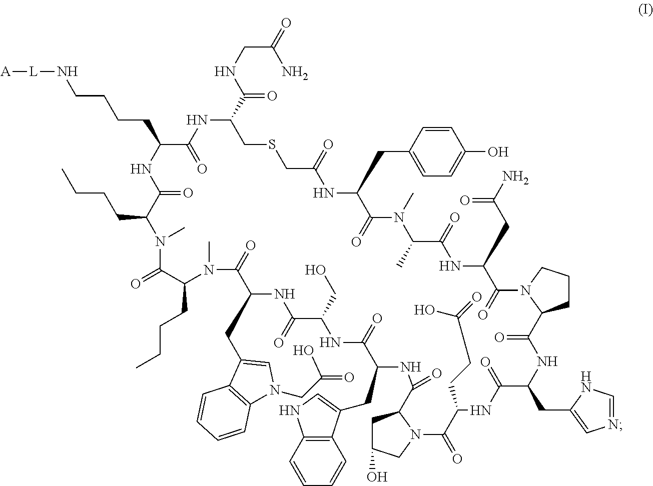

wherein: L is a linker, which can be present or absent, and when present has the following general formula:

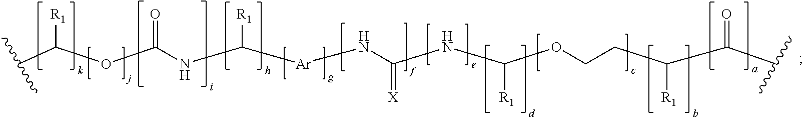

wherein: X is S or 0; a, e, f, g, i, and j re each independently integers selected the group consisting of 0 and 1; b, d, h, and k are each independently an integer selected from the group consisting of 0, 1, 2, 3, 4, 5, 6, 7, and 8; c is an integer having a range from 0 to 40; each R₁ is H or —COOR₂, wherein R₂ is H or C₁-C₄ alkyl; Ar is substituted or unsubstituted aryl or heteroaryl; and A is a reporting moiety selected from the group consisting of a chelating agent, a radiolabeled substrate, a fluorescent dye, a photoacoustic reporting molecule, and a Raman-active reporting molecule or an end group selected from the group consisting of —NR₃R₄ or C≡N, wherein R₃ and R₄ are each independently selected from the group consisting of H and C₁-C₄ alkyl.

As used herein, C₁-C₄ alkyl include methyl, ethyl, n-propyl, isopropyl, n-butyl, isobutyl, sec-butyl, and tert-butyl. The term “aryl” means, unless otherwise stated, an aromatic hydrocarbon substituent that can be a single ring or multiple rings (such as from 1 to 3 rings), which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized.

In some embodiments, the linker is selected from the group consisting of:

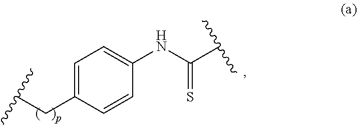

wherein p is an integer selected from 0, 1, 2, 3, and 4;

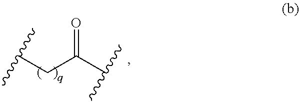

wherein q is an integer selected from the group consisting of 0, 1, 2, 3, 4, 5, 6, 7, and 8;

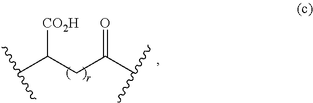

wherein r is an integer selected from the group consisting of 0, 1, 2, 3, 4, 5, 6, 7, and 8;

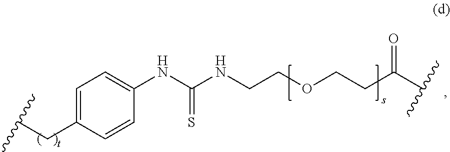

wherein s is an integer having a range from 1 to 40 and t is an integer selected from 0 or 1;

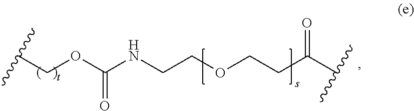

wherein s is an integer having a range from 1 to 40 and t is an integer selected from 0 or 1; and

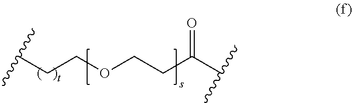

wherein s is an integer having a range from 1 to 40 and t is an integer selected from 0 or 1.

One of ordinary skill in the art would recognize upon review of the presently disclosed subject matter that a variety of combinations of chelating agents/radiometal ions are suitable for use with the presently disclosed imaging agents. Representative chelating agents are known in the art. By way of non-limiting examples, certain chelating agents and linkers are disclosed in U.S. patent application publication numbers 2015/0246144 and 2015/0104387, each of which is incorporated herein by reference in their entirety.

In some embodiments, the reporting moiety is a chelating agent and the chelating agent is selected from the group consisting of DOTAGA (1,4,7,10-tetraazacyclododececane, 1-(glutaric acid)-4,7,10-triacetic acid), DOTA (1,4,7,10-tetraazacyclododecane-1,4,7,10-tetraacetic acid), DOTA-tris(t-butyl)ester, DOTAGA-(t-butyl)₄, DOTA-di(t-butyl)ester, DOTASA (1,4,7,10-tetraazacyclododecane-1-(2-succinic acid)-4,7,10-triacetic acid), CB-DO2A (10-bis(carboxymethyl)-1,4,7,10-tetraazabicyclo[5.5.2]tetradecane), DEPA (7-[2-(Bis-carboxymethylamino)-ethyl]-4,10-bis-carboxymethyl-1,4,7,10-tetraaza-cyclododec-1-yl-acetic acid)), 3p-C-DEPA (2-[(carboxymethyl)][5-(4-nitrophenyl-1-[4,7,10-tris(carboxymethyl)-1,4,7,10-tetraazacyclododecan-1-yl]pentan-2-yl)amino]acetic acid)), TCMC (2-(4-isothiocyanotobenzyl)-1,4,7,10-tetraaza-1,4,7,10-tetra-(2-carbamonyl methyl)-cyclododecane), oxo-DO3A (1-oxa-4,7,10-triazacyclododecane-5-S-(4-isothiocyanatobenzyl)-4,7,10-triacetic acid), DO3A-(t-butyl), DO3AM (2,2′,2″-(1,4,7,10-tetraazacyclododecane-1,4,7-triyl)triacetamide), p-NH2-Bn-Oxo-DO3A (1-Oxa-4,7,10-tetraazacyclododecane-5-S-(4-aminobenzyl)-4,7,10-triacetic acid), TE2A ((1,8-N,N-bis-(carboxymethyl)-1,4,8,11-tetraazacyclotetradecane), MM-TE2A, DM-TE2A, CB-TE2A (4,11-bis(carboxymethyl)-1,4,8,11-tetraazabicyclo[6.6.2]hexadecane), CB-TE1A1P (4,8,11-tetraazacyclotetradecane-1-(methanephosphonic acid)-8-(methanecarboxylic acid), CB-TE2P (1,4,8,11-tetraazacyclotetradecane-1,8-bis(methanephosphonic acid), TETA (1,4,8,11-tetraazacyclotetradecane-1,4,8,11-tetraacetic acid), NOTA (1,4,7-triazacyclononane-N,N′,N″-triacetic acid), NOTA(t-butyl)₂, NO2A (1,4,7-Triazacyclononane-1,4-bis(acetic acid)-7-(acetamide), NODA (1,4,7-triazacyclononane-1,4-diacetate); NODAGA (1,4,7-triazacyclononane, 1-glutaric acid-4,7-acetic acid), NODAGA(t-butyl)₃, NOTAGA (1,4,7-triazonane-1,4-diyl)diacetic acid), DFO (Desferoxamine), DTPA (2-[Bis[2-[bis(carboxymethyl)amino]ethyl]amino]acetic acid), DTPA-tetra(t-butyl)ester (diethylenetriamine-N,N,N″,N″-tetra-tert-butyl acetate-N′-acetic acid), NETA ([4-[2-(bis-carboxymethylamino)-ethyl]-7-carboxymethl-[1,4,7]triazonan-1-yl}-acetic acid), TACN-TM (N,N′,N″, tris(2-mercaptoethyl)-1,4,7-triazacyclononane), Diamsar (1,8-Diamino-3,6,10,13,16,19-hexaazabicyclo(6,6,6)eicosane, 3,6,10,13,16,19-Hexaazabicyclo[6.6.6]eicosane-1,8-diamine), Sarar (1-N-(4-aminobenzyl)-3,6,10,13,16,19-hexaazabicyclo[6.6.6] eicosane-1,8-diamine), AmBaSar (4-((8-amino-3,6,10,13,16,19-hexaazabicyclo [6.6.6] icosane-1-ylamino) methyl) benzoic acid), BaBaSar, tris(hydroxypyridinone) (THP), THP(benzyl)₃, NOPO (3-(((4,7-bis((hydroxy(hydroxymethyl)phosphoryl)-methyl)-1,4,7-triazonan-1-yl)methyl)(hydroxy)phosphoryl)propanoic acid), TRAP (3,3′,3″-(((1,4,7-triazonane-1,4,7-triyl)tris(methylene))tris(hydroxyphosphoryl))-tripropanoic acid), p-NH₂—Bn-PCTA (3,6,9,15-Tetraazabicyclo[9.3.1] pentadeca-1(15),11,13-triene-4-S-(4-aminobenzyl)-3,6,9-triacetic acid), and biotin (5-[(3aS,4S,6aR)-2-oxohexahydro-TH-thieno[3,4-d]imidazol-4-yl]pentanoic acid).

In some embodiments, the chelating agent is selected from the group consisting of

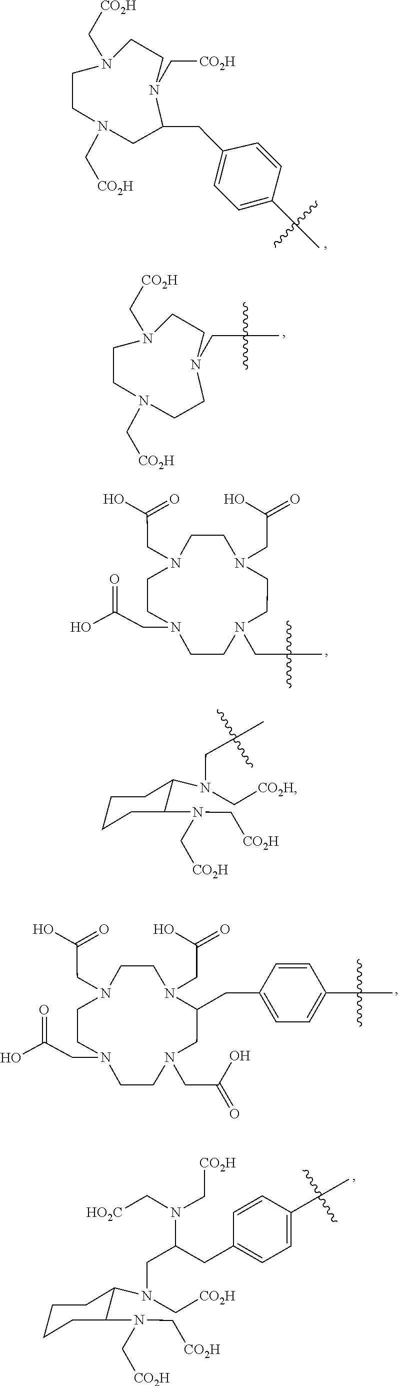

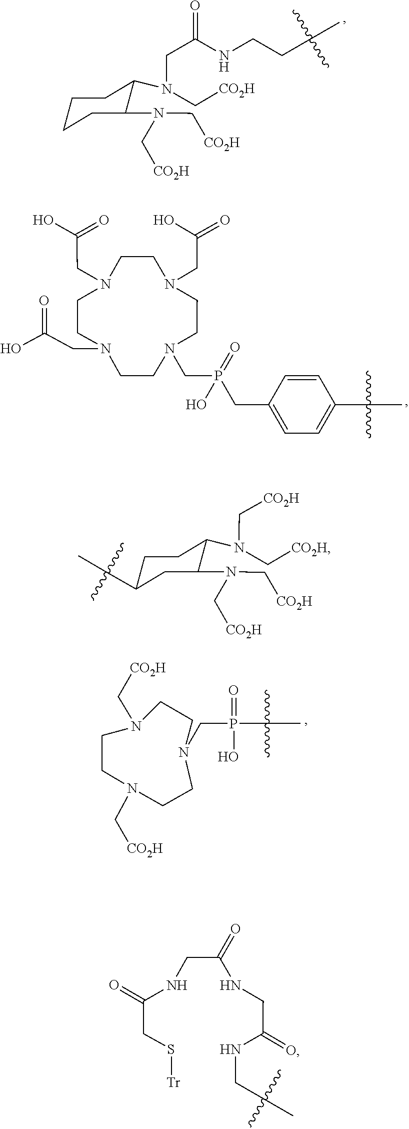

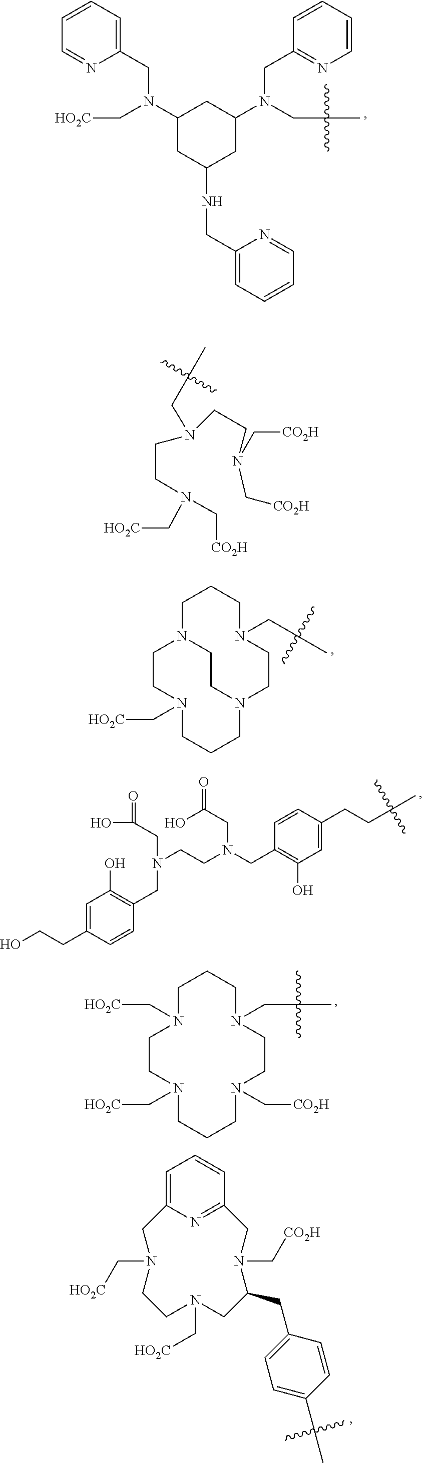

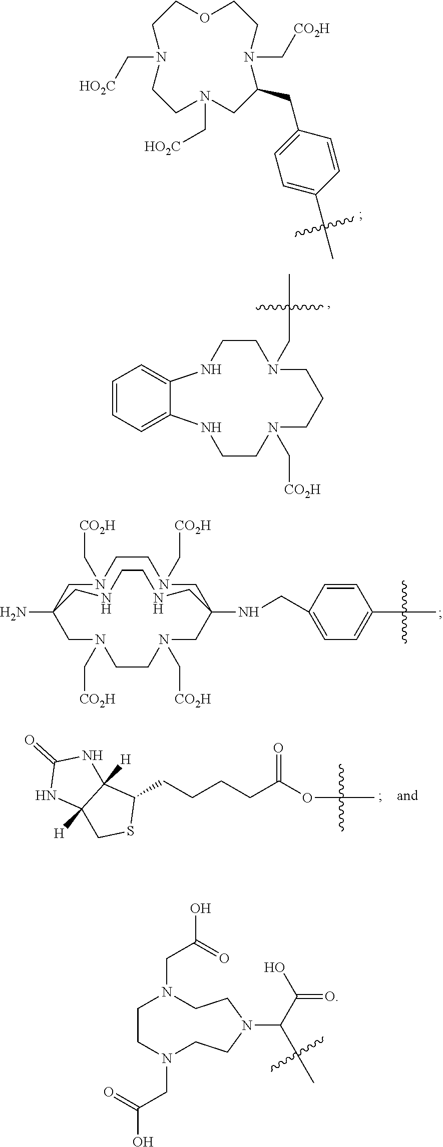 [SMILES: *c1ccc(C[C@H]2CN(CC(=O)O)CCN(CC(=O)O)CCOCCN2CC(=O)O)cc1 ; *CN1CCCN(CC(=O)O)CCNc2ccccc2NCC1 ; *c1ccc(CNC23CNCCNCC(N)(CN(CC(=O)O)CCN(CC(=O)O)C2)CN(CC(=O)O)CCN(CC(=O)O)C3)cc1 ; *OC(=O)CCCC[C@@H]1SC[C@@H]2NC(=O)N[C@@H]21 ; *C(C(=O)O)N1CCN(CC(=O)O)CCN(CC(=O)O)CC1]

In some embodiments, the chelating agent is selected from the group consisting of DOTA (1,4,7,10-tetraazacyclododecane-1,4,7,10-tetraacetic acid), NOTA (1,4,7-triazacyclononane-N,N′,N″-triacetic acid), NODA (1,4,7-triazacyclononane-1,4-diacetate); NODAGA (1,4,7-triazacyclononane, 1-glutaric acid-4,7-acetic acid), and biotin (5-[(3aS,4S,6aR)-2-oxohexahydro-1H-thieno[3,4-d]imidazol-4-yl]pentanoic acid).

In some embodiments, the reporting moiety is a chelating agent and the chelating agent further comprises a radiometal selected from the group consisting of ^{94m}Tc, ^{99m}Tc, ¹¹¹In, ⁶⁷Ga ⁶⁸Ga, ⁸⁶Y, ⁹⁰Y, ¹⁷⁷Lu, ¹⁸⁶Re, ¹⁸⁸Re, ⁶⁰Cu, ⁶¹Cu, ⁶²Cu, ⁶⁴Cu, ⁶⁷Cu, ⁵⁵Co, ⁵⁷Co, ⁴⁴Sc, ⁴⁷Sc, ²²⁵Ac, ²¹³Bi, ²¹²Bi, ²¹²Pb, ¹⁵³Sm, ¹⁶⁶Ho, ¹⁵²Gd, ⁸²Rb, ⁸⁹Zr, ¹⁶⁶Dy, and Al¹⁸F.

In some embodiments, the substrate is labeled with ¹⁸F using the AlF method, for example, based on the chelation of aluminum fluoride by NOTA, NODA, or any other suitable chelator known in the art. See, for example, Liu S., et al., “One-step radiosynthesis of ¹⁸F-AlF-NOTA-RGD₂ for tumor angiogenesis PET imaging. Eur J Nucl Med Mol Imaging. 2011, 38(9):1732-41; McBride W. J., et al., “A novel method of ¹⁸F radiolabeling for PET. J Nucl Med. 2009; 50:991-998; McBride W. J, D'Souza C A, Sharkey R M, Sharkey R M, Karacay H, Rossi E A, Chang C-H, Goldenberg D M. Improved ¹⁸F labeling of peptides with a fluoride-aluminum-chelate complex. Bioconjug Chem. 2010; 21:1331-1340.

One of ordinary skill in the art would recognize that, in some embodiments, the linker, “L,” of formula (I) is absent and the chelating agent is conjugated with DK221 through a linker moiety that is part of the chelating agent as supplied. For example, as provided in Example 1 herein below, in particular embodiments, the lysine s-amine of DK221 is used for bifunctional chelator conjugation using the NHS ester method. For example, if the chelating agent as supplied is NCS-MP-NODA (2,2′-(7-(4-isothiocyanatobenzyl)-1,4,7-triazonane-1,4-diyl)diacetic acid), the isothiocyanatobenyzl moiety is the linker between the NODA chelating agent and the lysine ε-amine of DK221.

Other linker moieties that can comprise the chelating agent as supplied include, but are not limited to, maleimide, NHS ester, anhydride, NCS, NCS-benzyl, NH₂-PEG, BCN, —NH₂, propargyl, acetic acid, glutamic acid, and the like.

In some embodiments, the reporting moiety is a radiolabeled substrate and the radiolabeled substrate comprises a radioisotope selected from the group consisting of ¹¹C, ¹³N, ¹⁵O, ¹²³I, ¹²⁴I, ¹²⁵I, ¹²⁶I, ¹³¹I, ⁷⁵Br, ⁷⁶Br, ⁷⁷Br, ⁸⁰Br, ^{80m}Br, ⁸²Br, ⁸³Br, ¹⁹F, ¹⁸F, and ²¹¹At.

In some embodiments the radiolabeled substrate comprises an ¹⁸F-labeled substrate or an ¹⁸F-labeled substrate.

In some embodiments, the ¹⁹F-labeled substrate or the ¹⁸F-labeled substrate is selected from the group consisting of 2-fluoro-PABA, 3-fluoro-PABA, 2-fluoro-mannitol, and N-succinimidyl-4-fluorobenzoate, and 2-pyridyl.

In some embodiments, the reporting moiety is a fluorescent dye and the fluorescent dye is selected from the group consisting of carbocyanine, indocarbocyanine, oxacarbocyanine, thuicarbocyanine, merocyanine, polymethine, coumarine, aminomethylcoumarin acetate (AMCA), rhodamine, tetramethylrhodamine (TRITC), xanthene, fluorescein, FITC, a boron-dipyrromethane (BODIPY) dye, Cy3, Cy5, Cy5.5, Cy7, VivoTag-680, VivoTag-S680, VivoTag-S750, AlexaFluor350, AlexaFluor405, AlexaFluor488, AlexaFluor546, AlexaFluor555, AlexaFluor594, AlexaFluor633, AlexaFluor647, AlexaFluor660, AlexaFluor680, AlexaFluor700, AlexaFluor750, AlexaFluor790, Dy677, Dy676, Dy682, Dy752, Dy780, DyLight350, DyLight405, DyLight488, DyLight547, DyLight550, DyLight594, DyLight633, DyLight647, DyLight650, DyLight680, DyLight755, DyLight800, HiLyte Fluor 647, HiLyte Fluor 680, HiLyte Fluor 750, IR Dye 800, IRDye 800CW, IRDye 800RS, IRDye 700DX, ADS780WS, ADS830WS, ADS832WS, Cascade Blue, and Texas Red.

In some embodiments, the reporting moiety is a photoacoustic reporting molecule and the photoacoustic reporting molecule is selected from the group consisting of a dye or a nanoparticle.

In some embodiments, the dye comprises a fluorescent dye. In some embodiments, the fluorescent dye is selected from the group consisting of indocyanine-green (ICG), Alexa Fluor 750, Evans Blue, BHQ3, QXL680, IRDye880CW, MMPSense 680, Methylene Blue, PPCy-C8, and Cypate-C18.

In some embodiments, the nanoparticle is selected from the group consisting of a plasmonic nanoparticle, a quantum dot, a nanodiamond, a polypyrrole nanoparticle, a copper sulfide nanoparticle, a graphene nanosheet, an iron oxide-gold core-shell nanoparticle, a Gd203 nanoparticle, a single-walled carbon nanotube, a dye-loaded perfluorocarbon nanoparticle, and a superparamagnetic iron oxide nanoparticle.

In some embodiments, the reporting moiety is a Raman-active reporting molecule and the Raman-active reporting molecule is selected from the group consisting of a single-walled carbon nanotube (SWNT) and a surface-enhanced Raman scattering (SERS) agent.

In some embodiments, the SERS agent comprises a metal nanoparticle labeled with a Raman-active reporter molecule.

In some embodiments, the Raman-active reporter molecule comprises a fluorescent dye. In some embodiments, the fluorescent dye is selected from the group consisting of Cy3, Cy5, rhodamine, and a chalcogenopyrylium dye.

In some embodiments, the imaging agent of formula (I) is selected from the group consisting of:

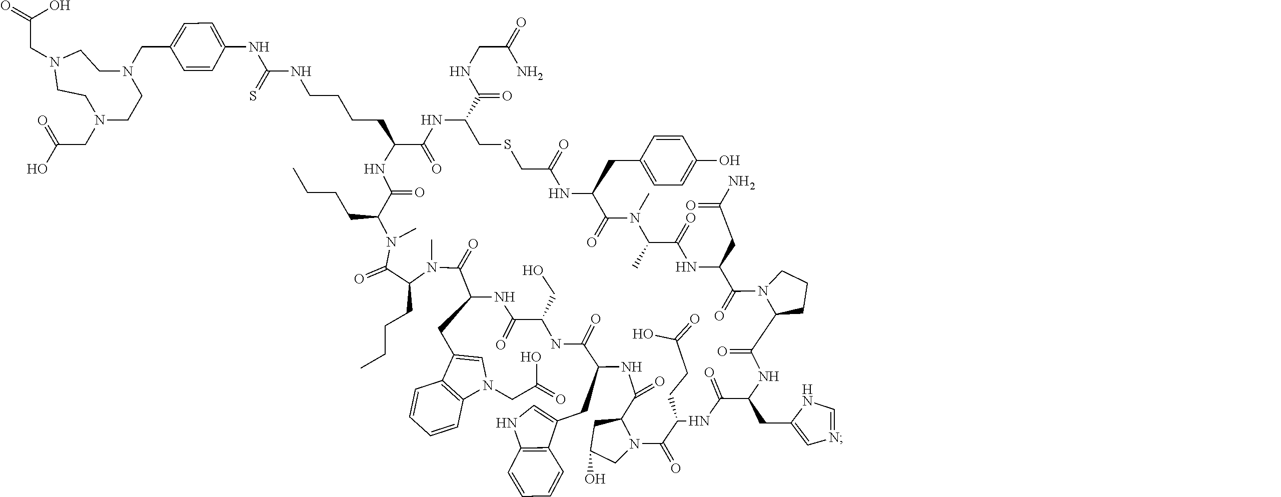

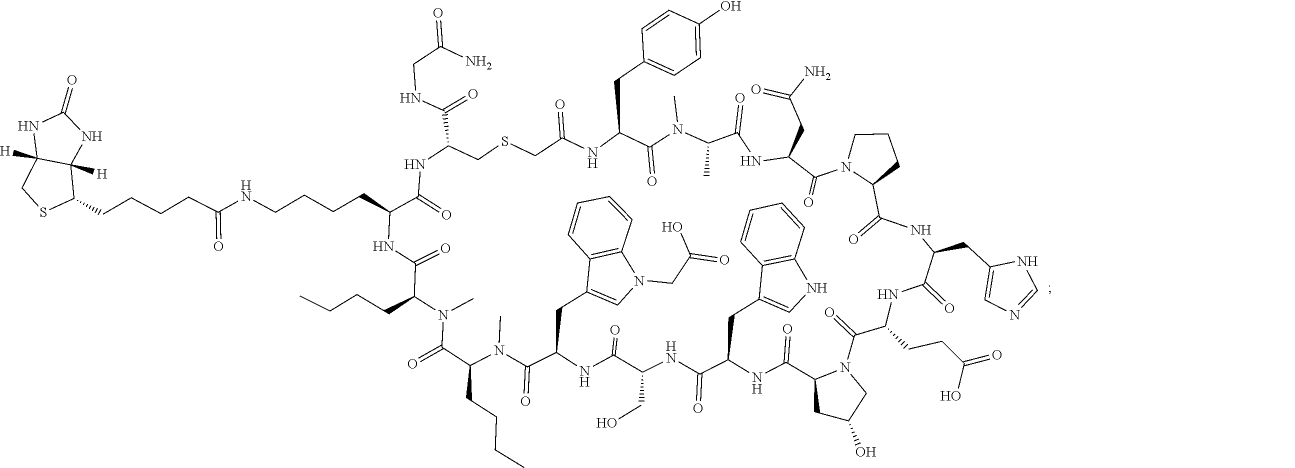 [SMILES: CCCC[C@H]1C(=O)N(C)[C@@H](CCCC)C(=O)N[C@@H](CCCCNC(=O)CCCC[C@@H]2SC[C@@H]3NC(=O)N[C@@H]32)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](Cc2cnc[nH]2)C(=O)N[C@H](CCC(=O)O)C(=O)N2C[C@H](O)C[C@H]2C(=O)N[C@H](Cc2c[nH]c3ccccc23)C(=O)N[C@H](CO)C(=O)N[C@H](Cc2cn(CC(=O)O)c3ccccc23)C(=O)N1C]

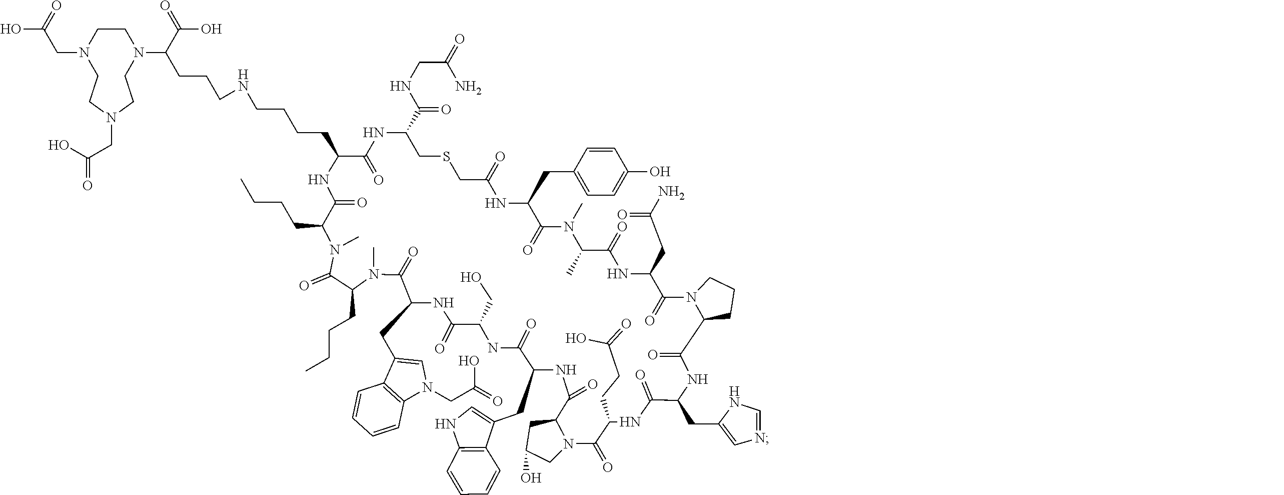

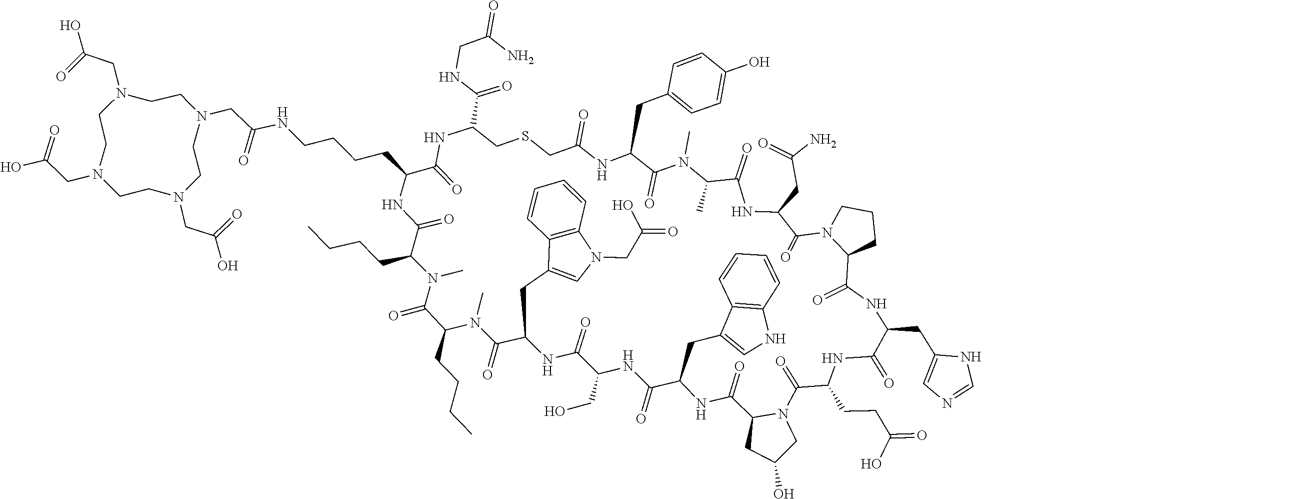

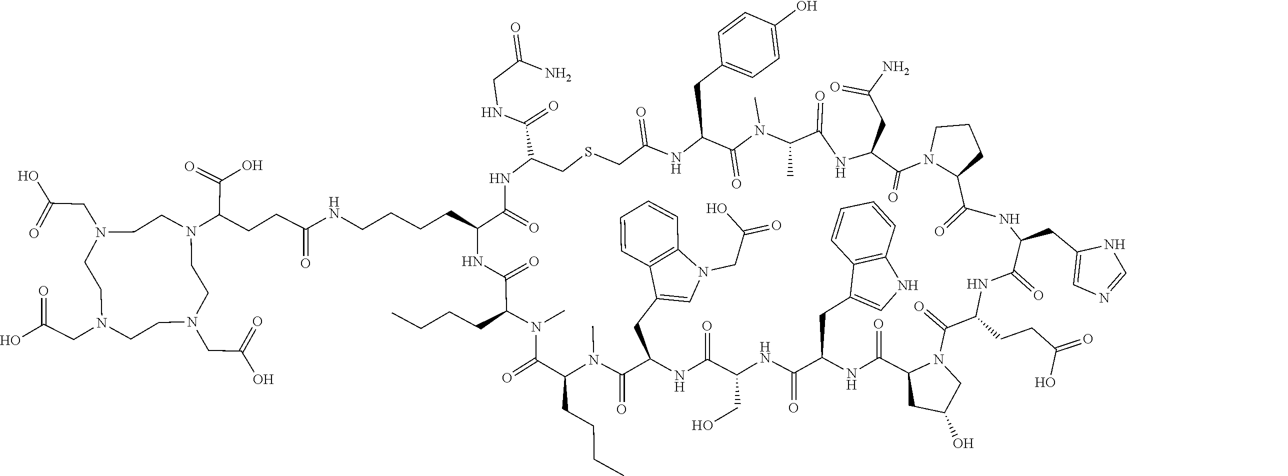

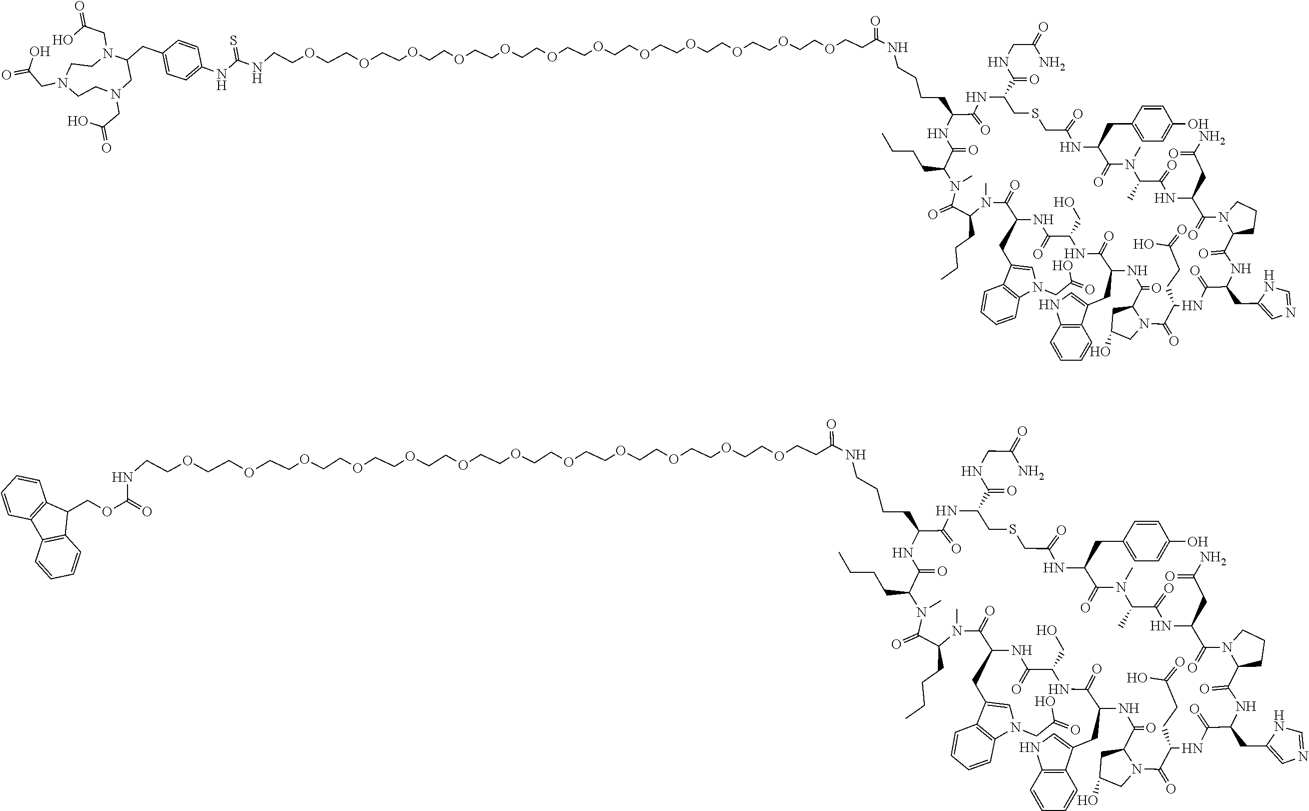

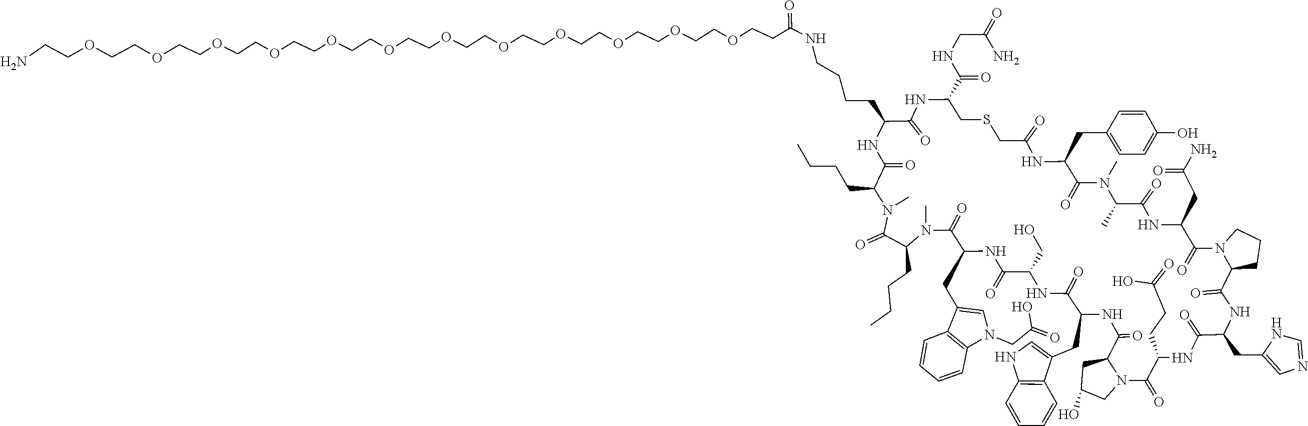 [SMILES: CCCC[C@H]1C(=O)N(C)[C@@H](CCCC)C(=O)N[C@@H](CCCCNC(=O)CCOCCOCCOCCOCCOCCOCCOCCOCCOCCOCCOCCOCCN)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](Cc2cnc[nH]2)C(=O)N[C@@H](CCC(=O)O)C(=O)N2C[C@H](O)C[C@H]2C(=O)N[C@@H](Cc2c[nH]c3ccccc23)C(=O)N[C@@H](CO)C(=O)N[C@@H](Cc2cn(CC(=O)O)c3ccccc23)C(=O)N1C]

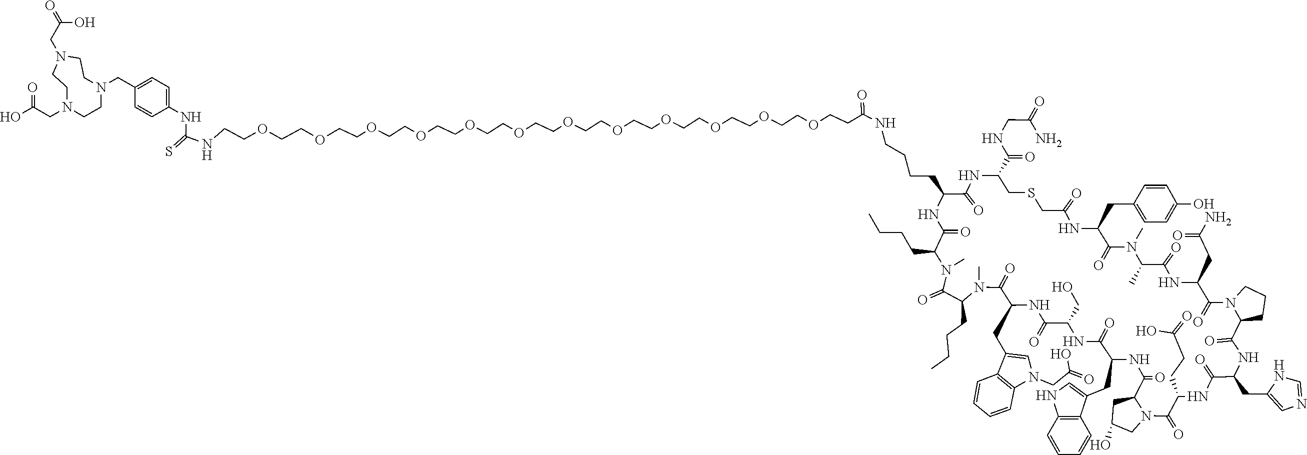 [SMILES: CCCC[C@H]1C(=O)N(C)[C@@H](CCCC)C(=O)N[C@@H](CCCCNC(=O)CCOCCOCCOCCOCCOCCOCCOCCOCCOCCOCCOCCOCCNC(=S)Nc2ccc(CN3CCN(CC(=O)O)CCN(CC(=O)O)CC3)cc2)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](Cc2cnc[nH]2)C(=O)N[C@@H](CCC(=O)O)C(=O)N2C[C@H](O)C[C@H]2C(=O)N[C@@H](Cc2c[nH]c3ccccc23)C(=O)N[C@@H](CO)C(=O)N[C@@H](Cc2cn(CC(=O)O)c3ccccc23)C(=O)N1C]

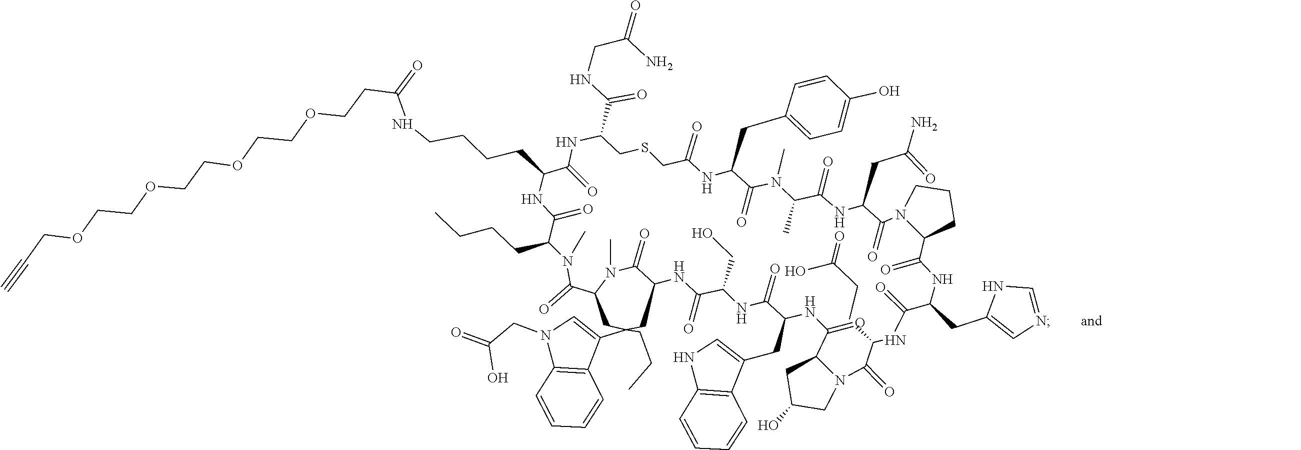 [SMILES: C#CCOCCOCCOCCOCCC(=O)NCCCC[C@@H]1NC(=O)[C@H](CCCC)N(C)C(=O)[C@@H]2CC(CCC)(c3cn(CC(=O)O)c4ccccc34)C[C@H](NC(=O)[C@H](CO)NC(=O)[C@H](Cc3c[nH]c4ccccc34)NC(=O)[C@@H]3C[C@@H](O)CN3C(=O)[C@H](CCC(=O)O)NC(=O)[C@H](Cc3cnc[nH]3)NC(=O)[C@@H]3CCCN3C(=O)[C@H](C(C)C(N)=O)NC(=O)[C@H](C)N(C)C(=O)[C@H](Cc3ccc(O)cc3)NC(=O)CSC[C@@H](C(=O)NCC(N)=O)NC1=O)C(=O)N2C ; "and"]

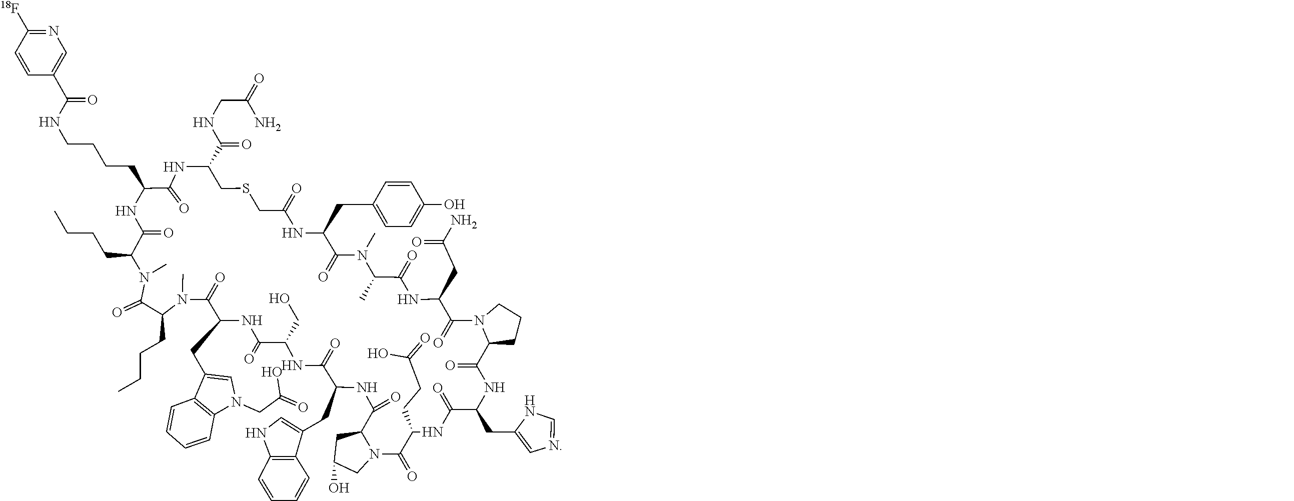 [SMILES: CCCC[C@H]1C(=O)N(C)[C@@H](CCCC)C(=O)N[C@@H](CCCCNC(=O)c2ccc([18F])nc2)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](Cc2cnc[nH]2)C(=O)N[C@@H](CCC(=O)O)C(=O)N2C[C@H](O)C[C@H]2C(=O)N[C@@H](Cc2c[nH]c3ccccc23)C(=O)N[C@@H](CO)C(=O)N[C@@H](Cc2cn(CC(=O)O)c3ccccc23)C(=O)N1C]

In some embodiments, the imaging agent is capable of detecting PD-L1 in vitro, in vivo, and/or ex vivo. In some embodiments, the imaging agent is capable of detecting PD-L1 in vivo. PD-L1 is expressed by a variety of tumors, and its over-expression is induced in tumor cells as an adaptive mechanism in response to tumor infiltrating cytotoxic T-cells. One of ordinary skill will recognize that PD-L1 may comprise modifications and/or mutations and still be applicable for the presently disclosed methods, as long as it still can be detected by a presently disclosed imaging agent.

In some embodiments, the IC₅₀ of a presently disclosed imaging agent to inhibit PD-L1 interaction with its ligand Programmed Cell Death Protein 1 (PD-1) has a range from about 100 nM to about 1 μM. In some embodiments, the IC₅₀ is less than 100 nM, in other embodiments, less than 10 nM, in other embodiments, less than 8 nM, in other embodiments, less than 5 nm, in other embodiments, less than 4 nm, and in other embodiments, less than 3 nM.

The term “binding affinity” is a property that describes how strongly two or more compounds associate with each other in a non-covalent relationship. Binding affinities can be characterized qualitatively, (such as “strong”, “weak”, “high”, or “low”) or quantitatively (such as measuring the K_{d}).

### B. Methods of Detection Using Imaging Agents

In some embodiments, the presently disclosed subject matter provides methods for detecting an immune checkpoint protein, such as PD-L1. In some embodiments, the presently disclosed subject matter provides methods for detecting diseases, disorders, or conditions that result in over-expression of PD-L1, such as cancer, inflammation, infection, and the like.

Accordingly, in some embodiments, the presently disclosed subject matter provides an imaging method for detecting Programmed Death Ligand 1 (PD-L1), the method comprising: (a) providing an effective amount of an imaging agent of formula (I); (b) contacting one or more cells or tissues with the imaging agent; and (c) making an image to detect PD-L1.

As used herein, the term “imaging” or “making an image” refers to the use of any imaging technology to visualize a detectable compound by measuring the energy emitted by the compound. In some embodiments, the term “imaging” refers to the use of any imaging technology to visualize a detectable compound after administration to a subject by measuring the energy emitted by the compound after localization of the compound following administration. In some embodiments, imaging techniques involve administering a compound to a subject that can be detected externally to the subject. In some embodiments, images are generated by virtue of differences in the spatial distribution of the imaging agents that accumulate in various locations in a subject. In some embodiments, administering an imaging agent occurs by injection.

The term “imaging agent” is intended to include a compound that is capable of being imaged by, for example, positron emission tomography (PET). As used herein, “positron emission tomography imaging” or “PET” incorporates a positron emission tomography imaging systems or equivalents and all devices capable of positron emission tomography imaging. The methods of the presently disclosed subject matter can be practiced using any such device, or variation of a PET device or equivalent, or in conjunction with any known PET methodology. See, e.g., U.S. Pat. Nos. 6,151,377; 6,072,177; 5,900,636; 5,608,221; 5,532,489; 5,272,343; 5,103,098, each of which is incorporated herein by reference. Animal imaging modalities are included, e.g., micro-PETs (Corcorde Microsystems, Inc.).

Depending on the reporting moiety, the presently disclosed imaging agents can be used in PET, single-photon emission computed tomography (SPECT), near-infrared (fluorescence), photoacoustic, and Raman imaging.

In some embodiments, the imaging includes scanning the entire subject or patient, or a particular region of the subject or patient using a detection system, and detecting the signal. The detected signal is then converted into an image. The resultant images should be read by an experienced observer, such as, for example, a physician. Generally, imaging is carried out about 1 minute to about 48 hours following administration of the imaging agent. The precise timing of the imaging will be dependent upon such factors as the clearance rate of the compound administered, as will be readily apparent to those skilled in the art. The time frame of imaging may vary based on the radionucleotide being used. In particular embodiments, imaging is carried out between about 1 minute and about 4 hours following administration, such as between 15 minutes and 30 minutes, between 30 minutes and 45 minutes, between 45 minutes and 60 minutes, between 60 minutes and 90 minutes, and between 60 minutes and 120 minutes. In some embodiments, detection of the PD-L1 occurs as soon as about 60 minutes after administration of the imaging agent to the subject. In some embodiments, the imaging may take place 24 hours post injection with a peptide labeled with Zr-89. In some embodiments, the imaging may take place 24 hours post injection with a peptide labeled with I-124.

Once an image has been obtained, one with skill in the art can determine the location of the compound. Using this information, the artisan can determine, for example, if a condition, such as an infection, inflammation, or cancer, is present, the extent of the condition, or the efficacy of the treatment that the subject is undergoing.

In some embodiments, contacting the cells or tissues with the imaging agent is performed in vitro, in vivo, or ex vivo. “Contacting” means any action that results in at least one imaging agent of the presently disclosed subject matter physically contacting at least one cell or tissue. It thus may comprise exposing the cell(s) or tissue(s) to the imaging agent in an amount sufficient to result in contact of at least one imaging agent with at least one cell or tissue. In some embodiments, the method can be practiced in vitro or ex vivo by introducing, and preferably mixing, the imaging agent and cells or tissues in a controlled environment, such as a culture dish or tube. In some embodiments, the method can be practiced in vivo, in which case contacting means exposing at least one cell or tissue in a subject to at least one imaging agent of the presently disclosed subject matter, such as administering the imaging agent to a subject via any suitable route. In some embodiments, contacting the cells or tissues with the imaging agent is performed in a subject.

The term “effective amount” of an imaging agent is the amount necessary or sufficient to provide a readable signal when imaged using the techniques described herein, e.g., positron emission tomography (PET). The effective amount can vary depending on such factors as the size and weight of the subject, the type of illness, or the particular compound. For example, the choice of the compound can affect what constitutes an “effective amount.” One of ordinary skill in the art would be able to study the factors contained herein and make the determination regarding the effective amount of the compound without undue experimentation.

The subject diagnosed or treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the diagnosis or treatment of an existing disease, disorder, condition or an animal subject for medical, veterinary purposes, or developmental purposes. Suitable animal subjects include mammals including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a disease, disorder, or condition. Thus, the terms “subject” and “patient” are used interchangeably herein. Subjects also include animal disease models (e.g., rats or mice used in experiments, and the like). In some embodiments, the subject is a human, rat, mouse, cat, dog, horse, sheep, cow, monkey, avian, or amphibian.

Generally, the presently disclosed imaging agents can be administered to a subject for detection of a disease, disorder, or condition by any suitable route of administration, including orally, nasally, transmucosally, ocularly, rectally, intravaginally, or parenterally, including intravenous, intramuscular, subcutaneous, intramedullary injections, as well as intrathecal, direct intraventricular, intravenous, intra-articular, intra-stemal, intra-synovial, intra-hepatic, intralesional, intracranial, intraperitoneal, intranasal, or intraocular injections, intracisternally, topically, as by powders, ointments or drops (including eyedrops), including buccally and sublingually, transdermally, through an inhalation spray, or other modes of delivery known in the art.

The phrases “systemic administration”, “administered systemically”, “peripheral administration” and “administered peripherally” as used herein mean the administration of compositions such that they enter the subject's or patient's system and, thus, are subject to metabolism and other like processes, for example, subcutaneous or intravenous administration.

The phrases “parenteral administration” and “administered parenterally” as used herein mean modes of administration other than enteral and topical administration, usually by injection, and includes, without limitation, intravenous, intramuscular, intarterial, intrathecal, intracapsular, intraorbital, intraocular, intracardiac, intradermal, intraperitoneal, transtracheal, subcutaneous, subcuticular, intraarticular, subcapsular, subarachnoid, intraspinal and intrasternal injection and infusion.

In some embodiments, the imaging agent exhibits a target to non-target ratio of at least 3:1. In some embodiments, the term “target” refers to the cells or tissues that show over-expression of the PD-L1 protein and the term “non-target” refers to cells or tissues that do not show over-expression of the PD-L1 protein.

In some embodiments, the imaging method is used to detect a cancer. A “cancer” in a subject or patient refers to the presence of cells possessing characteristics typical of cancer-causing cells, for example, uncontrolled proliferation, loss of specialized functions, immortality, significant metastatic potential, significant increase in anti-apoptotic activity, rapid growth and proliferation rate, and certain characteristic morphology and cellular markers. In some circumstances, cancer cells will be in the form of a tumor; such cells may exist locally within an animal, or circulate in the blood stream as independent cells, for example, leukemic cells. Cancer as used herein includes newly diagnosed or recurrent cancers, including without limitation, blastomas, carcinomas, gliomas, leukemias, lymphomas, melanomas, myeloma, and sarcomas. Cancer as used herein includes, but is not limited to, head cancer, neck cancer, head and neck cancer, lung cancer, breast cancer, such as triple negative breast cancer, prostate cancer, colorectal cancer, esophageal cancer, stomach cancer, leukemia/lymphoma, uterine cancer, skin cancer, endocrine cancer, urinary cancer, pancreatic cancer, gastrointestinal cancer, ovarian cancer, cervical cancer, renal cancer, bladder cancer, brain cancer, and adenomas. In some embodiments, the cancer comprises Stage 0 cancer. In some embodiments, the cancer comprises Stage I cancer. In some embodiments, the cancer comprises Stage II cancer. In some embodiments, the cancer comprises Stage III cancer. In some embodiments, the cancer comprises Stage IV cancer. In some embodiments, the cancer is refractory and/or metastatic.

A “tumor,” as used herein, refers to all neoplastic cell growth and proliferation, whether malignant or benign, and all precancerous and cancerous cells and tissues. A “solid tumor”, as used herein, is an abnormal mass of tissue that generally does not contain cysts or liquid areas. A solid tumor may be in the brain, colon, breasts, prostate, liver, kidneys, lungs, esophagus, head and neck, ovaries, cervix, stomach, colon, rectum, bladder, uterus, testes, and pancreas, as non-limiting examples. In some embodiments, the imaging method is used to detect a solid tumor. In yet other embodiments, the imaging method is used to detect a metastatic cancer.

In some embodiments, the imaging method is used to detect an infection. Infectious disease, such as infection by any fungi or bacteria, is contemplated for detection using the presently disclosed subject matter. As used herein, the term “infection” refers to the invasion of a host organism's bodily tissues by disease-causing organisms, their multiplication, and the reaction of host tissues to these organisms and the toxins they produce. Infections include, but are not restricted to, nosocomial infections, surgical infections, and severe abdominal infections, such as peritonitis, pancreatitis, gall bladder empyema, and pleura empyema, and bone infections, such as osteomyelitis. Detection of septicemia, sepsis and septic shock, infections due to or following use of immuno-suppressant drugs, cancer chemotherapy, radiation, contaminated i.v. fluids, haemorrhagic shock, ischaemia, trauma, cancer, immuno-deficiency, virus infections, and diabetes are also contemplated. Examples of microbial infection, such as bacterial and/or fungal infection include, but are not limited to, infections due to Mycobacterium tuberculosis, E. coli, Klebsiella sp., Enterobacter sp., Proteus sp., Serratia marcescens, Pseudomonas aeruginosa, Staphylococcus spp., including S. aureus and coag.-negative Staphylococcus, Enterococcus sp., Streptococcus pneumoniae, Haemophilus influenzae, Bacteroides spp., Acinetobacter spp., Helicobacter spp., Candida sp., etc. Infections due to resistant microbes are included, for example methicillin-resistant Staphylococcus aureus (MRSA) and vancomycin-resistant Enterococcus faecalis (VRE). In some embodiments, the infection is a bacterial infection. In some embodiments, the infection is a chronic bacterial infection. In some embodiments, the bacterial infection is tuberculosis. In some embodiments, the infection is disseminated tuberculosis. In some embodiments, the infection may be hepatitis A, hepatitis B, hepatitis C, and/or human immunodeficiency virus.

In some embodiments, the imaging method is used to detect inflammation. Examples of disorders associated with inflammation include, but are not limited to, asthma, autoimmune diseases, autoinflammatory diseases, Celiac disease, diverticulitis, glomerulonephritis, hidradenitis suppurativa, hypersensitivities, inflammatory bowel diseases, interstitial cystitis, otitis, pelvic inflammatory disease, reperfusion injury, rheumatic fever, rheumatoid arthritis, sarcoidosis, transplant rejection, lupus, including, systemic lupus erythematosus, and vasculitis. In some embodiments, the inflammation is caused by rheumatoid arthritis or systemic lupus erythematosus.

PD-L1 binds to its receptor, PD-1, found on activated T cells, B cells, and myeloid cells, to modulate activation or inhibition. PD-L1 is also expressed on several immune cells including macrophages. Accordingly, the presently disclosed imaging agents, which detect PD-L1 expression, can be used to detect immune cells, such as T cells, B cells, and myeloid cells. In some embodiments, the presently disclosed imaging agents detect immune cells in a tumor. In some embodiments, the presently disclosed imaging agents detect the distribution of immune cells systemically in a subject. In some embodiments, the imaging method is used to detect immune cell responses in infectious cells. In some embodiments, the imaging method is used to detect immune cell responses in inflammatory cells.

In some embodiments, the presently disclosed imaging method detects and/or measures a change in PD-L1 expression, such as a treatment-induced change in PD-L1 expression. Such methods can be used to ascertain the efficacy of a particular treatment method and/or to determine efficacious therapeutic dosage ranges.

### C. Kits Comprising Imaging Agents

In some embodiments, the presently disclosed subject matter provides a kit for detecting Programmed Death Ligand 1 (PD-L1), the kit comprising an imaging agent comprising a compound of formula (I), as described hereinabove.

Typically, the kits of the presently disclosed subject matter comprise a presently disclosed imaging agent and instructions for how to perform at least one presently disclosed method. The imaging agent is generally supplied in the kits in an amount sufficient to detect PD-L1 in at least one subject or patient at least one time. The kits can also comprise some or all of the other reagents and supplies necessary to perform at least one embodiment of the presently disclosed method.

In its simplest form, a kit according to the presently disclosed subject matter comprises a container containing at least one type of imaging agent according to the presently disclosed subject matter. In some embodiments, the kit comprises multiple containers, each of which may contain at least one imaging agent or other substances that are useful for performing one or more embodiments of the presently disclosed methods.

The container can be any material suitable for containing a presently disclosed composition or another substance useful in performing a presently disclosed method. Thus, the container may be a vial or ampule. It can be fabricated from any suitable material, such as glass, plastic, metal, or paper or a paper product. In embodiments, it is a glass or plastic ampule or vial that can be sealed, such as by a stopper, a stopper and crimp seal, or a plastic or metal cap. The amount of imaging agent contained in the container can be selected by one of skill in the art without undue experimentation based on numerous parameters that are relevant according to the presently disclosed subject matter.

In embodiments, the container is provided as a component of a larger unit that typically comprises packaging materials (referred to below as a kit for simplicity purposes). The presently disclosed kit can include suitable packaging and instructions and/or other information relating to the use of the compositions. Typically, the kit is fabricated from a sturdy material, such as cardboard and plastic, and can contain the instructions or other information printed directly on it. The kit can comprise multiple containers containing the composition of the invention. In such kits, each container can be the same size, and contain the same amount of composition, as each other container, or different containers may be different sizes and/or contain different amounts of compositions or compositions having different constituents. One of skill in the art will immediately appreciate that numerous different configurations of container sizes and contents are envisioned by this invention, and thus not all permutations need be specifically recited herein.

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### 1.1 Results

1.1.1 Synthesis and in vitro evaluation of a hydrophilic PD-L1-specific PET imaging agent. PD-L1 detection using IHC is a guiding tool for PD-1:PD-L1 therapy. McLaughlin et al., 2016. Tools to quantify total PD-L1 levels in all of the lesions non-invasively, however, have emerged only recently and are in early clinical evaluation. Bensch et al., 2018; Niemeijer et al., 2018. Quantifying PD-L1 dynamics presents a different challenge, however, due to the need for PET imaging agents that provide high contrast images within the standard clinical workflow.

To address this need, a PD-L1-specific peptide-based imaging agent, [⁶⁴Cu]WL12, was developed previously and its potential to detect tumor PD-L1 levels was demonstrated. Kumar et al., 2019. [⁶⁴Cu]WL12, however, is lipophilic and shows high non-specific accumulation in several tissues including liver. Kumar et al., 2019; Chatterjee et al., 2017. To improve the imaging properties, a new hydrophilic peptide was identified and a radiofluorinated analog was generated using the aluminum fluoride method to facilitate clinical translation.

DK221 is a 14 amino acid human PD-L1-specific cyclic peptide with three carboxylate groups and a free lysine amine. Miller et al., 2016. The structure of DK221 is shown immediately herein below, with the free lysine amine annotated with an *:

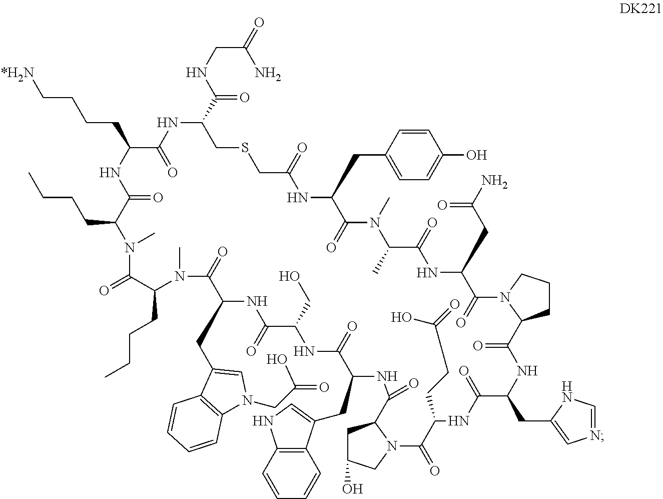

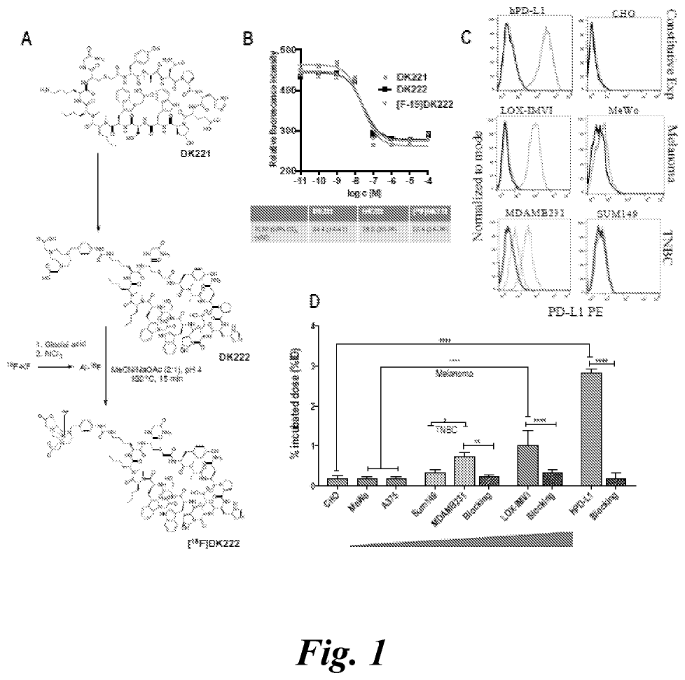

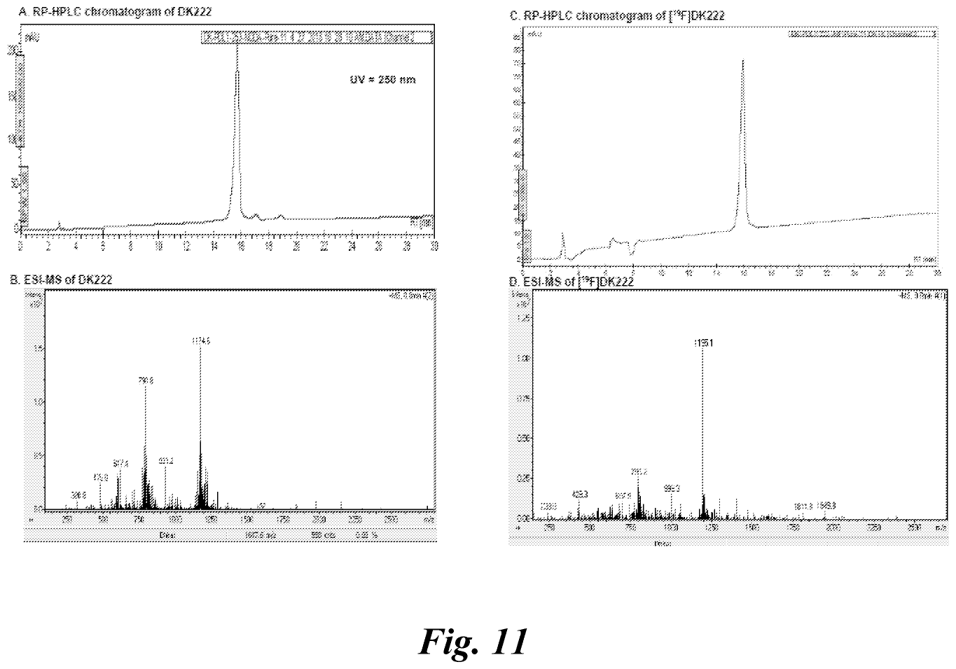

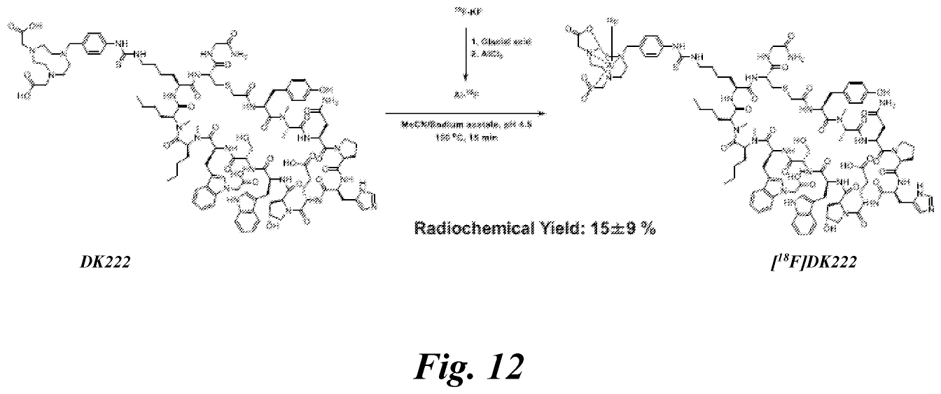

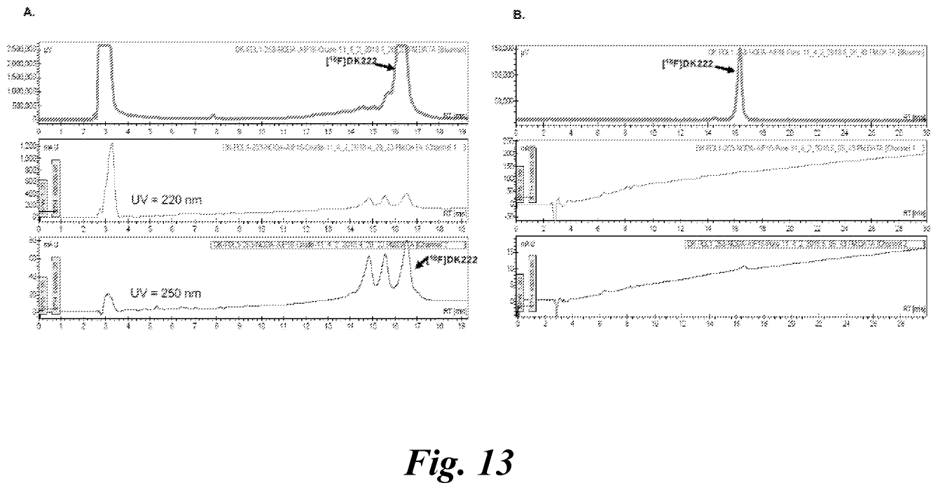

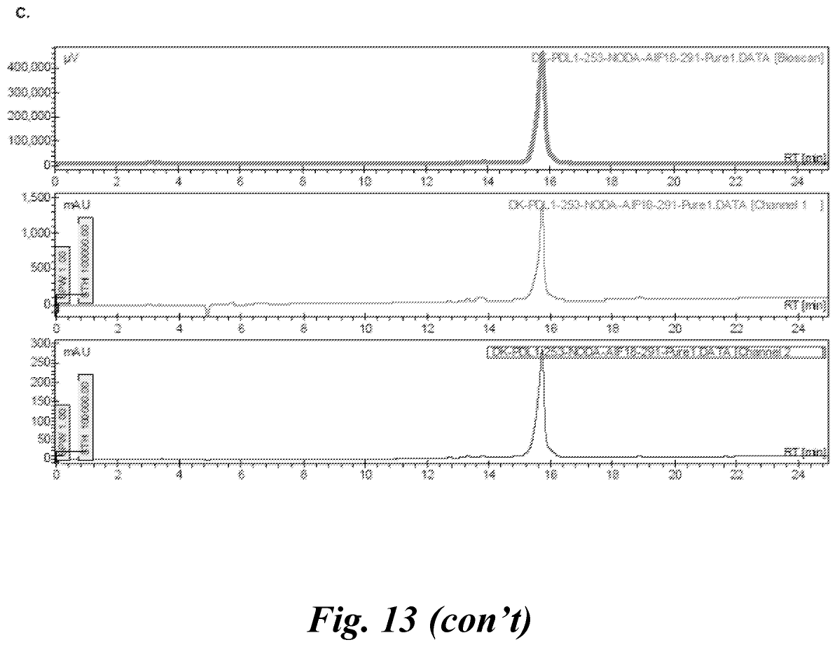

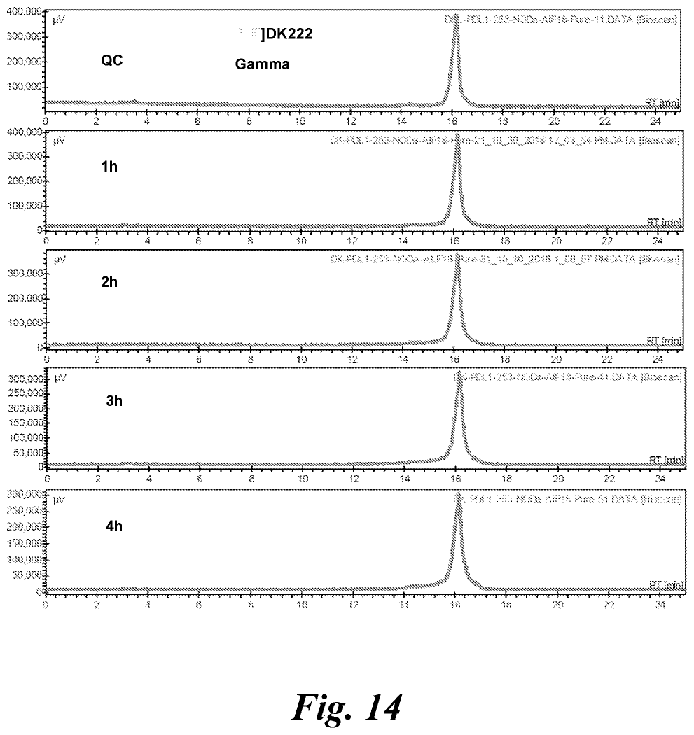

To modify DK221 for radiolabeling, a bifunctional chelator, e.g., NCS-MP-NODA, was conjugated to the free lysine amine to generate DK222. The NODA chelator was used for radiofluorination to produce [¹⁸F]DK222, as well as a non-radioactive analog [¹⁹F]DK222. (FIG. 1A, FIG. 10 and FIG. 11). A competitive PD-1:PD-L1 inhibition assay was performed to characterize binding affinity of the peptide analogs to PD-L1. Peptide analogs were observed to dose-dependently inhibit PD-L1 binding to PD-1 with IC₅₀ values of 24, 28, and 25 nM for DK221, DK222, and [¹⁹F]DK222, respectively (FIG. 1B). [¹⁸F]Fluoride-radiolabeling of peptides and small molecules by aluminum fluoride (AlF) method is gaining attention due to the ease of synthesis and the potential to retain the hydrophilicity of the binding moiety. McBride et al., 2010; Kumar et al., 2018. The radiolabeled analog, [¹⁸F]DK222 was synthesized by AlF method in good radiochemical yields (34.85±1.7%, n=62), in vitro stability, and moderate specific activity of 284±56 mCi/μmol (10.51±2.07 GBq/μmol, n=25). Formulated [¹⁸F]DK222 was found to be stable for 4 hours at the prepared radioactivity concentrations. (FIG. 12, FIG. 13 and FIG. 14).

Cell binding assays were performed to assess the specificity of [¹⁸F]DK222 to PD-L1. CHO cells with constitutive human PD-L1 expression (hPD-L1) and multiple cancer cell lines of triple negative breast cancer (TNBC) (MDAMB231, SUM149) and melanoma (LOX-IMVI, MeWo, and A375) origin were selected. Cells were incubated with [¹⁸F]DK222 at 4° C. for 30 min, washed thoroughly, and cell-bound activity was measured. Uptake of [¹⁸F]DK222 reflected the variable levels of surface PD-L1 expression observed by flow cytometry (FIG. 1C and FIG. 1D) in the order: hPD-L1>LOX-IMVI>MDAMB231>Sum149. A375, CHO, and MeWo cells, which expressed low PD-L1 levels, exhibited the least [¹⁸F]DK222 binding.

Binding studies in the presence of 1 μM excess of the parent DK221 peptide were performed to validate the specificity of [¹⁸F]DK222 for PD-L1. A greater than 90% reduction in radioactivity uptake in PD-L1-positive cells (P<0.0001) was observed. Taken together, these in vitro results provided evidence that [¹⁸F]DK222 binding is specific to PD-L1.

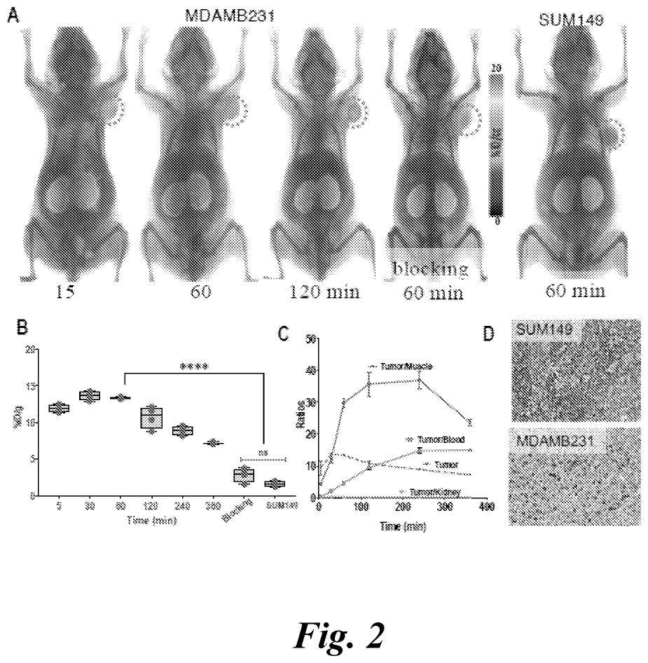

1.1.2 Evaluation of [⁸F]DK222 biodistribution in mouse models of TNBC. To gain insight into the PK and biodistribution of [¹⁸F]DK222, PET imaging studies were performed in immunocompromised NSG mice harboring PD-L1-positive MDAMB231 xenografts. PET images acquired at 15, 60, and 120 min after [¹⁸F]DK222 injection showed high radiotracer accumulation in tumors as early as 15 mn (FIG. 2A). In addition to tumors, kidneys showed the highest uptake of radioactivity at all the time points investigated. That high and selective uptake of [¹⁸F]DK222 in tumors combined with fast clearance from normal tissues provided high contrast images at 60-120 min after [¹⁸F]DK222 injection. Reduced uptake of radiotracer was observed in SUM149 tumors, which expresses low PD-L1 levels, and in mice that received a blocking dose (50 mg/kg) of the parent DK221 peptide.

Ex vivo measurements at 5, 30, 60, 120, 240, and 360 min after radiotracer injection were conducted to validate imaging studies and to quantify [¹⁸F]DK222 biodistribution in normal tissues (Table 1).

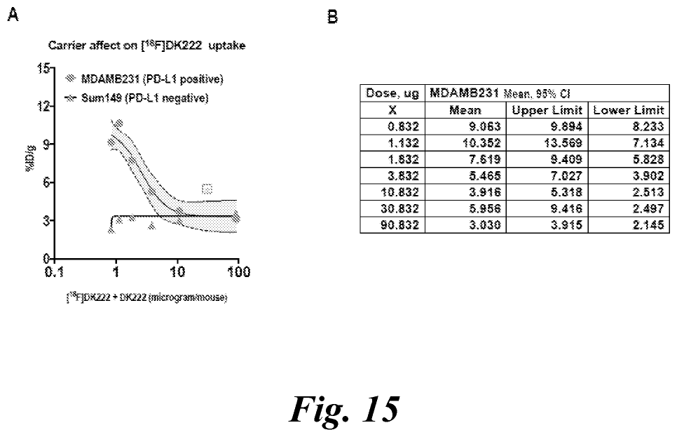

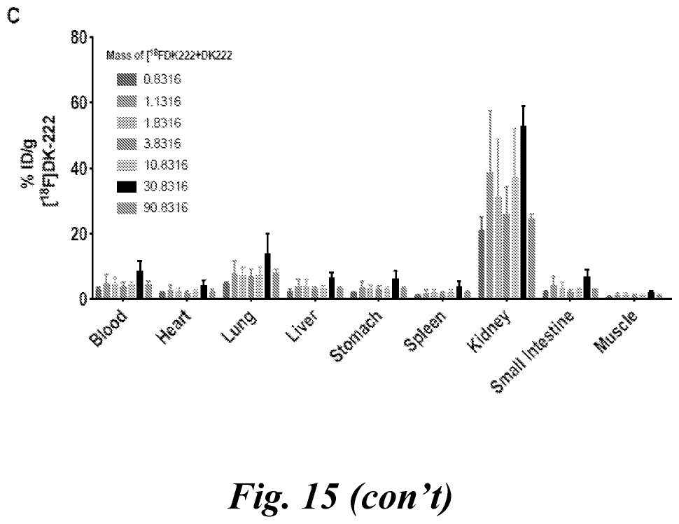

TABLE 1
[¹⁸F]DK222 ex vivo biodistribution studies in NSG mice bearing MDAMB231 xenografts.

| | MDAMB231 | | | | | | | |
|---|---|---|---|---|---|---|---|---|
| | | | | | | | 60 min | SUM149 |
| | 5 min | 30 min | 60 min | 120 min | 240 min | 360 min | blocking | 60 min |
| Blood | 24.7 ± 1.2 | 6.8 ± 0.1 | 3.0 ± 0.1 | 1.1 ± 0.1 | 0.6 ± 0.1 | 0.5 ± 0.0 | 4.5 ± 0.7 | 2.6 ± 0.7 |
| Thymus | 19.2 ± 3.2 | 8.1 ± 0.7 | 3.7 ± 0.5 | 2.6 ± 0.2 | 2.0 ± 0.2 | 2.0 ± 0.1 | 6.8 ± 0.9 | 4.4 ± 0.5 |
| Heart | 8.5 ± 0.4 | 4.2 ± 1.6 | 1.3 ± 0.0 | 0.7 ± 0.0 | 0.5 ± 0.0 | 0.6 ± 0.0 | 2.0 ± 0.3 | 1.0 ± 0.1 |
| Lung | 22.0 ± 1.0 | 11.0 ± 1.0 | 4.2 ± 0.5 | 1.7 ± 0.2 | 0.8 ± 0.0 | 0.7 ± 0.0 | 7.0 ± 1.4 | 3.4 ± 0.3 |
| Liver | 13.7 ± 0.5 | 5.1 ± 1.1 | 44 ± 0.0 | 3.6 ± 0.2 | 2.8 ± 0.2 | 2.8 ± 0.2 | 5.2 ± 0.7 | 2.7 ± 0.6 |
| Stomach | 2.3 ± 0.2 | 1.2 ± 0.2 | 1.0 ± 0.3 | 0.3 ± 0.0 | 0.6 ± 0.3 | 0.3 ± 0.0 | 1.0 ± 0.2 | 0.8 ± 0.2 |
| Pancreas | 4.3 ± 0.4 | 1.6 ± 0.1 | 0.8 ± 0.0 | 0.5 ± 0.0 | 0.4 ± 0.0 | 0.5 ± 0.1 | 1.3 ± 0.2 | 0.5 ± 0.0 |
| Spleen | 10.3 ± 2.5 | 3.8 ± 0.3 | 1.8 ± 0.1 | 1.5 ± 0.1 | 1.1 ± 0.0 | 1.6 ± 0.1 | 3.2 ± 0.5 | 1.7 ± 0.2 |
| Adrenals | 8.1 ± 1.1 | 3.3 ± 0.3 | 2.0 ± 0.1 | 1.9 ± 0.3 | 2.0 ± 0.5 | 2.0 ± 0.2 | 4.9 ± 2.4 | 1.0 ± 0.2 |
| Kidney | 57.0 ± 4.0 | 53.9 ± 1.7 | 57.7 ± 0.5 | 61.9 ± 4.3 | 51.9 ± 3.0 | 43.3 ± 2.2 | 45.6 ± 7.5 | 37.0 ± 3.5 |
| Small Intestines | 4.4 ± 1.2 | 2.2 ± 0.1 | 1.3 ± 0.0 | 0.9 ± 0.1 | 0.6 ± 0.1 | 0.8 ± 0.1 | 2.0 ± 0.3 | 0.9 ± 0.1 |
| Large Intestines | 5.3 ± 1.1 | 1.8 ± 0.4 | 1.7 ± 0.4 | 1.2 ± 0.3 | 0.7 ± 0.1 | 2.4 ± 1.3 | 1.9 ± 0.4 | 0.8 ± 0.2 |
| Ovaries | 6.2 ± 0.3 | 2.5 ± 0.6 | 1.1 ± 0.3 | 1.0 ± 0.2 | 0.7 ± 0.2 | 1.8 ± 0.3 | 2.0 ± 0.8 | 1.6 ± 0.4 |
| Uterus | 8.0 ± 0.8 | 3.7 ± 0.3 | 1.8 ± 0.1 | 1.2 ± 0.2 | 0.7 ± 0.0 | 0.8 ± 0.1 | 3.3 ± 0.6 | 1.3 ± 0.2 |
| Muscle | 2.8 ± 0.1 | 1.1 ± 0.1 | 0.5 ± 0.0 | 0.3 ± 0.0 | 0.2 ± 0.0 | 0.3 ± 0.0 | 1.0 ± 0.2 | 0.5 ± 0.0 |
| Tumor | 11.7\| ± 0.2 | 13.7 ± 0.3 | 18.4 ± 0.1 | 10.7 ± 0.8 | 8.9 ± 0.3 | 7.2 ± 0.1 | 2.8 ± 0.5 | 1.6 ± 0.2 |
| Femur | 6.9 ± 1.8 | 2.3 ± 0.2 | 1.1 ± 0.1 | 0.7 ± 0.1 | 0.7 ± 0.1 | 0.7 ± 0.1 | 2.0 ± 0.2 | 1.1 ± 0.3 |
| Brain | 0.7 ± 0.2 | 0.2 ± 0.0 | 0.1 ± 0.0 | 0.1 ± 0.0 | 0.1 ± 0.0 | 0.1 ± 0.0 | 0.1 ± 0.0 | 0.1 ± 0.0 |
| Bladder | 8.3 ± 0.6 | 4.8 ± 0.5 | 2.3 ± 0.2 | 1.8 ± 0.2 | 1.9 ± 0.2 | 4.3 ± 1.1 | 4.5 ± 0.8 | 2.3 ± 0.3 |

[¹⁸F]DK222 uptake consistently remained high in tumors until 4 h after injection (FIG. 2B). Time activity curves (FIG. 2C) plotted from the biodistribution data (expressed as percentage of injected dose per gram of tissue [% ID/g]) showed high accumulation and retention of [¹⁸F]DK222 in MDAMB231 tumors. A steady increase in [¹⁸F]DK222 uptake was observed in tumors until 120 min, followed by slow washout between 120 and 360 min. Consistent with PET imaging, uptake of [¹⁸F]DK222 was consistently higher in tumors and kidneys. Small peptides often demonstrate renal clearance and the high kidney uptake observed indicates renal clearance of [¹⁸F]DK222. A steady decrease in radioactivity was observed with time in blood, muscle, and all other tissues that contributed to high image contrast. The tumor-to-blood and tumor-to-muscle ratios at 60 min were 4.5±0.2 and 30.0±1.3, respectively. High [¹⁸F]DK222 uptake observed in several tissues at early time points, including thymus, lung, liver, and bone, was cleared rapidly within 60 min. [¹⁸F]DK222 uptake in SUM149 tumors was 88% (P<0.0001) less than that seen with MDAMB231 tumors at 60 min. Also, mice receiving the blocking dose showed a 79% reduction (P<0.0001) in [¹⁸F]DK222 uptake. Furthermore, administration of a range of non-radioactive doses reduced uptake in MDAMB231 tumors in a dose-dependent manner but not in SUM149 tumors or other tissues (FIG. 15A-FIG. 15C). These observations were confirmed by strong and weak immunoreactivity for PD-L1 observed in MDAMB231 and SUM149 tumors, respectively (FIG. 2D). Based on the high tumor uptake and high tumor-to-blood (and muscle) ratios observed, all imaging and biodistribution studies in other tumor models were conducted at 60 min as provided herein below.

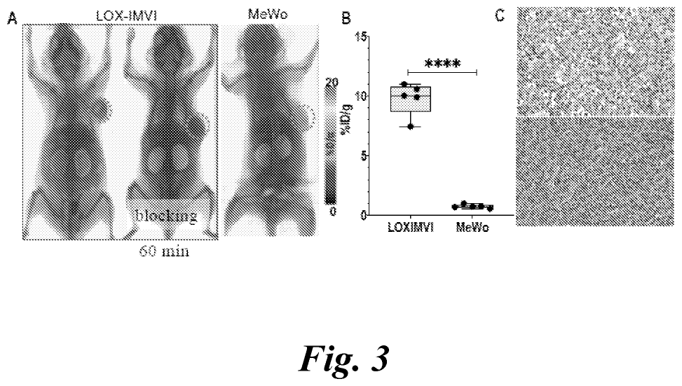

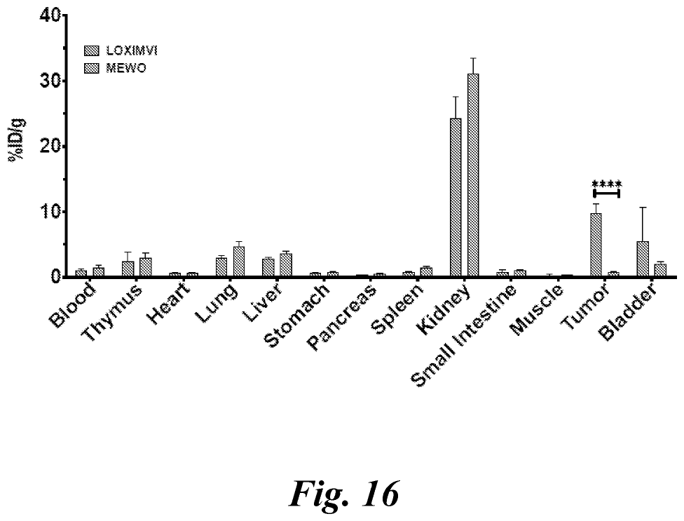

1.1.3. In vivo validation of [⁸F]DK222 specificity in melanoma xenograft models. Next, the PD-L1 specificity of [¹⁸F]DK222 in melanoma models was validated. NSG mice bearing high PD-L1 expressing LOX-IMVI or low PD-L1 expressing MeWo melanoma xenografts and injected with [¹⁸F]DK222 showed high accumulation of radioactivity in LOX-IMVI tumors. [¹⁸F]DK222 uptake was low in MeWo tumors and in mice that received a blocking dose of non-radioactive peptide (FIG. 3A and FIG. 3B, FIG. 16). Supporting the PET imaging results, ex vivo measurement studies conducted at 60 min showed [¹⁸F]DK222 uptake in MeWo tumors to be 95% less compared to LOX-IMVI tumors (P<0.0001). Histological analysis supported the PET imaging findings, in which an intense PD-L1 immunoreactivity in LOX-IMVI tumors (Top panel) but not in MeWo tumors (bottom panel) was observed (FIG. 3C).

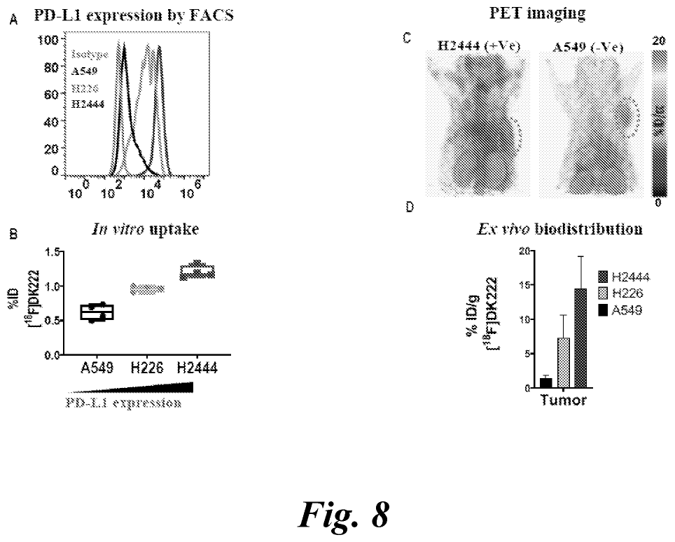

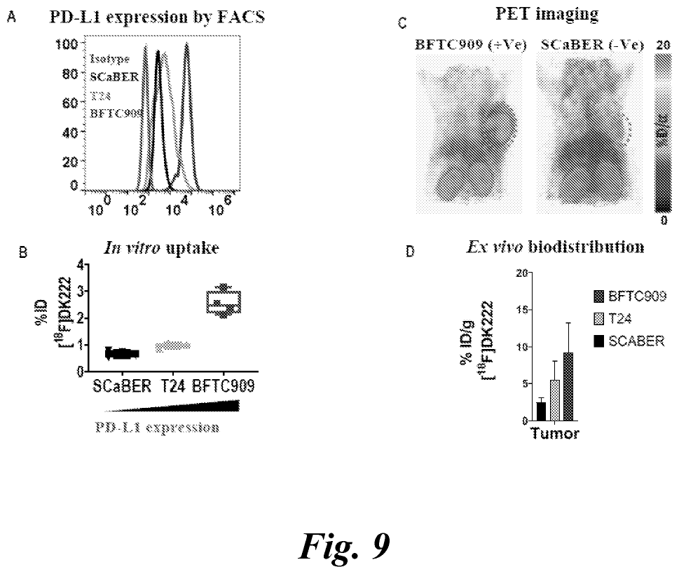

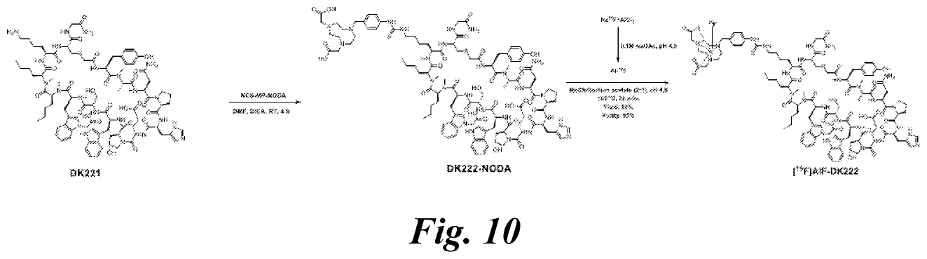

Similar results also were observed in lung and bladder cancer models (FIG. 8 and FIG. 9). Overall, in vivo imaging and ex vivo measurements in TNBC and melanoma models provided further evidence for the specificity of [¹⁸F]DK222 for PD-L1 and its potential to quantify variable PD-L1 levels across different tumor types.

1.1.4. Quantifying pharmacodynamic effects of aPD-1 mAbs at the tumor with [¹⁸F]DK222. The PD-1/PD-L1 pathway represents a cornerstone for combination immune checkpoint blockade regimens. Topalian et al., 2015. Many of those combination therapies converge on the production of IFNγ that is inextricably linked to PD-L1 levels. Minn and Wherry, 2016. Activation of PD-L1 is indicative of robust cytolytic activity that is suppressed by the TME or unleashed by the therapeutics targeting PD-1. Topalian et al., 2015; Minn and Wherry, 2016. Reinvigoration of exhausted T cells can be detected in blood as early as three weeks in patients receiving PD-1 therapeutics. In contrast, their activity at the tumor remains poorly understood. Huang et al., 2019. Immune reinvigoration at the tumor often involves cytolytic activity of immune cells, IFNγ secretion, and induction of PD-L1 levels in the tumor bed. Taube et al., 2012. Thus, without wishing to be bound to any one particular theory, it was thought that tumor PD-L1 levels would be a proximal biomarker to measure the pharmacodynamic effects of PD-1 therapeutics. Therefore, the changes in tumor PD-L1 levels induced by different PD-1 therapeutics were sought to be quantified using PD-L1 PET and to confirm those measurements with immunological responses.

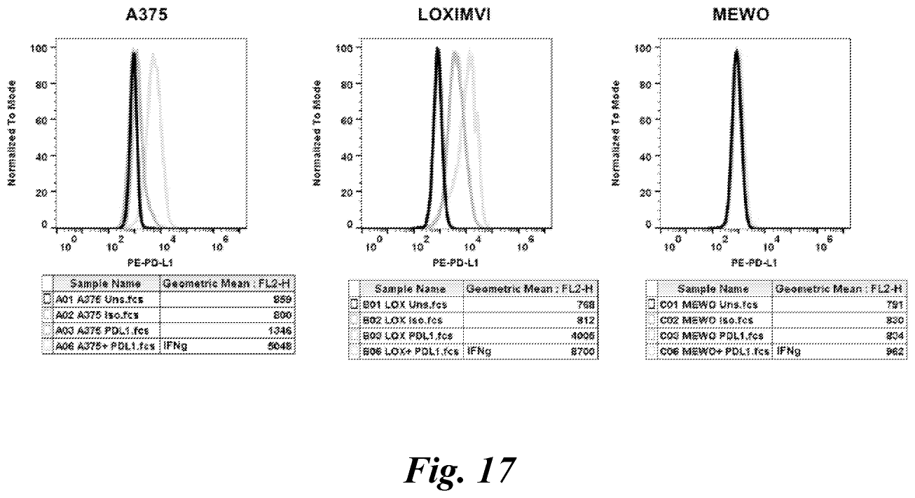

First, the effect of adaptive immune response on the PD-L1 expression in melanoma cells was investigated by assessing changes in PD-L1 levels induced by IFNγ treatment. LOX-IMVI, A375, and MeWO melanoma cells treated with IFNγ were analyzed for changes in PD-L1 levels. Flow cytometry analysis showed a two- and four-fold increase in PD-L1 levels in response to IFNγ treatment in LOX-IMVI and A375 cells, respectively (FIG. 17). No differences were observed in MeWo cells.

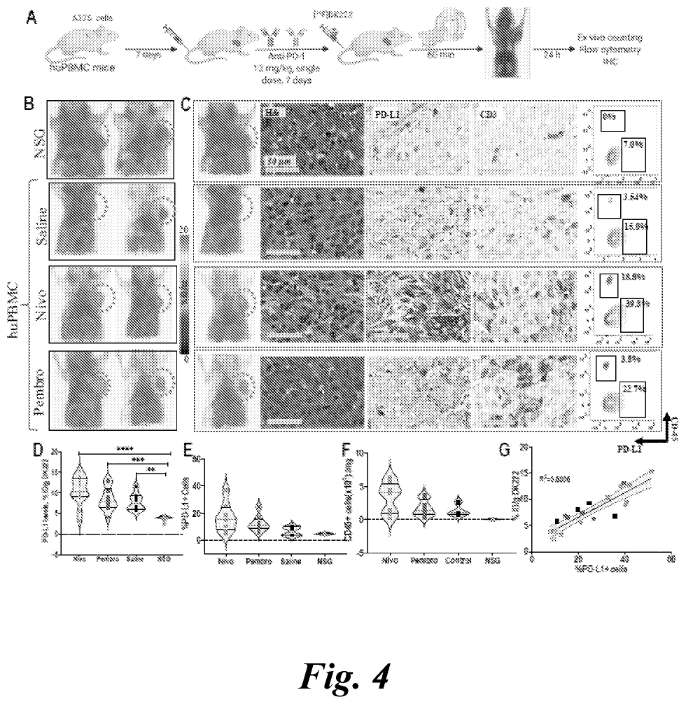

A humanized mouse model was used to quantify the differences in tumor PD-L1 levels as a measure of adaptive immune response to treatment with different aPD-1 mAbs. NSG mice humanized with PBMCs (huPBMC) bearing A375 melanoma xenografts were treated with a single dose of aPD-1 mAbs (12 mg/kg). Following one week of treatment, tumor PD-L1 levels were measured by [¹⁸F]DK222-PET and by ex vivo counting 24 hours later (FIG. 4A). As controls, tumor-bearing huPBMC mice treated with saline and NSG mice treated with Pembrolizumab and Nivolumab were included.

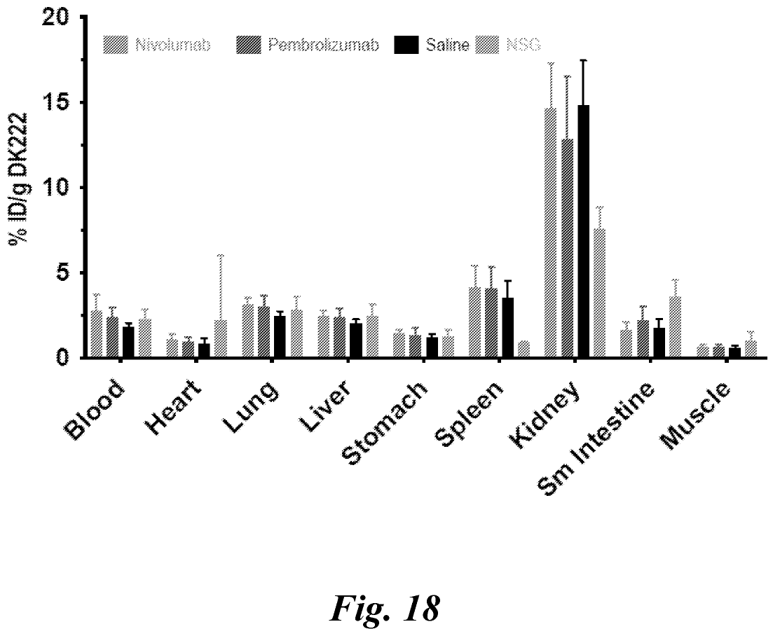

First, whether there are any differences in PD-L1 levels in tumors between humanized and non-humanized mice was assessed. A375 tumors in all the huPBMC mice showed elevated [¹⁸F]DK222 uptake indicating immune cell activity. In contrast, [¹⁸F]DK222 uptake was low in NSG mice lacking huPBMC (% ID/g 8.5 vs. 3.9; P=0.0002). Analysis of tumor sections showed increased immunoreactivity for PD-L1 and CD3 in huPBMC mice vs NSG, validating the PET study results. Increased [¹⁸F]DK222 uptake in the kidneys and spleen of huPBMC mice also was observed compared to those of NSG mice (FIG. 18). In contrast, no significant differences in [¹⁸F]DK222 uptake were observed in nonspecific tissues such as muscle. These results indicated the potential of [¹⁸F]DK222 to differentiate tumors with low PD-L1 levels, and perhaps also those with immune cell exclusion.

Next, the pharmacodynamic effects of different aPD-1 therapeutics at the tumor was assessed. First, the differences in tumor PD-L1 levels between treatment groups in huPBMC mice was examined. Notably, three out of six huPBMC mice treated with vehicle showed high [¹⁸F]DK222 uptake. In contrast, a significant number of mice treated with aPD-1 mAbs showed high [¹⁸F]DK222 uptake with some variability in the tumors (FIG. 4A and FIG. 4B). Validating PET imaging data and revealing differences in therapy induced PD-L1 levels in the TME, biodistribution studies showed a 148, 85, and 76% increase in median [¹⁸F]DK222 uptake in mice treated with Nivolumab, Pembrolizumab, or saline, respectively, compared to NSG mice (FIG. 4C). Analysis of tumor sections from aPD-1 mAb-treated mice showed increased immunoreactivity for PD-L1 and CD3 that is reflective of observed [¹⁸F]DK222. These results indicate that different PD-1 therapeutics exert differing PD effects at the tumor which can be measured as changes in tumor PD-L1 levels.

Tumors were extracted from the mice and flow cytometry analysis was performed to quantify PD-L1 levels to validate that the observed [¹⁸F]DK222 uptake is indeed PD-L1-specific. Increased [¹⁸F]DK222 uptake and total PD-L1 levels were observed in PD-1 treatment groups, which is supported by increased accumulation of CD45+CD8+ immune cells in tumors (FIG. 4D, FIG. 4E and FIG. 4F). A strong correlation between [¹⁸F]DK222 uptake and total PD-L1 levels in the tumors (R²=0.80; P<0.0001, FIG. 4G) and tumor cell-specific PD-L1 levels (R²=0.71; P<0.0001) was observed. In contrast, correlation between [¹⁸F]DK222 uptake and immune cell-specific PD-L1 levels was low, perhaps due to the small contribution from immune cell PD-L1 levels to the total PD-L1 levels in the TME (R²=0.57; P<0.0001) in this model. There also is an increase in CD45+CD8+ cell accumulation in the tumors in treatment groups. [¹⁸F]DK222 uptake in spleen did not correlate with PD-L1 levels. These data establish that [¹⁸F]DK222 PET can be used to quantify PD-L1 dynamics induced by aPD-1 treatments.

Next, to test the hypothesis that [¹⁸F]DK222 uptake can be used to quantify the differential effects of aPD-1 mAbs in the tumor bed, a fixed effects model for statistical analysis was used to quantify heterogeneity in induced PD-L1 levels in the tumor bed. In the fixed effects model, both the fixed aPD-1 mAbs are thought of as specific choice to be compared against each other. Which one of Nivolumab and Pembrolizumab induce PD-L1 more effectively over time at a given dose was investigated. Each aPD-1 mAb is compared against saline, and the difference is tabulated. It was observed, when given at the same dose, subtle, but not statistically significant differences in induced PD-L1 levels between Nivolumab and Pembrolizumab with Nivolumab treatment resulting in greater PD-L1 expression in this model system. Taken together, these data demonstrate that [¹⁸F]DK222 measured in the tumor bed can be used to compare the pharmacodynamic effects of different aPD-1 mAbs early during treatment.

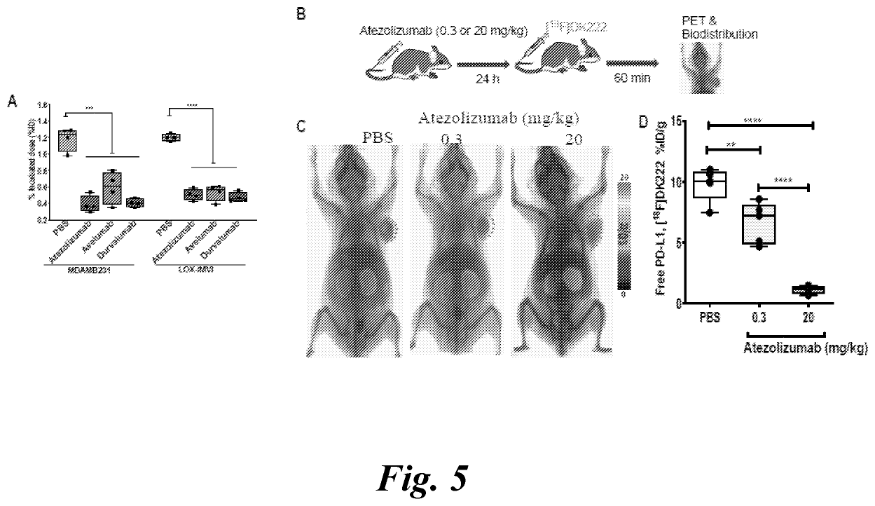

1.1.5. Quantifying accessible tumor PD-L1 levels during aPD-L1 treatment. To quantify accessible PD-L1 levels using [¹⁸F]DK222, first, the interaction of the peptide analogs and aPD-L1 mAbs with PD-L1 protein were studied using bio-layer interferometry. It was found that PD-L1:peptide dissociation constant is at least 100-fold weaker than that of aPD-L1 mAbs. This observation suggests that, at the tracer concentrations used (low nM), [¹⁸F]DK222 will not interfere with anti-PD-L1 therapy. To reproduce these observations in a cell-based system, MDAMB231 and LOX-IMVI cells were incubated with [¹⁸F]DK222 in the presence or absence of 60 nM PD-L1 mAb at 4° C. for 30 min and the bound radioactivity was measured. A greater than 65% reduction in [¹⁸F]DK222 uptake was observed in the presence of mAb in both cell types (P<0.0001), indicating that cell membrane PD-L1 levels are occupied by the mAbs (FIG. 5A). These data indicate that [¹⁸F]DK222 has the potential to quantify accessible PD-L1 levels in vivo and can enable the quantification of accessible PD-L1 levels during treatment.

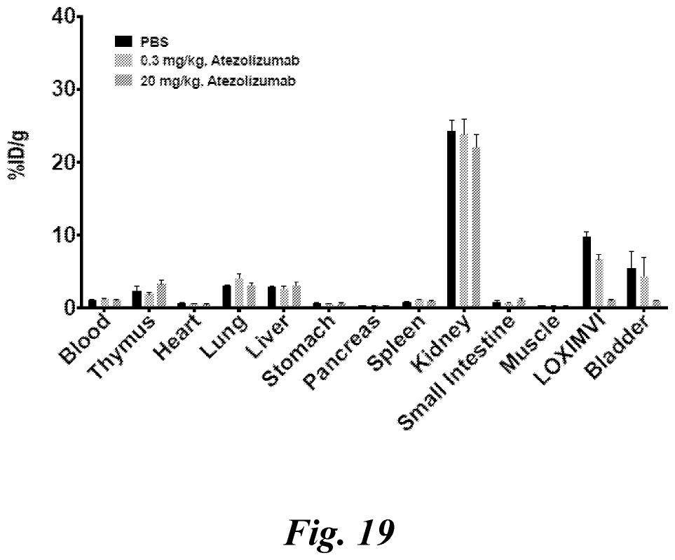

To confirm the in vitro observations in vivo, NSG mice bearing LOX-IMVI tumors were treated with a single dose of 0.3 or 20 mg/kg of Atezolizumab, administered intravenously as a bolus, 24 hour before [¹⁸F]DK222 injection (FIG. 5B, FIG. 5C and FIG. 5D). PET images acquired 60 min after [¹⁸F]DK222 injection showed a significant accumulation of radioactivity in tumors in vehicle-treated controls. In contrast, signal intensity in tumors was significantly reduced in mice receiving 20 mg/kg of mAb (FIG. 5C and FIG. 5D). Importantly, there was a modest reduction in signal intensity when a low 0.3 mg/kg dose of Atezolizumab was used. Ex vivo studies showed a 89% (P<0.0001) and 32% (P<0.01) reduction in [¹⁸F]DK222 uptake in tumors in mice treated with 20 and 0.3 mg/kg of Atezolizumab, respectively, compared to vehicle-treated mice, indicating different accessible PD-L1 levels in the tumors (FIG. 5D, FIG. 19). Taken together, these in vitro and in vivo results demonstrate the potential of [¹⁸F]DK222 PET to measure accessible tumor PD-L1 levels and to identify lesions that are not saturated by the drug treatment.

1.1.6. Accessible Tumor PD-L1 Levels Provide Insights into PK and PD Effects of aPD-L1 mAbs in the Tumor Bed.

The effectiveness of different mAbs targeting PD-L1 in the TME may be heterogeneous because of differing PK and PD, which remain uncharacterized. [¹⁸F]DK222 can bind accessible PD-L1, thus [¹⁸F]DK222 PET signal can show the extent to which PD-L1 remains inaccessible: the lower the signal, the better the PD-L1 mAb targeting efficiency. Insights gained into PK and PD of mAbs during a trial round of immunotherapy could further guide the choice of specific mAb for treatment.

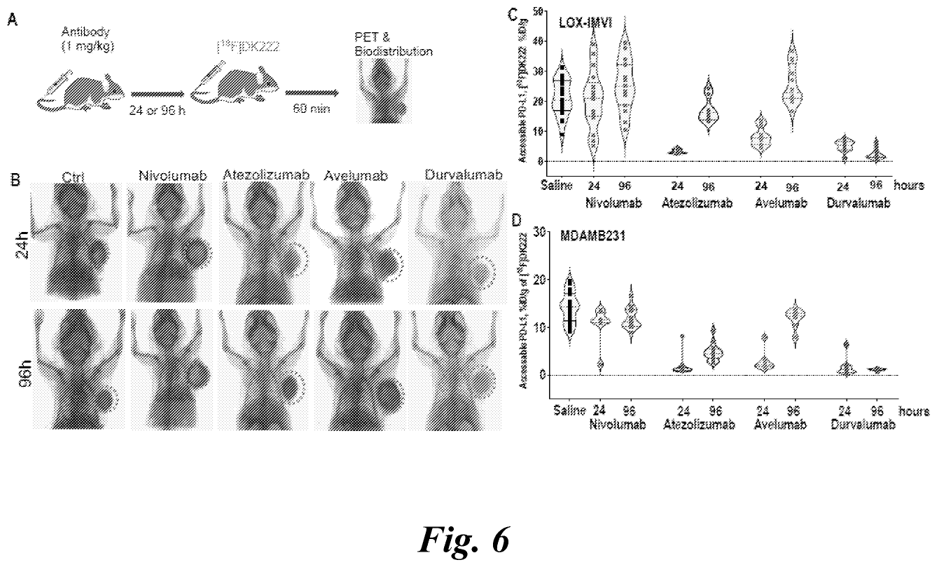

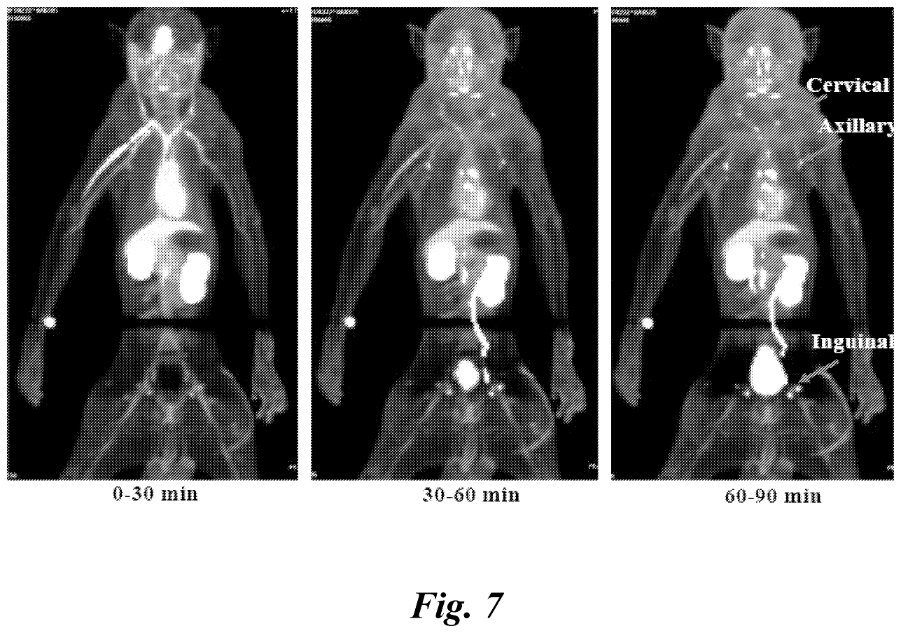

The aim of this experiment was to evaluate the potential of [¹⁸F]DK222 to detect the heterogeneity in binding of different mAbs, thus proving in principle that it can be used to guide the choice between the multiple mAbs available for treatment. For this experiment, three mAbs were chosen (Atezolizumab, Avelumab, Durvalumab), Yu et al., 2019, and Nivolumab was used as an a priori negative control. Separate groups of animals were injected with a single 1 mg/kg dose of (only) one of these mAbs, and after either 24 or 96 hours, each group was injected with [¹⁸F]DK222, imaged, or sacrificed and the [¹⁸F]DK222 signal was quantified (FIG. 6A). The difference in lower [¹⁸F]DK222 signal, at 24 hours between any PD-L1 mAb vs Nivolumab constitutes the saturation binding of the PD-L1 mAb. On the other hand, the higher [¹⁸F]DK222 signal at 96 hours versus that at 24 hours represents loss of the mAb from its binding site, making more PD-L1 accessible to [¹⁸F]DK222. The experiment was repeated in two different tumor models: LOXIMVI and MDAMB231.

PET images of LOX-IMVI tumor-bearing mice showed a significant reduction in [¹⁸F]DK222 in all the groups treated with aPD-L1 mAbs for 24 hours. In contrast, [¹⁸F]DK222 uptake in Nivolumab-treated animals were similar to that of vehicle treatment. A significant increase in [¹⁸F]DK222 uptake was observed at 96 hours in tumors of mice treated with Atezolizumab and Avelumab, but not in mice treated with Durvalumab (FIG. 6B). [¹⁸F]DK222 uptake in Nivolumab-treated mice at 24 and 96 hours was not significant, suggesting that uptake was specific to aPD-L1 mAb treatment. Further analyses were performed to validate these observations.

Two different statistical analysis strategies to quantify heterogeneity of therapeutic mAb binding were used to test the hypothesis that [¹⁸F]DK222 uptake can be used to quantify the differential effects of aPD-L1 mAbs. First, in the random effects model, the three mAbs selected were considered a random sampling of the various mAbs available. This experiment was designed to answer the question: “How much of the variance in the [¹⁸F]DK222 signal can be explained by (a) the fact that there are different mAbs, or (b) these mAbs may each have different kinetics between 24-to-96 hours as opposed to lumping all of them together as active treatments?” To answer this question, the three aPD-L1 mAbs were selected as random effects, and PD-L1 mAbs vs inactive mAb, timepoints, and the overall difference of active treatments between timepoints are selected as the fixed effects. The random and fixed effects are jointly estimated in a mixed linear regression model and measure of heterogeneity is defined by “intraclass correlation coefficient (ICC)”.

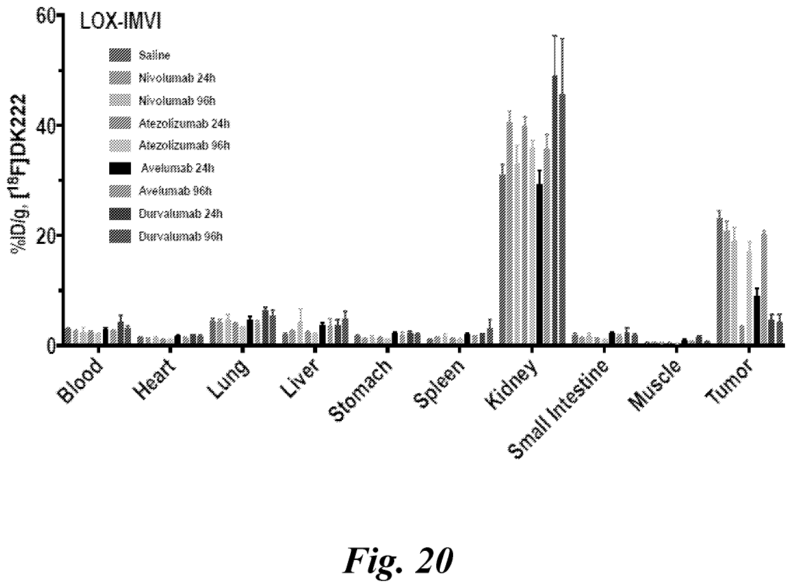

ICC quantifies the fraction (or percentage) of the total variance due to different active mAbs. The ICC can range between 0 to 1 (0 to 100%) and the larger the ICC, the greater is the variation of PD and PK to be expected amongst various aPD-L1 mAbs. The ICC of the random PD-only effect model in LOX-IMVI tumors is 0.23 (vs no random effects p-value=8.9×10⁻⁵) indicating that 23% of variance in [¹⁸F]DK222 signal (% ID/g) comes from differences in PD-L1 occupancy. The ICC of the random PD-PK effect model is 0.36 (vs no random effects p-value=3×10⁻⁶; vs random PD model p-value=0.0014) indicating that 36% of the variance in the [¹⁸F]DK222% ID/g comes from the different PD and PK of different mAbs in the tumor bed. Similarly, in the MDAMB231 tumor model, the ICC of the random PD-only effect model is 0.54 (vs no random effects p-value=0). The ICC of the random PD-PK effect model is 0.77 (vs no random effects p-value=0; vs random PD model p-value=0) indicating that 77% of the variance in the MDAMB231% ID/g comes from the different PD and PK of different mAbs in the tumor bed (FIG. 20).

Second, in the fixed effects model, each of the three fixed PD-L1 mAbs are thought of as specific choices to be compared against each other. The question: “Which one of Atezolizumab, Avelumab, or Durvalumab, specifically engages PD-L1 more effectively over time?” was investigated. To answer this pragmatic question, each PD-L1 mAb (specific saturation PD), each time point (overall PK, 24 and 96 h), and each mAb*time combination (mAb-specific PK) were considered a fixed effect, and were estimated together in an ordinary linear regression model. The results of this analysis are given as (a) the difference in accessible PD-L1 levels (% ID/g) for each of the PD-L1 mAbs at 24 h vs Nivolumab, and (b) the difference in accessible PD-L1 levels at 96 h vs 24 h for a specific mAb as compared to 96-to-24 hour difference in Nivolumab.

[¹⁸F]DK222 uptake in LOX-IMVI tumors and tissues of mice treated with different mAbs and timepoints is shown in FIG. 6C. The mean LOX-IMVI tumor % ID/g at 24 hours was high (approximately 20% ID/g) in Nivolumab control and changes very little between 24 to 96 hours (FIG. 6C). In contrast, at 24 h, all PD-L1 mAbs had a significantly lower mean tumor % ID/g than Nivolumab. The accessible PD-L1 levels at 96 h were 60 to 80% higher in Atezolizumab and Avelumab groups than those observed at 24 h of treatment (P<0.001). Accessible PD-L1 levels for Durvalumab, however, were reduced by 70% or more, and were similar at 96 h vs 24 h suggesting a longer-term engagement of PD-L1 by Durvalumab. These observations were validated in MDAMB231 xenograft model (FIG. 6D).

In sum, the random and fixed effects models reveal that PD-L1 mAbs have differential PK and PD in the tumor bed that influence accessible PD-L1 levels over time. Furthermore, these results clearly demonstrate the potential of PET to quantify accessible target levels to gain insights into pharmacological activity of mAbs at the tumor site.

### 1.2 Discussion

mAbs conjugated with radionuclides are routinely used to gain insights into their biodistribution and target expression. Nearly 26 such agents are in clinical trials. De Vries et al., 2019. A variety of mAbs, mAb-conjugates, and small proteins have been developed to detect PD-L1 expression. Josefsson et al., 2015; Maute et al., 2015; Chatterjee et al., 2016; Truillet et al., 2017; De Silva et al., 2018; Jagoda et al., 2019; Vento et al., 2019; Wissler et al., 2019; Hettich et al., 2016; Donnelly et al., 2018.

Recently, studies with Zr-89-labelled Atezolizumab have highlighted the potential of PET to quantify intra- and inter-tumor heterogeneity in PD-L1 expression. Bensch et al., 2018. In spite of those advances, there is a need for imaging agents that provide high contrast images and are compatible with a standard clinical workflow. Such high-contrast images are often observed with peptides and low molecular weight PET agents.

The presently disclosed subject matter demonstrates, in part, that that [¹⁸F]DK222 peptide exhibits PK and biodistribution features distinct from that of reported PD-L1 imaging agents. Bensch et al., 2018; Maute et al., 2015; Chatterjee et al., 2016; Truillet et al., 2017; De Silva et al., 2018; Jagoda et al., 2019; Donnelly et al., 2018; Lesniak et al., 2019; Heskamp et al., 2019; Ehlerding et al., 2019; Kikuchi et al., 2017; Chatterjee et al., 2017; Broos et al., 2017; Josefsson et al., 2016; Natarajan et al., 2015; Heskamp et al., 2015.

Moreover, [¹⁸F]DK222 possess all the salient features required for routine clinical use: 1) high affinity and specificity to quantify the dynamic changes in PD-L1 levels; 2) tractable PK compared to reported protein-based imaging agents and low non-specific accumulation in normal tissues to allow its use across many tumor types; 3) suitable image contrast within 60 min of radiotracer administration, to fit within the standard clinical workflow; and 4) human dosimetry estimates similar to other conspicuous PET imaging agents such as those used to detect prostate-specific membrane antigen and chemokine receptor 4. Szabo et al., 2015; Herrmann et al., 2015.

Radiolabeled mAb accumulation in the tumors could be indicative of tumor response to therapy. [⁸⁹Zr]Atezolizumab signal in the tumors acquired after multiple days of radiotracer injection was found to be a better predictor of tumor response to Atezolizumab therapy than IHC and RNA sequencing-based predictive biomarkers. Bensch et al., 2018. In the ever-expanding PD-1/PD-L1 therapeutic development arena, however, [⁸⁹Zr]mAb imaging for head-to-head comparisons between mAb therapeutics, or to gain deeper insights into differences in their distribution and activity at the tumor is impractical for clinical translation. Yu et al., 2019.

It is demonstrated herein that radiopharmaceuticals with high affinity and faster pharmacokinetics, such as [¹⁸F]DK222, can be useful beyond baseline PD-L1 level quantification and for therapy guidance. The potential of such measurements to evaluate the in situ pharmacological activity of different aPD-L1 mAbs is shown by discovering the prolonged target engagement by Durvalumab compared to other aPD-L1 mAbs in the preclinical models employed. Importantly, these PD measures encapsulate multiple factors that influence antibody concentrations including PD-L1 levels and turnover, complex serum and tumor kinetics (or fate) of those mAbs at the tumor, and tumor-intrinsic parameters such as high interstitial pressure and poor vascularity, that impede mAb penetration and accumulation. Moreover, those 3 mAbs exhibit distinct PK [Atezolizumab (isotype IgG1κ; K_{D}, 0.4 nM; t_{1/2}, 27 days), Avelumab (IgG1λ, 0.7 nM, 6.1 days), and Durvalumab (IgG1η, 0.022 nM, 18 days)]and the tumor residence kinetics of these mAbs do not mirror circulating half-life profiles but reflect mAb affinity for PD-L1. Tan et al., 2017.

The approach and findings of the current study also have potential implications for improving treatment regimens and in drug development and evaluation. Predictive computational models are routinely used in clinical development and dosing of mAbs. Agoram, 2007; Agoram, 2009. Personalized cancer treatment based on those mechanistic models, however, may be biased due to the preclinical information used and the lack of translatability between preclinical experiments and patients. Non-invasive measurements shown here could form that bridge. Additionally, the emergence of a variety of next generation mAb therapeutics, such as probodies that are specifically activated in the TME, Giesen et al., 2019, and multi-specific mAb conjugates that enable higher-avidity binding by promoting simultaneous binding to multiple targets, Lan et al., 2018, are likely to exhibit PK that differ from the traditional in silico models, and will require new approaches such as measuring pharmacodynamic effects at the tumor, that take account of their pharmacological activity at the tumor.

It is important to note that DK222 is a more hydrophilic peptide and significantly differs in in vivo distribution from other reported peptides, including WL12. WL12 shows high liver, kidney and non-specific accumulation in several tissues due to lipophilicity. The tumor-to-blood and tumor-to-muscle ratios for [⁶⁴Cu]WL12 for MDAMB231 tumors at 120 min after radiotracer injection were 12.9+2.1 and 2.72+0.45, respectively. In contrast the tumor-to-blood and tumor-to-muscle ratios for [¹⁸F]DK222 are 35.69+3.89 and 9.45+0.51, respectively (Specific activity: 250 mCi/tmole). Other than tumors, high radioactivity uptake is seen only in kidney an organ involved with clearance of [¹⁸F]DK222. That high tumor uptake and low background tissue uptake results in high image contrast PD-L1 specific images.

Further, the presently disclosed DK222 also is radiolabeled differently than the previously reported peptide analogs (i.e., international PCT patent application publication no. WO/2017/201 111 (PCT/US2017/033004), for PET-IMAGING IMMUNOMODULATORS, to Donnelly et al., published Nov. 23, 2017, which is incorporated herein by reference in its entirety). The lysine s-amine of DK221 is used for bifunctional chelator conjugation using the NHS ester method and requires milder conditions than the methods used previously, which did all the conjugations on the aminoacetamide end which would require incorporating the glycine during the peptide synthesis or using harsh conditions for conjugation.

Use of [¹⁸F]AlF-based radiolabeling facilitates a one-step radiolabeling procedure that can be accomplished within 60 min without requiring special equipment. AlF radiolabeling strategy also keeps the hydrophilicity of the molecule intact which is required for the high contrast images seen. Although AlF radiolabeling has been reported previously with NOTA or NODAGA analogs, we have observed more robust and consistent radiofluorination with the described NODA analog further providing an advantage over existing radiolabeling strategies applied for the development of PD-L1 imaging agents.

Replacing AlF with Pyl radiolabeling strategy ([¹⁸F]DK221-Py) changes the PK of the molecule and results in poor image contrast underscoring the importance of aluminum fluoride radiolabeled NODA conjugated DK221 as the optimal choice for the development of described imaging agent. It is possible that one could further modulate the in vivo pharmacokinetics and biodistribution of [¹⁸F]DK221-Py by the addition of PEG or other linkers to achieve desired contrast.

### 1.3 Materials and Methods

1.3.1 Chemicals. DK221 was custom synthesized by CPC Scientific (Sunnyvale, Calif.) with >95% purity. (2,2′-(7-(4-isothiocyanatobenzyl)-1,4,7-triazonane-1,4-diyl)diacetic acid) (NCS-MP-NODA) was purchased from CheMatech Macrocycle Design Technologies (catalog #C110; Dijon, France). All other chemicals were purchased from Sigma-Aldrich or Fisher Scientific.

1.3.2. Cell culture reagents and antibodies. All cell culture reagents were purchased from Invitrogen (Grand Island, N.Y.). The aPD-L1 mAbs (Atezolizumab, Avelumab, and Durvalumab) and aPD-1 mAbs (Nivolumab and Pembrolizumab) were purchased from Johns Hopkins School of Medicine Pharmacy.

1.3.3 Synthesis of DK222. DK221 is a 14 amino acid cyclic peptide with the sequence Cyclo-(-Ac-Tyr-NMeAla-Asn-Pro-His-Glu-Hyp-Trp-Ser-Trp(Carboxymethyl)-NMeNle-NMeNle-Lys-Cys-)-Gly-NH₂. It was previously reported as peptide 6297. Miller et al., 2016. The NODA conjugated analog of DK221 (Cyclo-(-Ac-Tyr-NMeAla-Asn-Pro-His-Glu-Hyp-Trp-Ser-Trp(Carboxymethyl)-NMeNle-NMeNle-Lys(NODA_NCS[¹⁸F]AlF)-Cys-)-Gly-NH₂) was prepared as follows. To a stirred solution of DK221 (4.0 mg, 2.04 μmoles) in a 20 mL vial in Dimethylformamide (1.0 mL) was added Diisopropylethylamine (5.0 μL) followed by NCS-MP-NODA (1.6 mg, 4.07 μmoles). The reaction mixture was stirred for 4 h at room temperature. The reaction mixture was purified on a reversed phase high performance liquid chromatography (RP-HPLC) system using a semi-preparative C-18 Luna column (5 mm, 10×250 mm Phenomenex, Torrance, Calif.). The HPLC conditions for purification were 50-90% methanol (0.1% trifluoroacetic acid) and H₂O (0.1% trifluoroacetic acid) in 30 min at a flow rate of 5 mL/min. The desired DK222 was collected at 15.5 min, solvent evaporated, residue reconstituted in deionized water, and lyophilized to powder in 65% yield. The purified DK222 was characterized by matrix assisted laser desorption/ionization time-of-flight mass spectrometry (MALDI-TOF MS). Calculated [M+H]⁺: 2348.68, Observed: 2349.06 (FIG. 10 and FIG. 11).

1.3.4 Synthesis of [¹⁹F]DK222. A solution of 2 mL NaF (1M, in 0.5M NaOAc, pH 4) and 2 mL AlCl₃ (0.2M, in 0.5M NaOAc, pH 4) was stirred in a 20 mL vial for 10 min at room temperature. To this vial, a prepared solution of DK222 (5 mg, 2.12 μmoles, in 200 μL of acetonitrile and 100 μL 0.5M NaOAc, pH 4) was added and heated at 110° C. for 35 min. The reaction mixture was cooled to room temperature and evaporated to half of total volume. Reaction mixture was loaded onto three-in-series pre-activated Sep-Pak plus C18 Cartridges and washed subsequently with 5 mL water (x5). The desired [¹⁹F]DK222 was eluted with 50% acetonitrile in water (5 mL×5). The collected fractions were combined, concentrated under rotavap, reconstituted in 20% acetonitrile in water, and lyophilized to form an off white powder in 80% yield. The resulting pure product was characterized by MALDI-TOF. Calculated [M=H]⁺:2392.65, observed: 2393.03. The pure [¹⁹F]DK222 complex was then used to optimize RP-HPLC conditions, as a standard for radiolabeling, and for PD-L1 and PD-1 competition binding assay. The HPLC chromatograms and mass spectrometry analysis of [¹⁹F]DK222 are shown in FIG. 10 and FIG. 11.

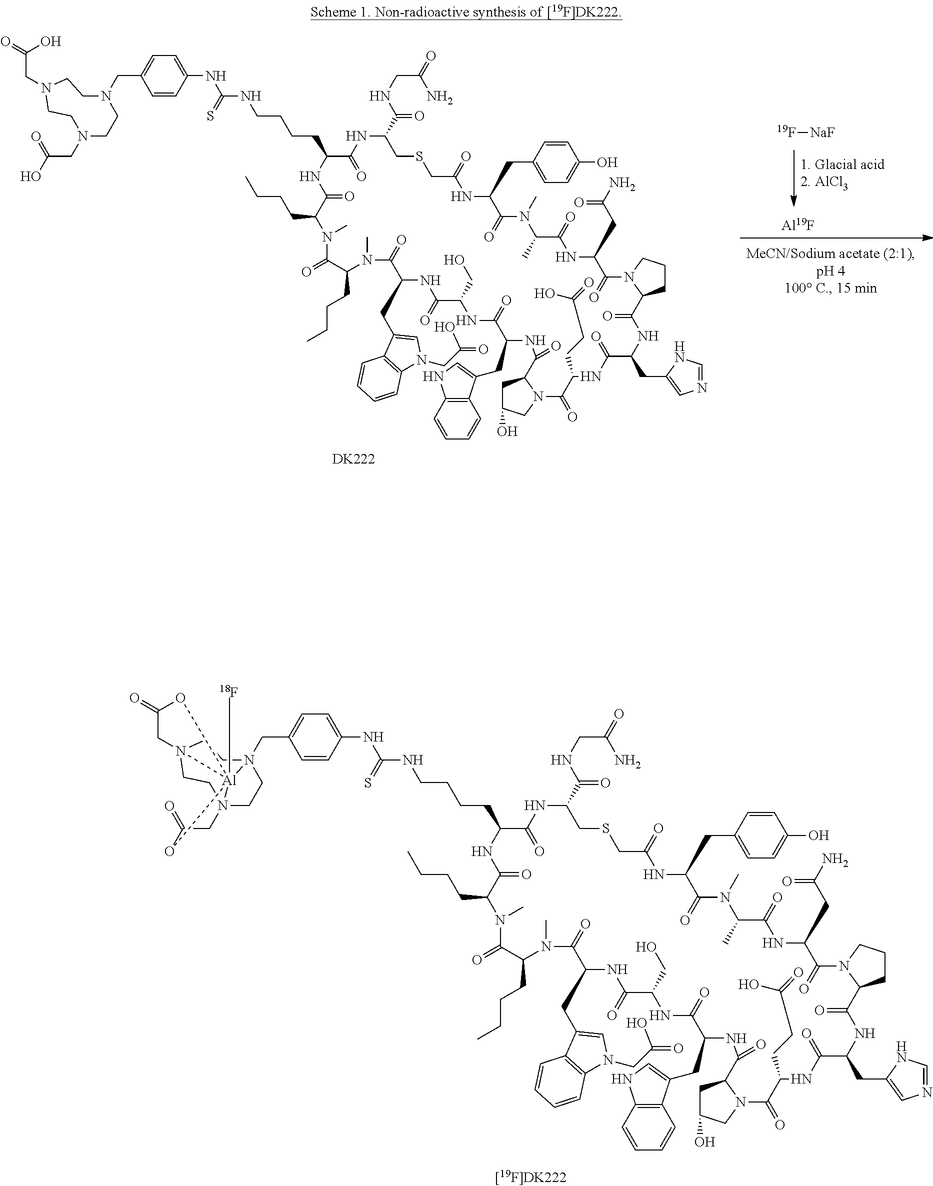

1.3.5. MALDI-TOF analysis. MALDI-TOF spectra of DK222 and its precursors were obtained on a Voyager DE-STR MALDI-TOF available at the Johns Hopkins University Mass Spectrometry core facility. Briefly, samples were equilibrated in water with 0.1% TFA using Amicon Ultra-15 centrifugal filter units (catalog UFC901008). Samples were mixed (1:2 dilution) with 10 mg/ml sinapinic acid (3,5-dimethoxy-4-hydroxycinnamic acid) matrix dissolved in 40% acetonitrile and 0.1% TFA. 1 μL of those samples was spotted in quadruplet on a MALDI plate (Applied Biosystems) and allowed to air dry, followed by spectra acquisition using optimized instrument settings. Data were analyzed using Applied Biosystems Data Explorer software version 4.8.

1.3.6 [⁸F]DK222 Radiopharmaceutical preparation. The [¹⁸F] fluoride (non-carrier added) received from the JHU PET Center cyclotron was trapped on a preconditioned Chromafix 30-PS-HCO₃ cartridge. The cartridge was subsequently washed with metal-free water (5 mL). ¹⁸F was eluted from the cartridge with 100 μL of 0.4 M KHCO3. The pH of the solution was adjusted to approximately 4 with 10 μL of metal-free glacial acetic acid, followed by the addition of 20 μL of 2 mM AlCl_{3.6}H₂O in 0.1M sodium acetate buffer (pH 4). The resulting solution was incubated at room temperature for 2-4 min to form Al¹⁸F complex. The precursor DK222 (approximately 100 micrograms, 42 nmoles) was dissolved in 300 μL of 2:1 solution of acetonitrile and NaOAc (0.1M, pH 4) and then added to the vial containing Al¹⁸F. The resulting reaction mixture was heated at 110° C. for 15 min. Then, the reaction vial was cooled to room temperature and diluted with 400 μL DI Water. The obtained aqueous solution containing the radiolabeled product was purified on a RP-HPLC system (Varian ProStar) with an Agilent Technology 1260 Infinity photodiode array detector (Agilent Technologies, Wilmington, Del.). A semi-preparative C-18 Luna column (5 mm, 10×250 mm Phenomenex, Torrance, Calif.) was used with a gradient elution starting with 50% Methanol (0.1% TFA) and reaching 90% of Methanol in 30 min at a flow rate of 5 mL/min with water (0.1% TFA) as co-solvent. The radiolabeled product, [¹⁸F]DK222, eluted at a retention time of approximately 16.2 min was collected, evaporated under high vacuum, formulated with saline containing 10% EtOH, sterile filtered, and used for in vitro and in vivo evaluation.

The radiochemical purity, chemical identity, and in vitro stability HPLC chromatograms are shown in FIG. 11A-FIG. 11C.

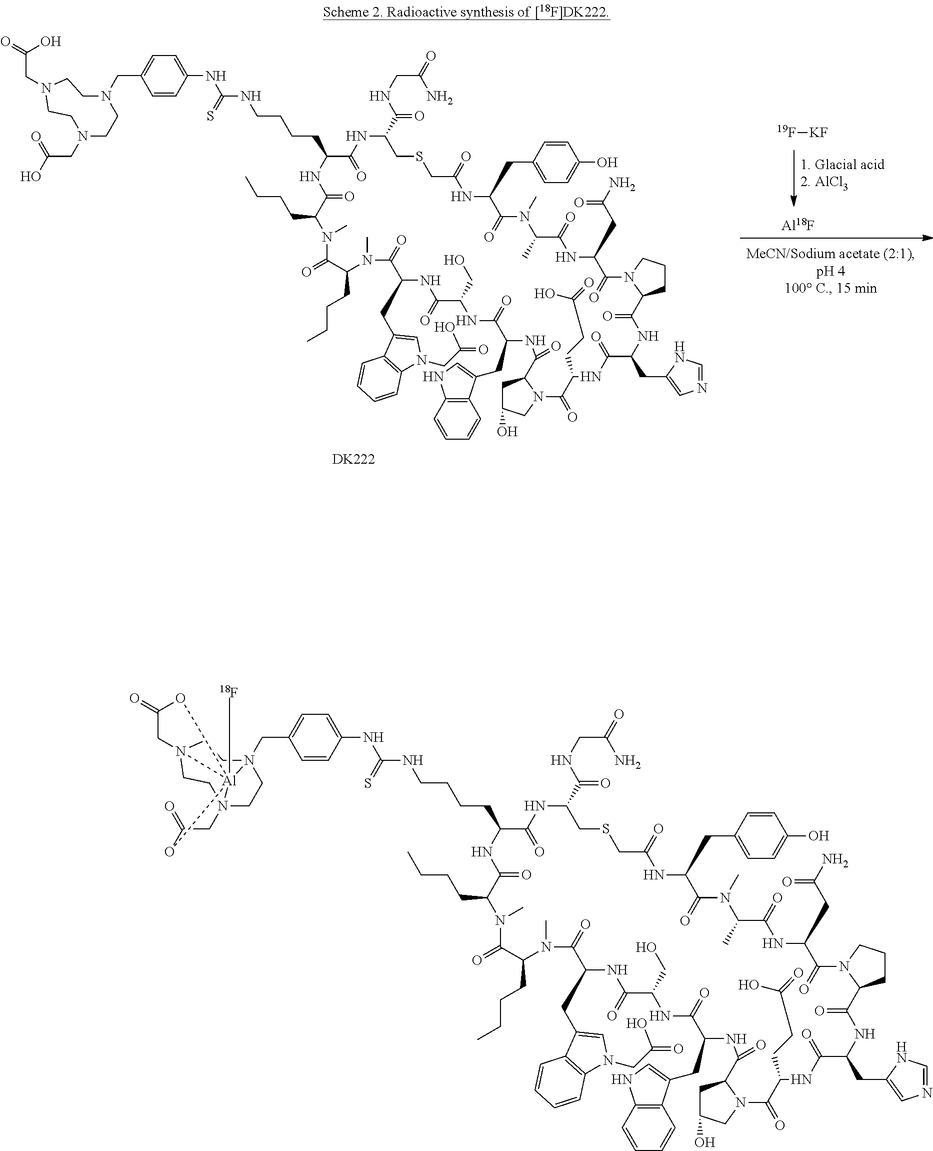

1.3.7 Cell culture. Seven cell lines were used for in vitro and in vivo evaluation: MDAMB231 and SUM149 (triple negative breast cancer), LOX-IMVI, MeWo and A375 (melanoma), CHO, and CHO cells constitutively expressing PD-L1 (hPD-L1). The MDAMB231, MeWo, A375, and CHO cells were purchased from the American Type Culture Collection and cultured as recommended. The CHO cells constitutively expressing PD-L1 (hPD-L1) were generated in our laboratory and cultured as previously described. Chatterjee et al., 2016. The SUM149 cell line was obtained from Dr. Stephen Ethier and LOX-IMVI cell line was obtained from NCI developmental therapeutic program. All cell lines were authenticated by STR profiling at the Johns Hopkins genetic resources facility. The SUM149 cells were maintained in Ham's F-12 medium with 5% ^{F}BS. 1% P/S and 5 μg/mL insulin, and 0.5 μg/mL hydrocortisone. All cell lines were cultured in the recommended media in an incubator at 37° C. in an atmosphere containing 5% CO₂. Human embryonic kidney (HEK) 293F cells (Thermo Life Technologies) used for protein expression were maintained in suspension in FreeStyle 293 expression medium (Thermo Life Technologies) containing 0.01% penicillin-streptomycin (Gibco) at 37° C. with 5% ambient CO₂.

1.3.8 Detection of PD-L1 expression by flow cytometry. Cells were evaluated for PD-L1 surface expression by direct staining of 2×10⁵ cells in 100 μL PBS with Cy5-Atezolizumab, for 30 min at 4° C. Cy5-Atezolizumab was prepared as described previously. Kumar et al., 2019. Cells were then washed and analyzed for mean fluorescence intensity (MFI) by flow cytometry. Adherent cells were detached using enzyme-free cell dissociation buffer (Thermo Fisher Scientific, Waltham, Mass.). 1.3.9 In vitro binding assays with [¹⁸F]DK222. In vitro binding of [¹⁸F]DK222 to hPD-L1 MDAMB231, MeWo, A375, Sum149, and CHO cells was determined by incubating 1×10⁶ cells with approximately 0.1 ρCi of [¹⁸F]DK222 in the presence, or absence, of 1 μM of DK222 or 60 nM mAbs for 30 min at 4° C. After incubation, cells were washed three times with ice cold PBS containing 0.1% Tween20 and counted on an automated gamma counter (1282 Compugamma CS, Pharmacia/LKBNuclear, Inc., Gaithersburg, Md.). To demonstrate PD-L1-specific binding of [¹⁸F]DK222, blocking was performed with 1 μM of unmodified peptide DK221. All cell radioactivity uptake studies were performed in quadruplicate for each cell line and repeated three times.

1.3.10. In vivo studies. All mouse studies were conducted through Johns Hopkins University Animal Care and Use Committee (ACUC) approved protocols. Xenografts were established in five-to-six-week-old, male or female, non-obese, diabetic, severe-combined immunodeficient gamma (NSG) mice obtained from the Johns Hopkins University Immune Compromised Animal Core. huPBMC mice were purchased from Jackson (JAX) laboratories and used for experiments as-is.

1.3.11 Xenograft models. Mice were implanted in the rostral end with MDAMB231 (2×10⁶, orthotopic), SUM149 (5×10⁶, orthotopic), LOX-IMVI (5×10⁶, intradermal), MeWo (5×10⁶, intradermal), or A375 (2×10⁶, intradermal) cells. Cells were inoculated in the opposite flanks if two cell lines were used with cell line expressing high PD-L1 on right side of the mouse. Mice with tumor volumes of 200-400 mm³ were used for treatment, imaging, or biodistribution experiments.

1.3.12 PET-CT imaging of mouse xenografts. To determine the in vivo distribution and pharmacokinetics of [¹⁸F]DK222, PET images were acquired at multiple time points. Mice with MDAMB231 tumors were injected with ˜200 Ci (7.4 mBq) of [¹⁸F]DK222 in 200 μL of saline intravenously (n=3) and anesthetized under 3% isofluorane prior to being placed on the scanner. PET images were acquired at 15, 60, and 120 min after radiotracer injection in two bed positions at 5 min/bed in an ARGUS small-animal PET/CT scanner (Sedecal, Madrid, Spain) as described. Lesniak et al., 2016. A CT scan (512 projections) was performed at the end of each PET scan for anatomical co-registration. The PET data were reconstructed using the two-dimensional ordered subsets-expectation maximization algorithm (2D-OSEM) and corrected for dead time and radioactive decay. The % ID per cc values were calculated based on a calibration factor obtained from a known radioactive quantity. Image fusion, visualization, and 3D rendering were accomplished using Amira 6.1® (FEI, Hillsboro, Oreg.). PET or PET/CT images were acquired at 60 min (one or two beds, 5 min/bed) after radiotracer injection in all other tumor models.

For all other PET imaging studies reported herein, mice received approximately 200 μCi (7.4 mBq) of [¹⁸F]DK222 in 200 μL of saline intravenously and PET or PET/CT images were acquired at 60 min after injection at 5 min/bed in an ARGUS small-animal PET/CT scanner.

1.3.13 Ex vivo biodistribution. To validate imaging studies, ex vivo biodistribution studies were conducted in mice harboring human tumor xenografts (MDAMB231, Sum149, LOX-IMVI, and MeWo), as described. Lesniak et al., 2016. Mice harboring MDAMB231 tumors were injected intravenously with 50 μCi (1.85 MBq) of [¹⁸F]DK222 and tissues were harvested at 5, 30, 60, 120, 240, or 360 min after injection. Biodistribution studies were conducted at 60 min after [¹⁸F]DK222 injection in all other tumor models. For the blocking study, 2 mg/kg (50 μg) of unmodified peptide was co-injected with the radiotracer. To facilitate radiation dosimetry calculations, tissues harvested included tumors, blood, thymus, heart, lung, liver, stomach, pancreas, spleen, adrenals, kidney, small and large intestines, ovaries, uterus, muscle, femur, brain, and bladder. Harvested tissues were weighed and counted in an automated gamma counter (Perkin Elmer—2480 Automatic Gamma counter—Wizard2 3” Wallac, Waltham, Mass.), and the percentage of injected dose per gram of tissue (% ID/g) values were calculated based on signal decay correction and normalization to external [¹⁸F] standards measured in triplicate. Biodistribution data shown is mean±the standard error of the mean (SEM).

For all other biodistribution studies reported herein, mice received ˜50 Ci (1.85 mBq) of [¹⁸F]DK222 in 200 μL of saline intravenously and biodistribution studies were conducted at 60 min after [¹⁸F]DK222 injection. Selected tissues (tumors, blood, heart, lung, liver, spleen, kidney, small intestines, and muscle) were collected, weighed, counted, and their % ID/g values calculated.

1.3.14 aPD-1 mAb dosing studies. huPBMC mice acquired from JAX labs were implanted subcutaneously with 2×10⁶ A375 cells in the rostral end. Seven days after cell inoculation (average tumor volume=80±15 mm³), mice were randomized and treated with a single 12 mg/kg dose of Nivolumab or Pembrolizumab injected intravenously (n=9/group). huPBMC mice treated with saline (n=4-5/group). or NSG mice treated with 12 mg/kg dose of Nivolumab or Pembrolizumab were used as controls. Seven days following treatment, [¹⁸F]DK222 PET scans were acquired on at least 3-5 mice per group. Mice were used for biodistribution studies on day 8 after treatment and 24 h after PET imaging, and data were processed as described. Harvested tumors were cut in half and used for flow cytometry analysis or for IHC analysis for PD-L1 and CD3.

1.3.15 Flow cytometry analysis. After ex vivo biodistribution analysis, tumors and spleen were stored in MACS Tissue Storage Solution (Miltenyi Biotec #130-100-008) overnight at 4° C. Tumors and spleens were dissociated next day following manufacturer's instructions (Miltenyi Biotec 130-095-929). Briefly, each xenograft and spleen were cut into small pieces of 3-4 mm. For each tumor, cut pieces were suspended in 2.5 mL RPMI-1640 media containing 100 μL Enzyme H, 50 μL Enzyme R, and 12.5 μL Enzyme A. For each spleen, cut pieces were suspended in 2.5 mL FACS buffer containing 50 μL Enzyme D and 15 μL Enzyme A. Recommended programs were run on gentleMACS™ Octo Dissociator with Heaters (Miltenyi Biotec #130-096-427) for tumor and spleen. A short centrifugation step was performed to collect the sample material at the bottom of the tube. Sample was resuspended and passed through a strainer (70 μm for tumor and 30 μm for spleen), centrifuged 300×g for 7 minutes, supernatant was discarded, and cells were resuspended in 2.5 mL FACS buffer. Cells were counted and 1×10⁶ cells were resuspended in 100 μL Live/Dead aqua solution (ThermoFisher #L34965, 2 μL reconstituted with 2 mL PBS) in a 96 well plate. The cells were incubated for 15 min (dark, RT) and washed with 150 μL PBS. Fc blocking was performed with Biolegend Tru Stain (#422301 Fc Block (1 μL in 100 μL FACS buffer) and samples were incubated for 10 min (dark, 4° C.). After washing with 150 μL cold FACS buffer, the samples were stained with antibodies targeting markers of interest in following dilutions in 100 μL FACS buffer:

| Marker | Fluorophore | Clone | Supplier | Catalog No | Dilution |
|---|---|---|---|---|---|
| CD14 | FITC | M5E2 | BD | 555397 | 1:50 |
| HLA-DR | BV-605 | G46-6 | BD | 562845 | 1:500 |
| PD-L1 | BV421 | MIH1 | BD | 563738 | 1:40 |
| PD-1 | PerCP-ef710 | MIH4 | ThermoFisher | 46-9969-42 | 1:100 |
| CD45 | BV650 | HI30 | BD | 563717 | 1:100 |
| CD45 | apc-cy7 | UCHT1 | BD | 300425 | 1:100 |
| CD8 | PE-cy7 | HIT8a | BioLegend | 300914 | 1:100 |
| Igg1k | BV421 | isotype | BioLegend | 400157 | 1:40 |

They were incubated for 15 min (dark, RT), washed with 200 μL FACS buffer, and fixed with 200 μL Fix/Perm (eBio Foxp3 staining kit #00-5523-00: 1 vol Fix-Perm concentrate with 3 vols Diluent). Samples were resuspended in 500 μL FACS buffer the next day on LSR II. All data were analyzed on FlowJo v10.4.1.

1.3.16 Immunohistochemical analysis. Immunochemical analysis for PD-L1 was performed as described previously using clone (E1L3N®) XP® Rabbit Anti-Human PD-L1 (Cell Signaling, 13684, Dilution: 1:250). Gniadek et al., 2017. Immunohistochemical staining for CD3 was performed using Polyclonal Rabbit Anti-Human CD3, (Dako/Agilent, A0452, Dilution: 1:100) by NDBio Inc., (Baltimore) using clone.

1.3.17 aPD-L1 mAb dosing studies. To determine the effect of antibody dose on accessible PD-L1 levels in the tumor, LOX-IMVI tumor-bearing mice were treated with a single intravenous bolus dose of Atezolizumab (0.3 or 20 mg/Kg) and 24 hours later mice were used for imaging (n=3-4) and biodistribution (n=6-8) studies.

To determine temporal changes in accessible PD-L1 levels in the tumor after treatment with PD-L1 therapeutics, LOX-IMVI tumor-bearing mice were treated with a single intravenous bolus dose of Atezoluzumab, Avelumab, or Durvalumab (1 mg/kg) and imaging (n=3) and biodistribution studies (n=8-18) were conducted at 24 and 96 h after antibody treatment. Anti PD-1 antibody Nivolumab (1 mg/kg) and saline were used as controls. Mice treated for 24 h with therapeutic mAbs, or saline as a control, were injected with 200 μCi of [¹⁸F]DK222 in 200 μL of saline intravenously, and PET images were acquired 1 hour after the injection of the radiotracer. Due to the many number of groups and mice involved, saline and Nivolumab-treated controls were included in every experiment, and data from multiple experiments were pooled. Study was repeated in MDAMB231 tumor-bearing mice with only biodistribution measurements.

1.3.18 Data analysis. Statistical Analyses were performed using Prism 8 Software (GraphPad Software, La Jolla, Calif.). Unpaired Student's t-test, one or two-way ANOVA were utilized for column, multiple column, and grouped analyses respectively. Data represent mean±SEM. P-values<0.05 were considered statistically significant.

### 1.4 Synthesis

1.4.1 DK221 Analogs

1.4.1.1 DK222 (DK221-NODA)

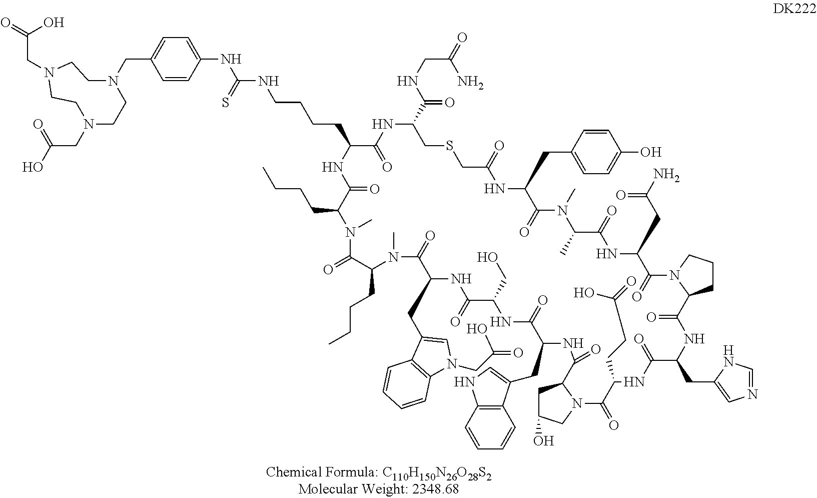 [SMILES: CCCC[C@H]1C(=O)N(C)[C@@H](CCCC)C(=O)N[C@@H](CCCCNC(=S)Nc2ccc(CN3CCN(CC(=O)O)CCN(CC(=O)O)CC3)cc2)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](Cc2cnc[nH]2)C(=O)N[C@@H](CCC(=O)O)C(=O)N2C[C@H](O)C[C@H]2C(=O)N[C@@H](Cc2c[nH]c3ccccc23)C(=O)N[C@@H](CO)C(=O)N[C@@H](Cc2cn(CC(=O)O)c3ccccc23)C(=O)N1C]

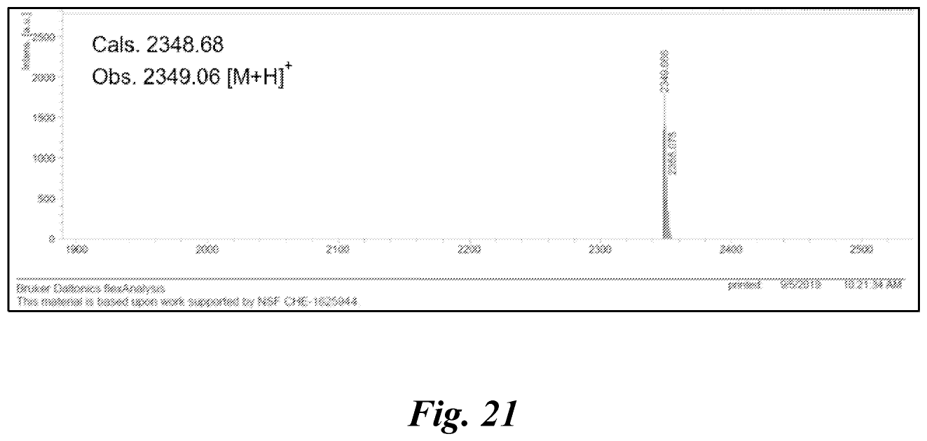

1.4.1.1.A Procedure: To a stirred solution of DK221 (4.0 mg, 2.04 μmoles) in a 20 mL vial in Dimethylformamide (1.0 mL) was added Diisopropylethylamine (5.0 μL) followed by NCS-MP-NODA (1.6 mg, 4.07 μmoles). The reaction mixture was stirred for 4 h at room temperature. The reaction mixture was purified on a reversed phase high performance liquid chromatography (RP-HPLC) system using a semi-preparative C-18 Luna column (5 mm, 10×250 mm Phenomenex, Torrance, Calif.). The HPLC conditions for purification were 50-90% methanol (0.1% trifluoroacetic acid) and H₂O (0.1% trifluoroacetic acid) in 30 min at a flow rate of 5 mL/min. The desired DK222 was collected at 15.5 min, solvent evaporated, residue reconstituted in deionized water, and lyophilized to powder in 65% yield. The purified DK222 was characterized by matrix assisted laser desorption/ionization time-of-flight mass spectrometry (MALDI-TOF MS). Calculated [M+H]+: 2348.68, Observed: 2349.06. The MALDI-TOF MS of DK222 is shown in FIG. 21.

1.4.1.2 DK331 (DK221-Biotin)

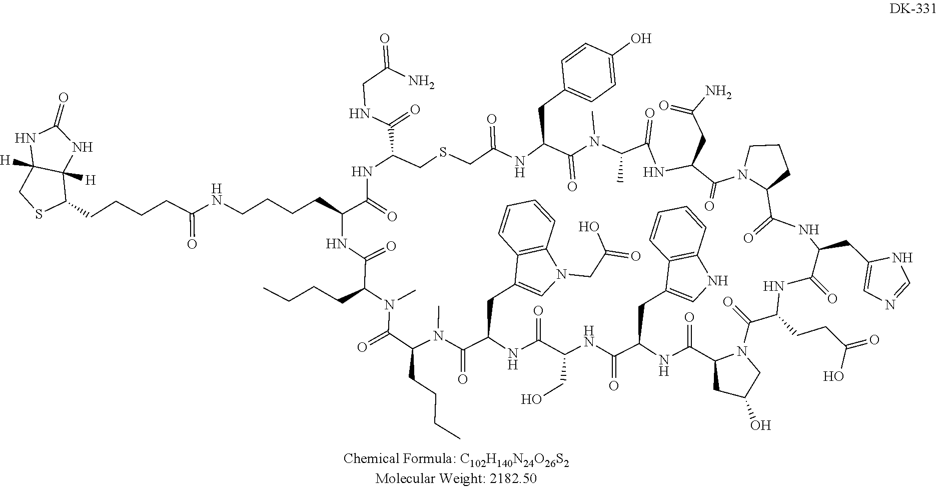 [SMILES: CCCC[C@H]1C(=O)N(C)[C@@H](CCCC)C(=O)N[C@@H](CCCCNC(=O)CCCC[C@@H]2SC[C@@H]3NC(=O)N[C@@H]32)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](Cc2cnc[nH]2)C(=O)N[C@H](CCC(=O)O)C(=O)N2C[C@H](O)C[C@H]2C(=O)N[C@H](Cc2c[nH]c3ccccc23)C(=O)N[C@H](CO)C(=O)N[C@H](Cc2cn(CC(=O)O)c3ccccc23)C(=O)N1C]

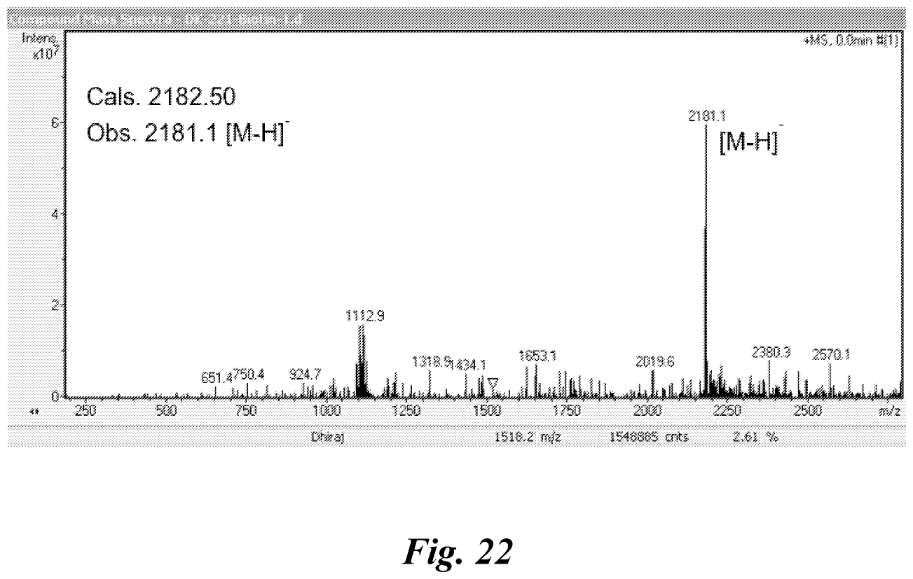

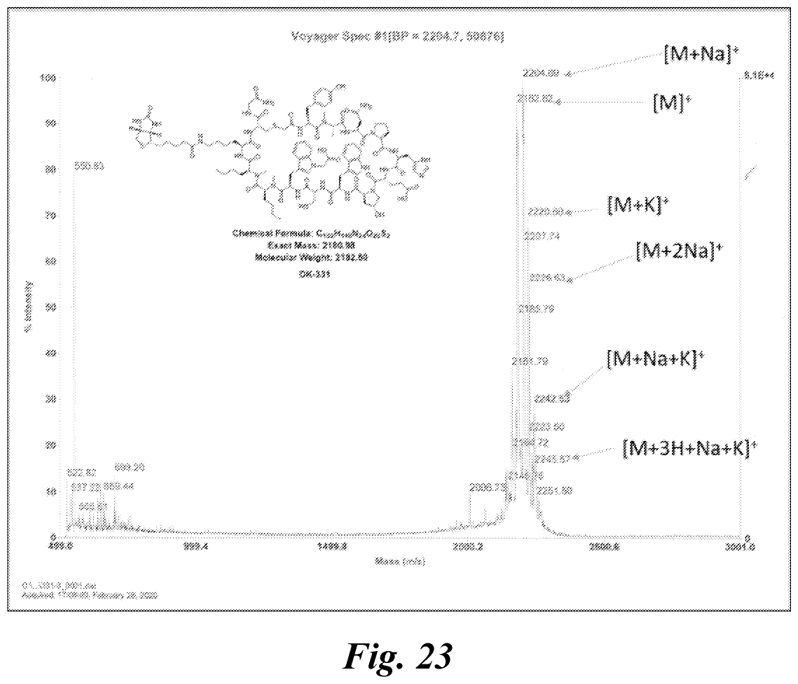

1.4.1.2.A Procedure: To a stirred solution of DK221 (5.0 mg, 2.55 μmoles) in a 20 mL vial in Dimethylformamide (1.0 mL) was added Diisopropylethylamine (5.0 μL) followed by Biotin-NHS-Ester (2.0 mg, 5.85 μmoles). The reaction mixture was stirred for 3-4 h at room temperature. The reaction mixture was purified on a reversed phase high performance liquid chromatography (RP-HPLC) system using a semi-preparative C-18 Luna column (5 mm, 10×250 mm Phenomenex, Torrance, Calif.). The HPLC conditions for purification were 20-60% acetonitrile (0.1% trifluoroacetic acid) and H₂O (0.1% trifluoroacetic acid) in 25 min at a flow rate of 5 m/min. The desired DK331 was lyophilized to powder form in 57% yield which was characterized by ESI MS. Calculated [M−H]⁻: 2182.50, Observed: 2181.1. The ESI-MS of DK331 is shown FIG. 22. The MALDI-MS of DK331 is shown in FIG. 23.

1.4.1.3. DK225 (DK221-NODAGA)

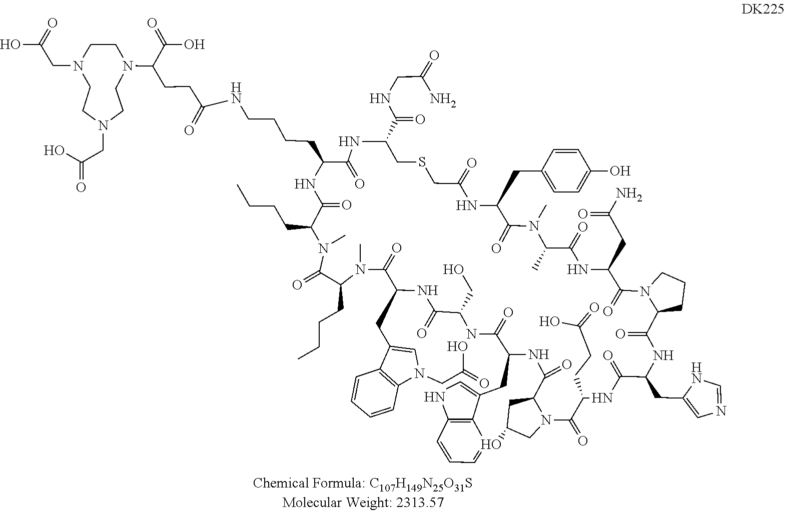

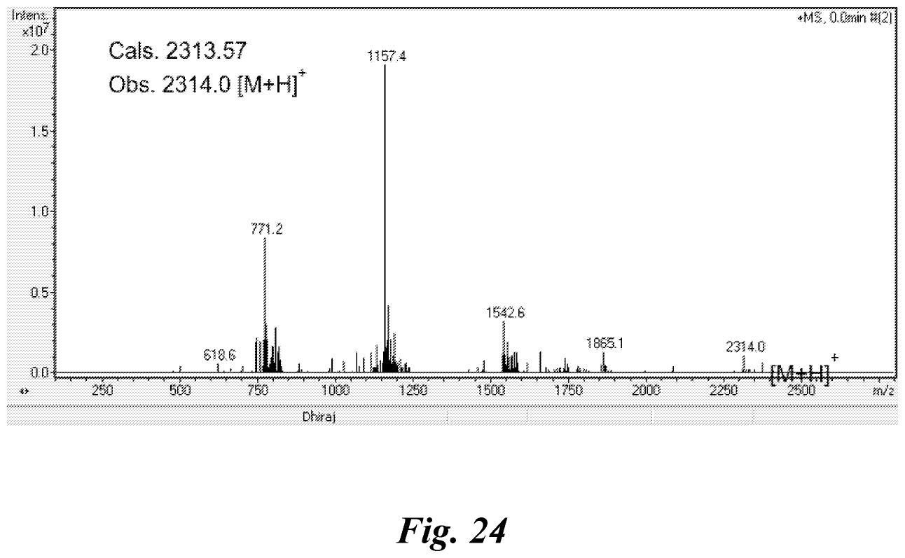

1.4.1.3.A Procedure: To a stirred solution of DK221 (4.0 mg, 2.04 μmoles) in a 20 mL vial in Dimethylformamide (1.0 mL) was added Diisopropylethylamine (5.0 μL) followed by NODAGA-NHS-Ester (2.0 mg, 2.73 μmoles). The reaction mixture was stirred for 3-4 h at room temperature. The reaction mixture was purified on a reversed phase high performance liquid chromatography (RP-HPLC) system using a semi-preparative C-18 Luna column (5 mm, 10×250 mm Phenomenex, Torrance, Calif.). The HPLC conditions for purification were 20-60% acetonitrile (0.1% trifluoroacetic acid) and H₂O (0.1% trifluoroacetic acid) in 25 min at a flow rate of 5 mL/min. The desired DK225 was lyophilized to powder form in 62% yield which was characterized by ESI MS. Calculated [M+H]⁺: 2313.57; Observed: 2314.0. The ESI-MS of DK225 is shown in FIG. 24.

1.4.1.4. DK223 (DK221-DOTA)

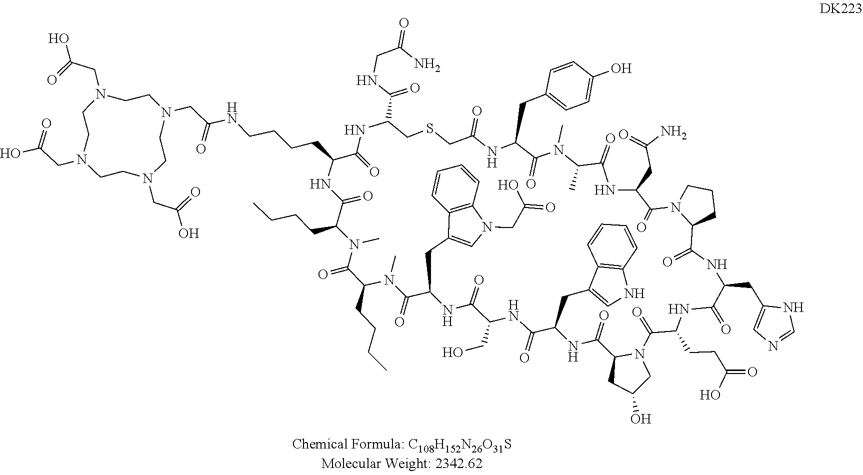

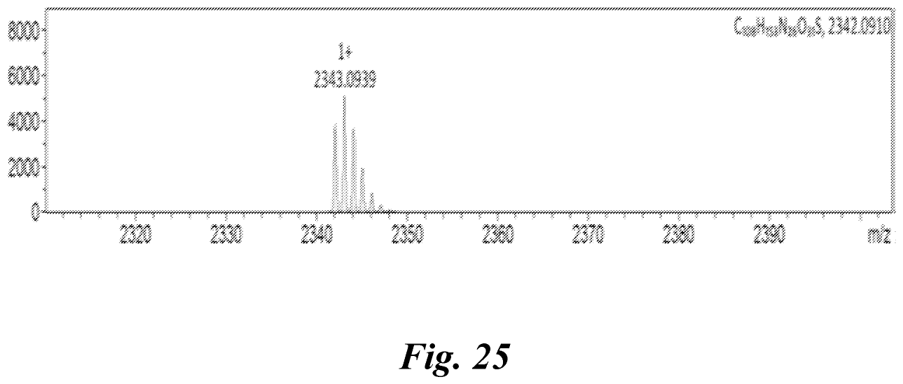

1.4.1.4.A. Procedure: To a stirred solution of DK221 (5.0 mg, 2.55 μmoles) in a 20 mL vial in Dimethylformamide (1.0 mL) was added Diisopropylethylamine (5.0 μL) followed by DOTA-NHS-Ester (2.0 mg, 2.62 μmoles). The reaction mixture was stirred for 4 h at room temperature. The reaction mixture was purified on a reversed phase high performance liquid chromatography (RP-HPLC) system using a semi-preparative C-18 Luna column (5 mm, 10×250 mm Phenomenex, Torrance, Calif.). The HPLC conditions for purification were 20-60% acetonitrile (0.10% trifluoroacetic acid) and H₂O (0.1% trifluoroacetic acid) in 25 min at a flow rate of 5 mL/min. The desired DK223 was lyophilized to powder form in 72% yield which was characterized by matrix assisted laser desorption/ionization time-of-flight mass spectrometry (MALDI-TOF MS). Calculated [M+H]+: 2342.62, Observed: 2343.09. The MALDI-MS of DK223 is shown in FIG. 25.

### 1.4.1.5. DK385 (DK221-DOTAGA)

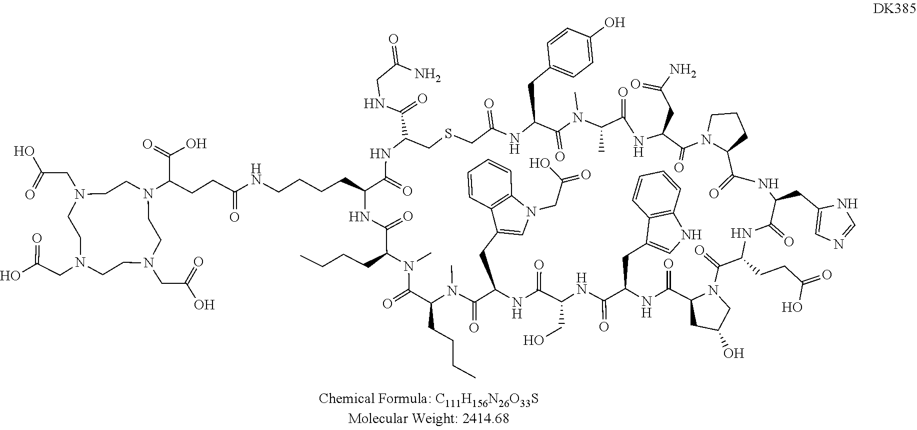

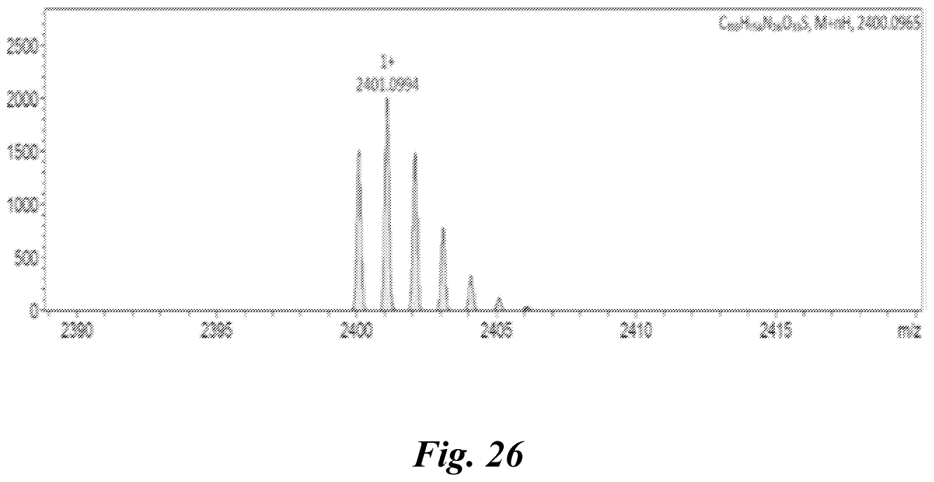

1.4.1.5.A. Procedure: To a stirred solution of DK221 (5.0 mg, 2.55 μmoles) in a 20 mL vial in Dimethylformamide (1.0 mL) was added Diisopropylethylamine (5.0 μL) followed by DOTA-GA anhydride (3.1 mg, 6.27 μmoles). The reaction mixture was stirred for 4 h at room temperature. The reaction mixture was purified on a reversed phase high performance liquid chromatography (RP-HPLC) system using a semi-preparative C-18 Luna column (5 mm, 10×250 mm Phenomenex, Torrance, Calif.). The HPLC conditions for purification were 20-60% acetonitrile (0.1% trifluoroacetic acid) and H₂O (0.1% trifluoroacetic acid) in 25 min at a flow rate of 5 m/min. The desired DK385 was lyophilized to powder form in 61% yield which was characterized by matrix assisted laser desorption/ionization time-of-flight mass spectrometry (MALDI-TOF MS). Calculated [M+H]⁺: 2400.65, Observed: 2401.09. The MALDI-MS of DK385 is shown in FIG. 26.

### 1.4.1.6 DK365 (DK221-PEG12-NOTA) N

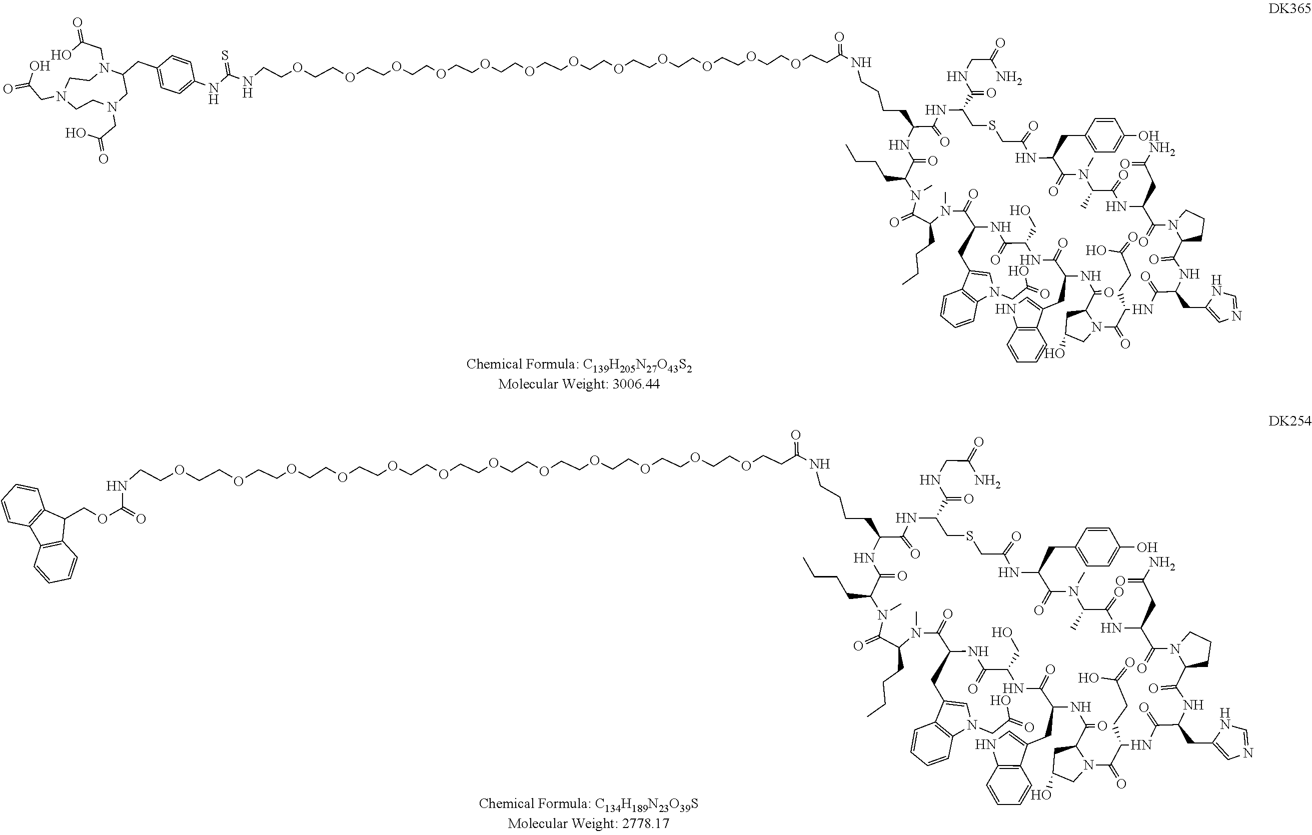

### 1.4.1.6.A Step-1: Synthesis of DK254

### 1.4.1.6.A.i Structure of DK254

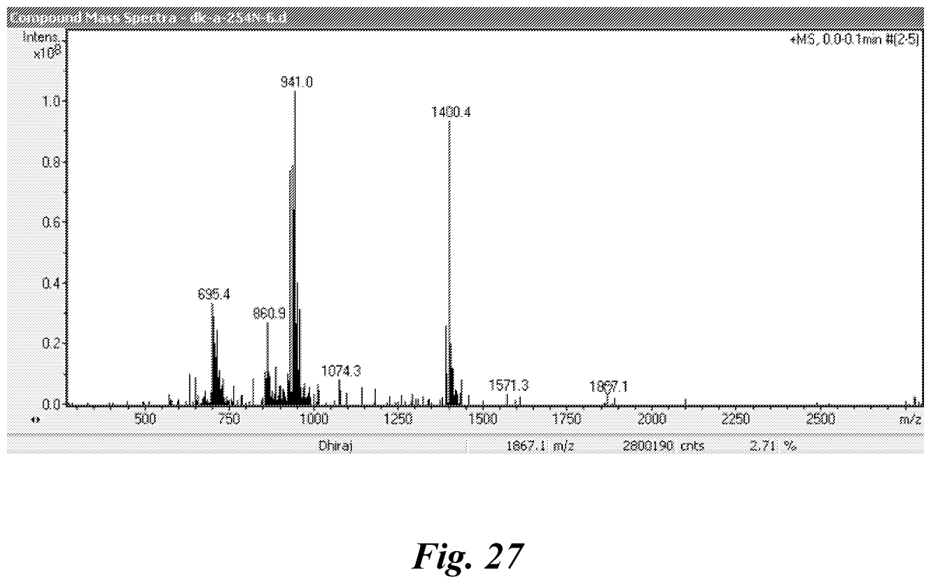

1.4.1.6.A.ii Procedure: To a stirred solution of DK221 (5.0 mg, 2.55 μmoles) in a 20 mL vial in Dimethylformamide (1.0 mL) was added Diisopropylethylamine (5.0 μL) followed by Fmoc-N-amido-dPE12-NHS ester (2.0 mg, 2.13 μmoles). The reaction mixture was stirred for 3-4 h at room temperature. The reaction mixture was purified on a reversed phase high performance liquid chromatography (RP-HPLC) system using a semi-preparative C-18 Luna column (5 mm, 10×250 mm Phenomenex, Torrance, Calif.). The HPLC conditions for purification were 20-60% acetonitrile (0.1% trifluoroacetic acid) and H₂O (0.1% trifluoroacetic acid) in 25 min at a flow rate of 5 mL/min. The desired DK254 was lyophilized to powder form in 53% yield which was characterized by ESI MS. Calculated [M+Na+2H]²⁺: 1400.5, Observed: 1400.4. The ESI MS of DK254 is shown in FIG. 27.

### 1.4.1.6B. Step-2: Synthesis of DK265

### 1.4.1.6.B. i Structure of DK265

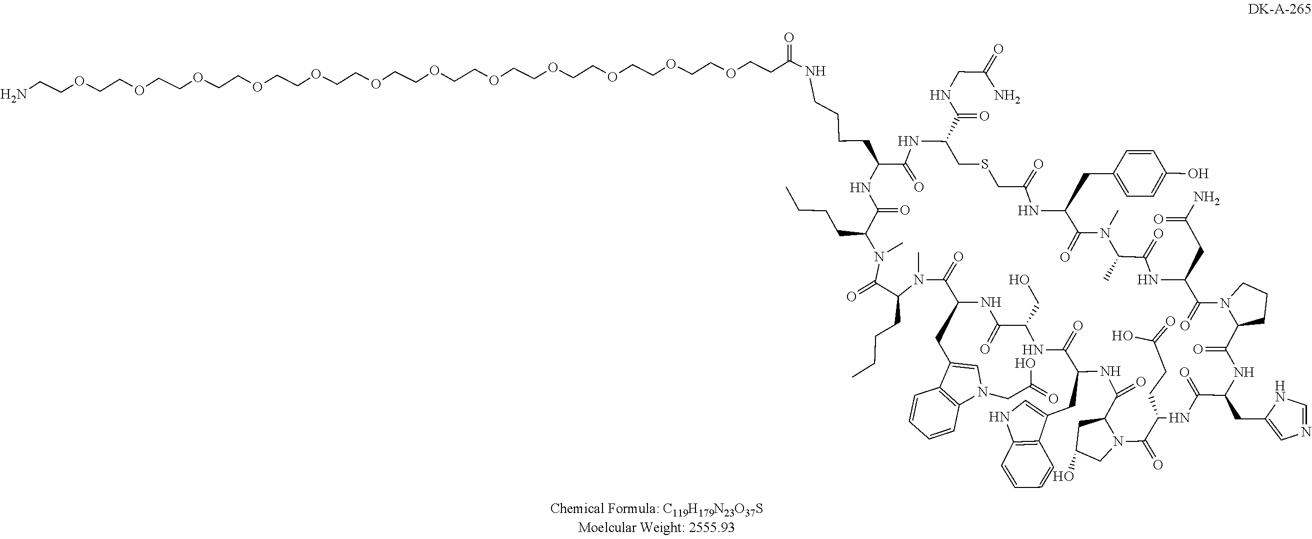 [SMILES: CCCC[C@H]1C(=O)N(C)[C@@H](CCCC)C(=O)N[C@@H](CCCCNC(=O)CCOCCOCCOCCOCCOCCOCCOCCOCCOCCOCCOCCOCCN)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](Cc2cnc[nH]2)C(=O)N[C@@H](CCC(=O)O)C(=O)N2C[C@H](O)C[C@H]2C(=O)N[C@@H](Cc2c[nH]c3ccccc23)C(=O)N[C@@H](CO)C(=O)N[C@@H](Cc2cn(CC(=O)O)c3ccccc23)C(=O)N1C]

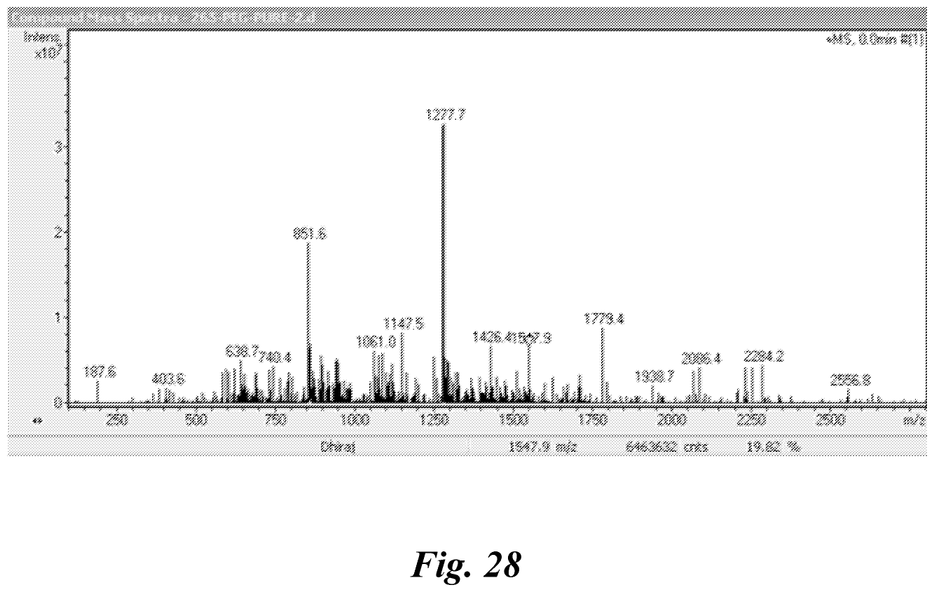

1.4.1.6B.ii Procedure: Stirred a solution of DK254 (5 mg) in a 20 mL vial in Dimethylformamide: Piperidine (1:1)(1.0 mL) for 2 h at room temperature. The crude reaction mixture was evaporated to dryness and used as for next step without further purification. The crude DK265 was obtained in quantitative which was characterized by ESI MS. Calculated [M+2H]²⁺: 1277.7, Observed: 1277.5. The ESI-MS of DK265 is shown in FIG. 28

1.4.1.6.C. Step-3: Synthesis of DK365

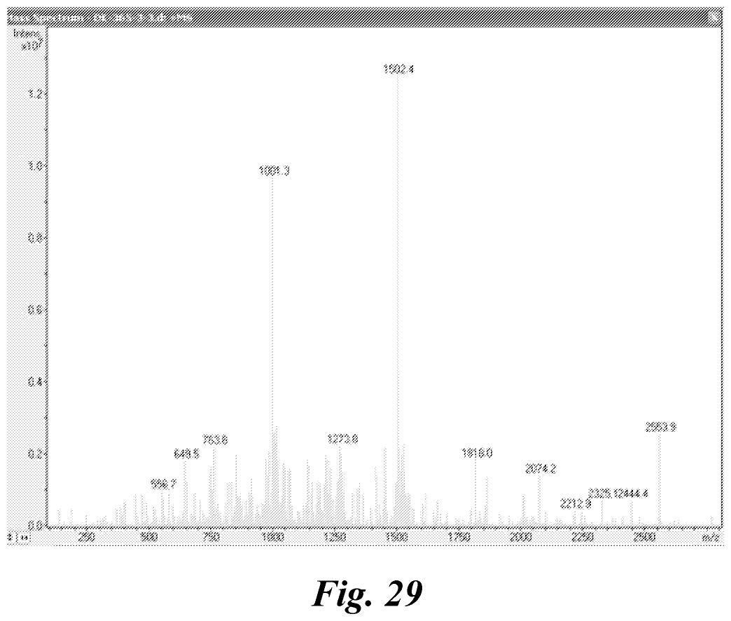

1.4.1.6.C.i. Procedure: To a stirred solution of DK265 (5.0 mg, 2.0 μmoles) in a 20 mL vial in Dimethylformamide (1.0 mL) was added Diisopropylethylamine (5.0 μL) followed by pSCN-Bn-NOTA (2.0 mg, 3.57 μmoles). The reaction mixture was stirred for 3-4 h at room temperature. The reaction mixture was purified on a reversed phase high performance liquid chromatography (RP-HPLC) system using a semi-preparative C-18 Luna column (5 mm, 10×250 mm Phenomenex, Torrance, Calif.). The HPLC conditions for purification were 20-60% acetonitrile (0.10% trifluoroacetic acid) and H₂O (0.1% trifluoroacetic acid) in 25 min at a flow rate of 5 m/min. The desired DK365 was lyophilized to powder form in 45% yield which was characterized by ESI MS. Calculated [M+2H]²⁺: 1502.2, Observed: 1502.4. The ESI-MS of DK365 is shown in FIG. 29.

### 1.4.1.7. DK360 (DK221-PEG12-NODA)

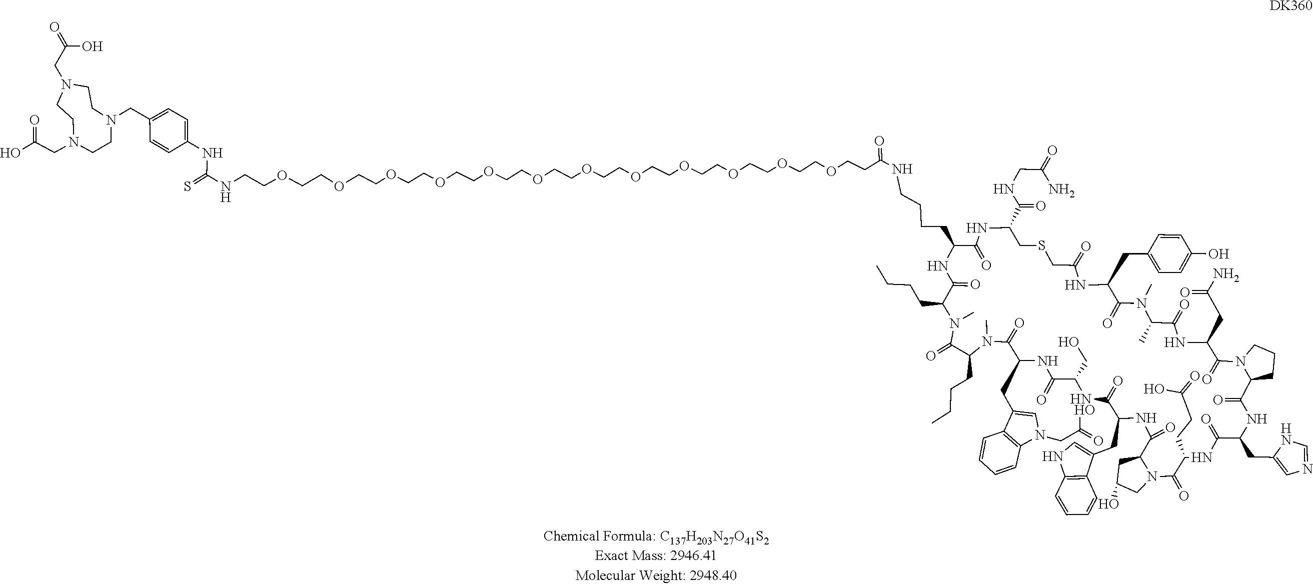

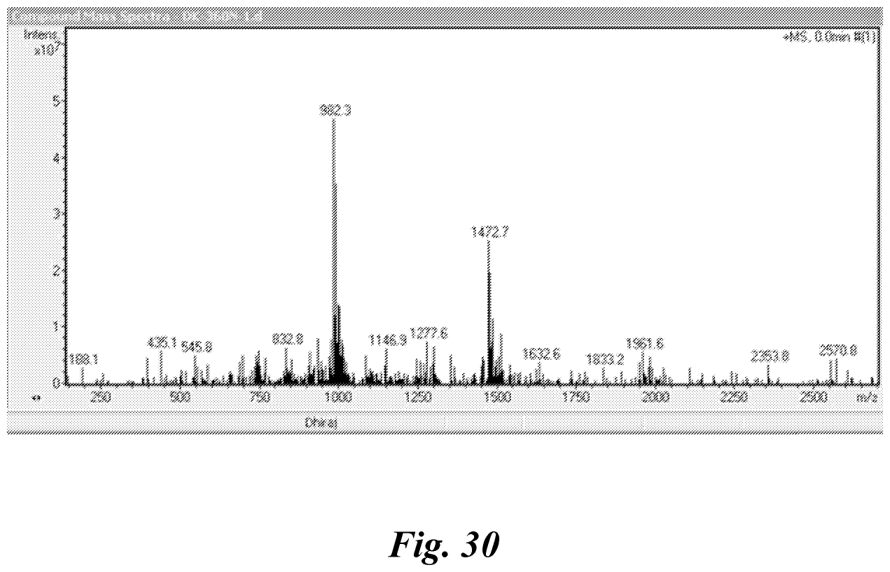

1.4.1.7.i. Procedure: To a stirred solution of DK265 (5.0 mg, 2.0 μmoles) in a 20 mL vial in Dimethylformamide (1.0 mL) was added Diisopropylethylamine (5.0 μL) followed by NCS-MP-NODA (1.2 mg, 3.0 μmoles). The reaction mixture was stirred for 2-3 h at room temperature. The reaction mixture was purified on a reversed phase high performance liquid chromatography (RP-HPLC) system using a semi-preparative C-18 Luna column (5 mm, 10×250 mm Phenomenex, Torrance, Calif.). The HPLC conditions for purification were 20-60% acetonitrile (0.1% trifluoroacetic acid) and H₂O (0.10% trifluoroacetic acid) in 25 min at a flow rate of 5 mL/min. The desired DK360 was lyophilized to powder form in 47% yield which was characterized by ESI MS. Calculated [M+2H]²⁺: 1473.0, Observed: 1472.7. The ESI-MS of DK360 is shown in FIG. 30.
1.4.1.8. DK388 (DK221 PEG4-alkyne)

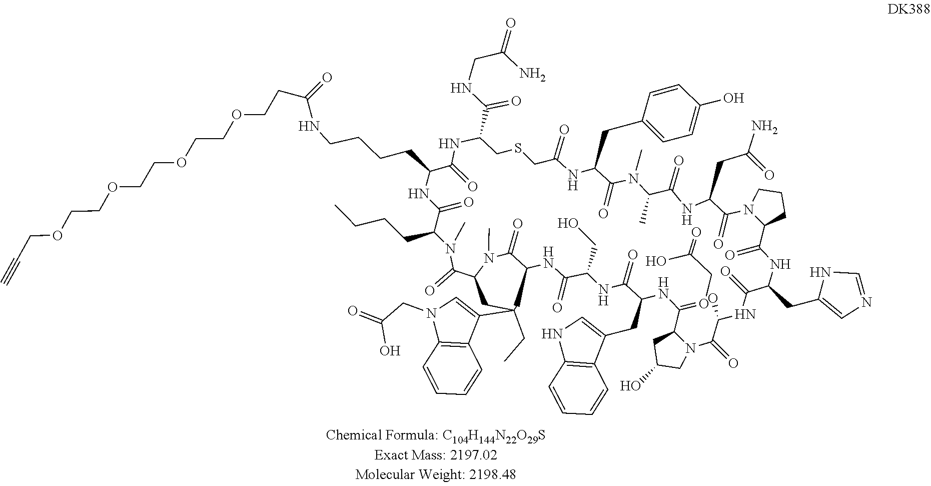

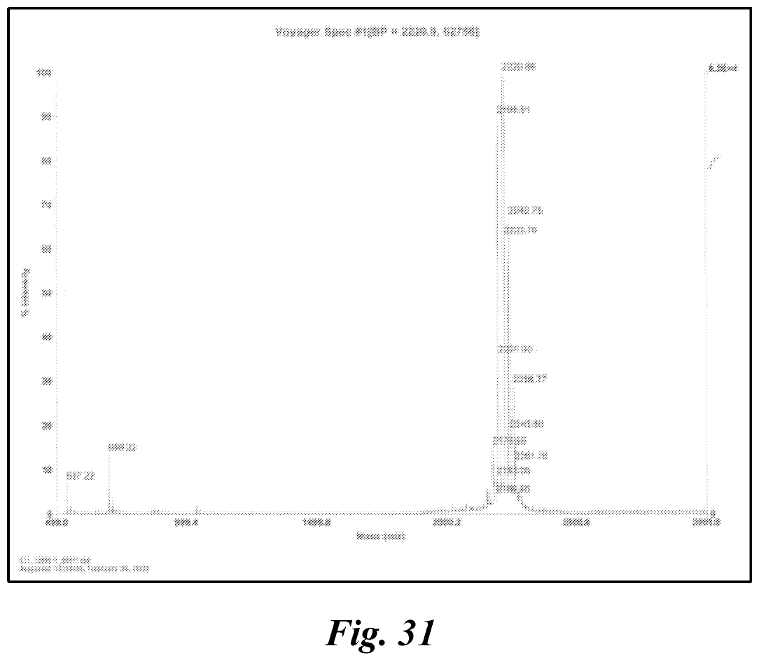

1.4.1.8.i. Procedure: To a stirred solution of DK265 (5.0 mg, 2.0 μmoles) in a 20 mL vial in Dimethylformamide (1.0 mL) was added Diisopropylethylamine (5.0 μL) followed by NCS-MP-NODA (1.2 mg, 3.0 μmoles). The reaction mixture was stirred for 2-3 h at room temperature. The reaction mixture was purified on a reversed phase high performance liquid chromatography (RP-HPLC) system using a semi-preparative C-18 Luna column (5 mm, 10×250 mm Phenomenex, Torrance, Calif.). The HPLC conditions for purification were 30-60% acetonitrile (0.1% formic acid) and H₂O (0.1% formic acid) in 25 min at a flow rate of 5 mL/min. The desired DK388 was lyophilized to powder form in 58% yield which was characterized by matrix assisted laser desorption/ionization time-of-flight mass spectrometry (MALDI-TOF MS). Calculated [M]⁺: 2198.48, Observed: 2198.91. The ESI-MS of DK388 is shown in FIG. 31.

### 1.4.1.9. Synthesis of [¹⁸F]DK221Py

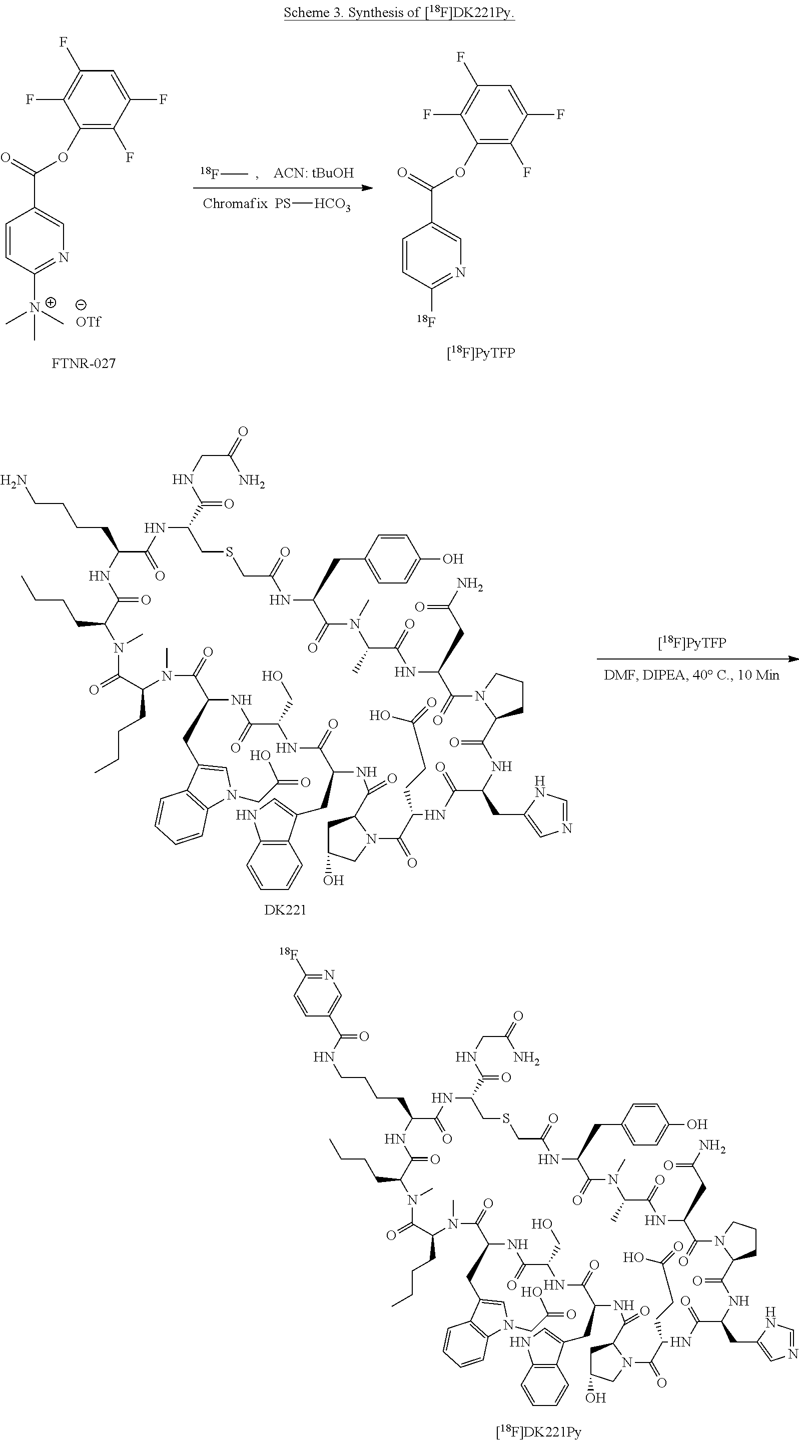

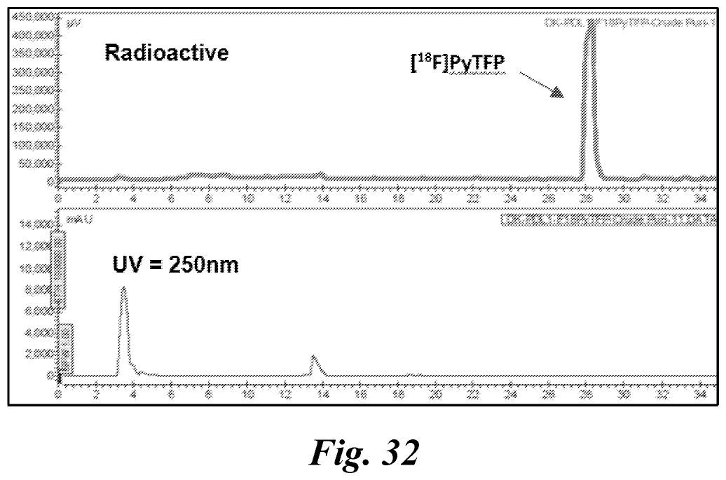

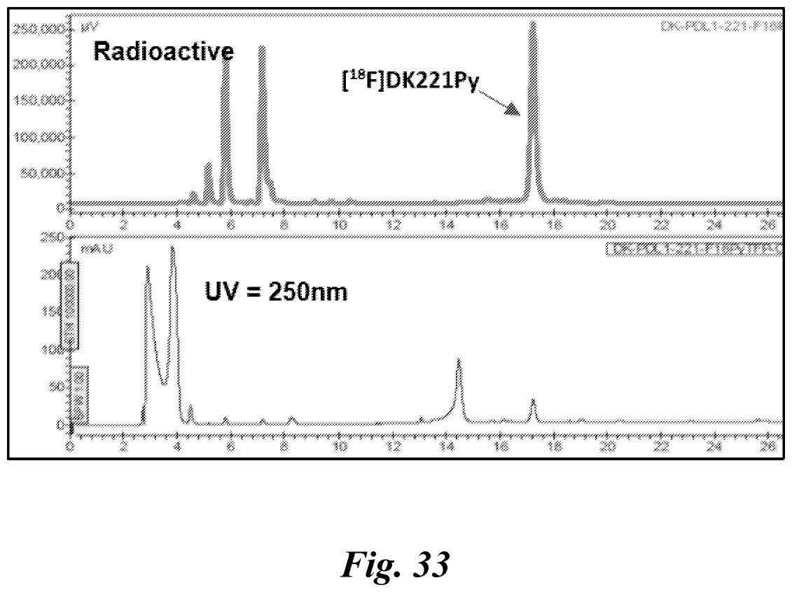

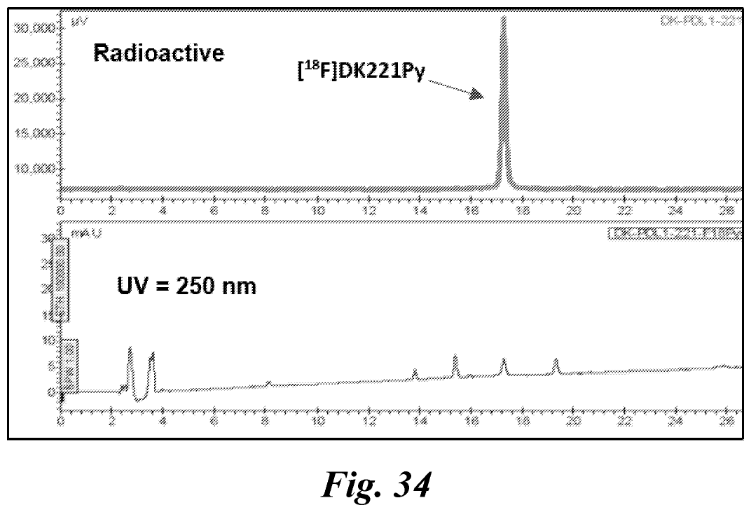

The RP-HPLC of crude [¹⁸F]PyTFP is shown in FIG. 32. The RP-HPLC of crude [¹⁸F]DK221Py is shown in FIG. 33. The RP-HPLC of pure [¹⁸F]DK221Py is shown in FIG. 34.

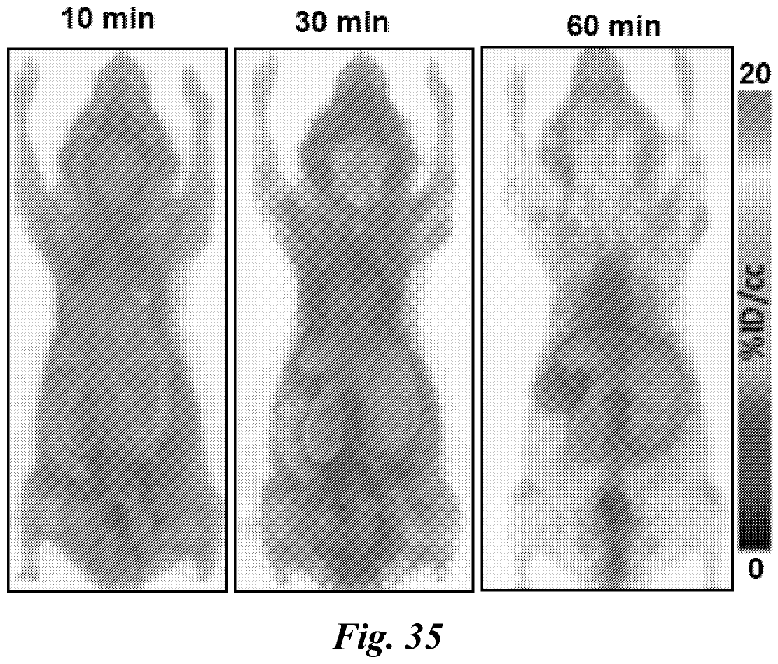

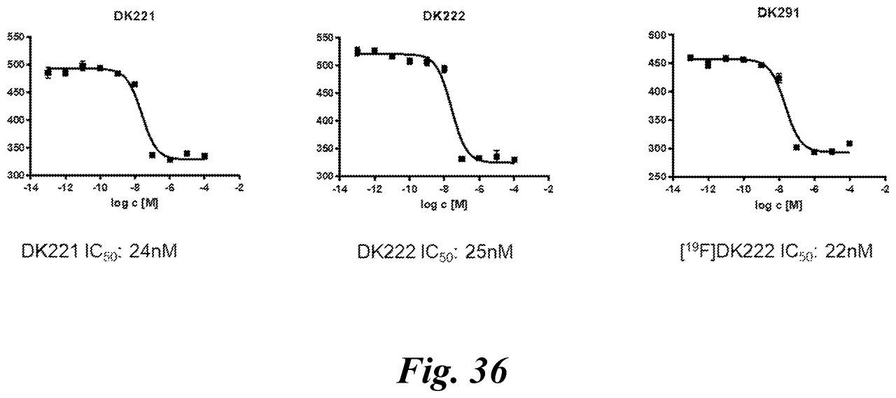

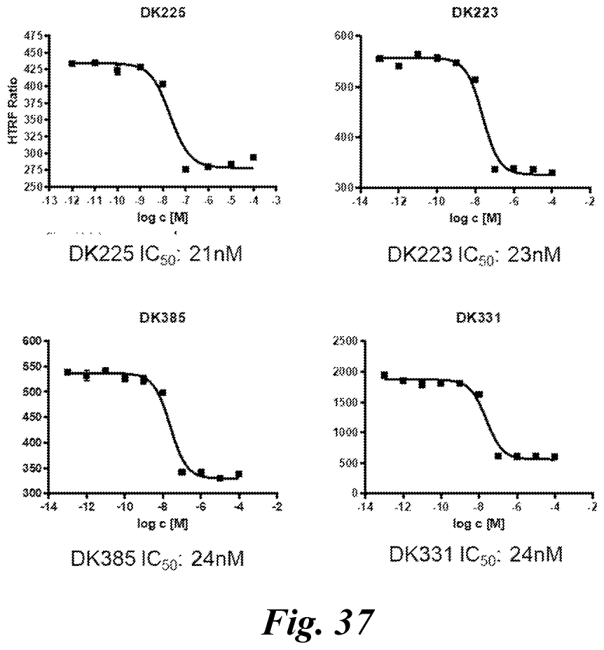

Further, the in vivo evaluation of [¹⁸F]DK221Py in hPD-L1/CHO xenografts is shown in FIG. 35.

### REFERENCES

All publications, patent applications, patents, and other references mentioned in the specification are indicative of the level of those skilled in the art to which the presently disclosed subject matter pertains. All publications, patent applications, patents, and other references (e.g., websites, databases, etc.) mentioned in the specification are herein incorporated by reference in their entirety to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
Ribas, A. and J. D. Wolchok, Cancer immunotherapy using checkpoint blockade. Science, 2018. 359(6382): p. 1350-1355.
Havel, J. J., D. Chowell, and T. A. Chan, The evolving landscape of biomarkers for checkpoint inhibitor immunotherapy. Nat Rev Cancer, 2019. 19(3): p. 133-150.
Garon, E. B., et al., Pembrolizumab for the treatment of non-small-cell lung cancer. N Engl J Med, 2015. 372(21): p. 2018-28.
Reck, M., et al., Pembrolizumab versus Chemotherapy for PD-L1-Positive Non-Small-Cell Lung Cancer. N Engl J Med, 2016. 375(19): p. 1823-1833.
Reck, M., et al., Updated Analysis of KEYNOTE-024: Pembrolizumab Versus Platinum-Based Chemotherapy for Advanced Non-Small-Cell Lung Cancer With PD-L1 Tumor Proportion Score of 50% or Greater. J Clin Oncol, 2019. 37(7): p. 537-546.
Herbst, R. S., et al., LBA79-Association between tissue TMB (tTMB) and clinical outcomes with pembrolizumab monotherapy (pembro) in PD-L1-positive advanced NSCLC in the KEYNOTE-010 and -042 trials. Annals of Oncology, 2019. 30: p. v916-v917.
Hellmann, M. D., et al., Nivolumab plus Ipilimumab in Lung Cancer with a High Tumor Mutational Burden. N Engl J Med, 2018. 378(22): p. 2093-2104.
Peters, S., et al., LBA4_PR—Nivolumab (NIVO)+low-dose ipilimumab (IPI) vs platinum-doublet chemotherapy (chemo) as first-line (1L) treatment (tx) for advanced non-small cell lung cancer (NSCLC): CheckMate 227 part I final analysis. Annals of Oncology, 2019. 30: p. v913-v914.
Spigel, D., et al., LBA78—IMpower110: Interim overall survival (OS) analysis of a phase III study of atezolizumab (atezo) vs platinum-based chemotherapy (chemo) as first-line (1L) treatment (tx) in PD-L1-selected NSCLC. Annals of Oncology, 2019. 30: p. v915.
Yarchoan, M., et al., PD-L1 expression and tumor mutational burden are independent biomarkers in most cancers. JCI Insight, 2019. 4(6).
Melosky, B., et al., Breaking the biomarker code: PD-L1 expression and checkpoint inhibition in advanced NSCLC. Cancer Treat Rev, 2018. 65: p. 65-77.
McLaughlin, J., et al., Quantitative Assessment of the Heterogeneity of PD-L1 Expression in Non-Small-Cell Lung Cancer. JAMA Oncol, 2016. 2(1): p. 46-54.
Bensch, F., et al., (89)Zr-atezolizumab imaging as a non-invasive approach to assess clinical response to PD-L1 blockade in cancer. Nat Med, 2018. 24(12): p. 1852-1858.
Niemeijer, A N., et al., Whole body PD-1 and PD-L1 positron emission tomography inpatients with non-small-cell lung cancer. Nat Commun, 2018. 9(1): p. 4664.
Kumar, D., et al., Peptide-based PET quantifies target engagement of PD-L1 therapeutics. J Clin Invest, 2019. 129(2): p. 616-630.
Chatterjee, S., et al., Rapid PD-L1 detection in tumors with PET using a highly specific peptide. Biochem Biophys Res Commun, 2017. 483(1): p. 258-263.
Miller, M. M., et al., Macrocyclic inhibitors of the PD1/PDL1 and CD80 (B7-1)/PD-L1 protein/protein interactions. 2016, Bristol-Myers Squibb Company, USA. p. 1093pp.
McBride, W. J., et al., Improved 18F labeling of peptides with a fluoride-aluminum-chelate complex. Bioconjug Chem, 2010. 21(7): p. 1331-40.
Kumar, K. and A. Ghosh, (18)F-AlF Labeled Peptide and Protein Conjugates as Positron Emission Tomography Imaging Pharmaceuticals. Bioconjug Chem, 2018. 29(4): p. 953-975.
Topalian, S. L., C. G. Drake, and D. M. Pardoll, Immune checkpoint blockade: a common denominator approach to cancer therapy. Cancer Cell, 2015. 27(4): p. 450-61.
Minn, A. J. and E. J. Wherry, Combination Cancer Therapies with Immune Checkpoint Blockade: Convergence on Interferon Signaling. Cell, 2016. 165(2): p. 272-5.
Huang, A. C., et al., A single dose of neoadjuvant PD-1 blockade predicts clinical outcomes in resectable melanoma. Nat Med, 2019. 25(3): p. 454-461.
Taube, J. M., et al., Colocalization of inflammatory response with B7-hl expression in human melanocytic lesions supports an adaptive resistance mechanism of immune escape. Sci Transl Med, 2012. 4(127): p. 127ra37.
Jia Xin Yu, et al., Trends in clinical development for PD-1/PD-L1 inhibitors. Nature Reviews Drug Discovery, 2019.
de Vries, E. G. E., et al., Integrating molecular nuclear imaging in clinical research to improve anticancer therapy. Nat Rev Clin Oncol, 2019. 16(4): p. 241-255.
Josefsson, A., et al., Imaging, biodistribution, and dosimetry of radionuclide-labeled PD-L1 antibody in an immunocompetent mouse model of breast cancer. Cancer Res, 2015.
Maute, R. L., et al., Engineering high-affinity PD-1 variants for optimized immunotherapy and immuno-PET imaging. Proc Natl Acad Sci USA, 2015. 112(47): p. E6506-14.
Chatterjee, S., et al., A humanized antibody for imaging immune checkpoint ligand PD-L1 expression in tumors. Oncotarget, 2016. 7(9): p. 10215-27.
Truillet, C., et al., Imaging PD-L1 expression with immunoPET. Bioconjug Chem, 2017.
De Silva, R. A., et al., Peptide-Based (68)Ga-PET Radiotracer for Imaging PD-L1 Expression in Cancer. Mol Pharm, 2018. 15(9): p. 3946-3952.
Jagoda, E. M., et al., Immuno-PET Imaging of the Programmed Cell Death-1 Ligand (PD-L1) Using a Zirconium-89 Labeled Therapeutic Antibody, Avelumab. Mol Imaging, 2019. 18: p. 1536012119829986.
Vento, J., et al., PD-L1 detection using (89)Zr-atezolizumab immuno-PET in renal cell carcinoma tumorgrafts from a patient with favorable nivolumab response. J Immunother Cancer, 2019. 7(1): p. 144.
Wissler, H. L., et al., Site-Specific Immuno-PET Tracer to Image PD-L1. Mol Pharm, 2019. 16(5): p. 2028-2036.
Hettich, M., et al., High-Resolution PET Imaging with Therapeutic Antibody-based PD-1/PD-L1 Checkpoint Tracers Theranostics, 2016. 6(10): p. 1629-1640.
Donnelly, D. J., et al., Synthesis and Biologic Evaluation of a Novel (18)F-Labeled Adnectin as a PET Radioligand for Imaging PD-L1 Expression. J Nucl Med, 2018. 59(3): p. 529-535.
Lesniak, W. G., et al., Development of [(18)F]FPy-WL12 as a PD-L1 Specific PET Imaging Peptide. Mol Imaging, 2019. 18: p. 1536012119852189.
Heskamp, S., et al., PD-L1 microSPECT/CT Imaging for Longitudinal Monitoring of PD-L1 Expression in Syngeneic and Humanized Mouse Models for Cancer. Cancer Immunol Res, 2019. 7(1): p. 150-161.
Ehlerding, E. B., et al., Noninvasive Imaging and Quantification of Radiotherapy-Induced PD-L1 Upregulation with (89)Zr-Df-Atezolizumab. Bioconjug Chem, 2019. 30(5): p. 1434-1441.
Kikuchi, M., et al., Preclinical immunoPET/CT imaging using Zr-89-labeled anti-PD-L1 monoclonal antibody for assessing radiation-induced PD-L1 upregulation in head and neck cancer and melanoma. Oncoimmunology, 2017. 6(7): p. e1329071.
Chatterjee, S., W. G. Lesniak, and S. Nimmagadda, Noninvasive Imaging of Immune Checkpoint Ligand PD-L1 in Tumors and Metastases for Guiding Immunotherapy. Mol Imaging, 2017. 16: p. 1536012117718459.
Broos, K., et al., Non-invasive assessment of murine PD-L1 levels in syngeneic tumor models by nuclear imaging with nanobody tracers. Oncotarget, 2017. 8(26): p. 41932-41946.
Josefsson, A., et al., Imaging, Biodistribution, and Dosimetry of Radionuclide-Labeled PD-L1 Antibody in an Immunocompetent Mouse Model of Breast Cancer. Cancer Res, 2016. 76(2): p. 472-9.
Nataraj an, A., et al., Novel Radiotracer for ImmunoPET Imaging of PD-1 Checkpoint Expression on Tumor Infiltrating Lymphocytes. Bioconjug Chem, 2015.
Heskamp, S., et al., Noninvasive Imaging of Tumor PD-L1 Expression Using Radiolabeled Anti-PD-L1 Antibodies. Cancer Res, 2015. 75(14): p. 2928-36.
Szabo, Z., et al., Initial evaluation of[(18)F]DCFPyL for prostate-specific membrane antigen (PSMA)-targeted PET imaging of prostate cancer. Mol Imaging Biol, 2015. 17(4): p. 565-74.
Herrmann, K., et al., Biodistribution and radiation dosimetry for the chemokine receptor CXCR4-targeting probe 68Ga-pentixafor. J Nucl Med, 2015. 56(3): p. 410-6.
Tan, S., et al., Distinct PD-L1 binding characteristics of therapeutic monoclonal antibody durvalumab. Protein Cell, 2017.
Agoram, B. M., S. W. Martin, and P. H. van der Graaf, The role of mechanism-based pharmacokinetic-pharmacodynamic (PK-PD) modelling in translational research of biologics. Drug Discov Today, 2007. 12(23-24): p. 1018-24.
Agoram, B. M., Use ofpharmacokinetic/pharmacodynamic modelling for starting dose selection in first-in-human trials of high-risk biologics. Br J Clin Pharmacol, 2009. 67(2): p. 153-60.
Giesen, D., et al., 8089Zr-labeled anti-PD-L1 CX-072 PET imaging in human xenograft and syngeneic tumors. Ann Oncol, 2019. 30(Supplement_1).
Lan, Y., et al., Enhanced preclinical antitumor activity of M7824, a bifunctional fusion protein simultaneously targeting PD-L1 and TGF-beta. Sci Transl Med, 2018. 10(424).
Lesniak, W. G., et al., PD-L1 Detection in Tumors Using [(64)CuJAtezolizumab with PET. Bioconjug Chem, 2016. 27(9): p. 2103-10.
Gniadek, T. J., et al., Heterogeneous expression of PD-L1 in pulmonary squamous cell carcinoma and adenocarcinoma: implications for assessment by small biopsy. Mod Pathol, 2017. 30(4): p. 530-538.
WO/2017/201 111 for PET-IMAGING IMMUNOMODULATORS, to Donnelly et al., published Nov. 23, 2017.
WO2018119313 for TUMOR AND IMMUNE CELL IMAGING BASED ON PD-L1 EXPRESSION, to Nimmagadda et al., published Jun. 28, 2018.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. An imaging agent comprising a compound of formula (I): 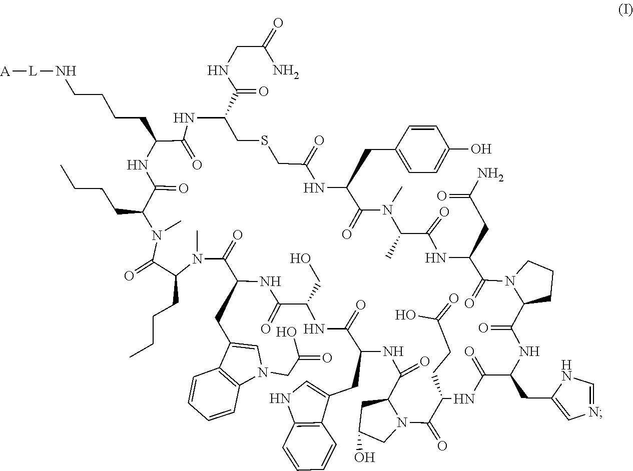
wherein:
L is a linker, which can be present or absent, and when present has the following general formula: 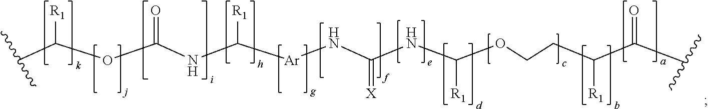
wherein:
X is S or O;
a, e, f, g, i, and j are each independently an integer selected the group consisting of 0 and 1;
b, d, h, and k are each independently an integer selected from the group consisting of 0, 1, 2, 3, 4, 5, 6, 7, and 8;
c is an integer having a range from 0 to 40;
each R₁ is H or —COOR₂, wherein R₂ is H or C₁-C₄ alkyl; Ar is substituted or unsubstituted aryl or heteroaryl; and
A is a reporting moiety selected from the group consisting of a chelating agent, a radiolabeled substrate, a fluorescent dye, a photoacoustic reporting molecule, and a Raman-active reporting molecule or an end group selected from the group consisting of —NR₃R₄ or C≡N, wherein R₃ and R₄ are each independently selected from the group consisting of H and C₁-C₄ alkyl.

2. The imaging agent of claim 1, wherein the linker is selected from the group consisting of: 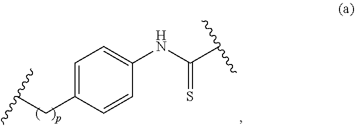
wherein p is an integer selected from 0, 1, 2, 3, and 4; 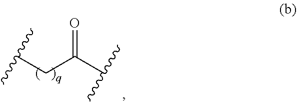
wherein q is an integer selected from the group consisting of 0, 1, 2, 3, 4, 5, 6, 7, and 8; 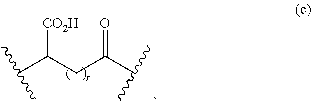
wherein r is an integer selected from the group consisting of 0, 1, 2, 3, 4, 5, 6, 7, and 8; 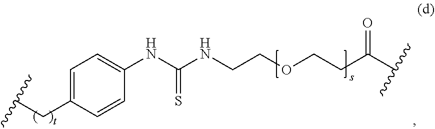
wherein s is an integer having a range from 1 to 40 and t is an integer selected from 0 or 1; 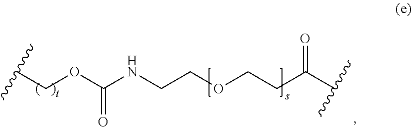
wherein s is an integer having a range from 1 to 40 and t is an integer selected from 0 or 1; and 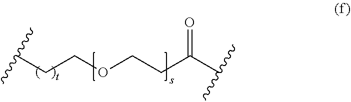
wherein s is an integer having a range from 1 to 40 and t is an integer selected from 0 or 1.

3. The imaging agent of claim 1, wherein the reporting moiety is a chelating agent and the chelating agent is selected from the group consisting of DOTAGA (1,4,7,10-tetraazacyclododececane, 1-(glutaric acid)-4,7,10-triacetic acid), DOTA (1,4,7,10-tetraazacyclododecane-1,4,7,10-tetraacetic acid), DOTA-tris(t-butyl)ester, DOTAGA-(t-butyl)₄, DOTA-di(t-butyl)ester, DOTASA (1,4,7,10-tetraazacyclododecane-1-(2-succinic acid)-4,7,10-triacetic acid), CB-DO2A (10-bis(carboxymethyl)-1,4,7,10-tetraazabicyclo[5.5.2]tetradecane), DEPA (7-[2-(Bis-carboxymethylamino)-ethyl]-4,10-bis-carboxymethyl-1,4,7,10-tetraaza-cyclododec-1-yl-acetic acid)), 3p-C-DEPA (2-[(carboxymethyl)][5-(4-nitrophenyl-1-[4,7,10-tris(carboxymethyl)-1,4,7,10-tetraazacyclododecan-1-yl]pentan-2-yl)amino]acetic acid)), TCMC (2-(4-isothiocyanotobenzyl)-1,4,7,10-tetraaza-1,4,7,10-tetra-(2-carbamonyl methyl)-cyclododecane), oxo-DO3A (1-oxa-4,7,10-triazacyclododecane-5-S-(4-isothiocyanatobenzyl)-4,7,10-triacetic acid), DO3A-(t-butyl), DO3AM (2,2′,2″-(1,4,7,10-tetraazacyclododecane-1,4,7-triyl)triacetamide), p-NH₂-Bn-Oxo-DO3A (1-Oxa-4,7,10-tetraazacyclododecane-5-S-(4-aminobenzyl)-4,7,10-triacetic acid), TE2A ((1,8-N,N-bis-(carboxymethyl)-1,4,8,11-tetraazacyclotetradecane), MM-TE2A, DM-TE2A, CB-TE2A (4,11-bis(carboxymethyl)-1,4,8,11-tetraazabicyclo[6.6.2]hexadecane), CB-TE1A1P (4,8,11-tetraazacyclotetradecane-1-(methanephosphonic acid)-8-(methanecarboxylic acid), CB-TE2P (1,4,8,11-tetraazacyclotetradecane-1,8-bis(methanephosphonic acid), TETA (1,4,8,11-tetraazacyclotetradecane-1,4,8,11-tetraacetic acid), NOTA (1,4,7-triazacyclononane-N,N′,N″-triacetic acid), NOTA(t-butyl)₂, NO2A (1,4,7-Triazacyclononane-1,4-bis(acetic acid)-7-(acetamide), NODA (1,4,7-triazacyclononane-1,4-diacetate); NODAGA (1,4,7-triazacyclononane, 1-glutaric acid-4,7-acetic acid), NODAGA(t-butyl)₃, NOTAGA (1,4,7-triazonane-1,4-diyl)diacetic acid), DFO (Desferoxamine), DTPA (2-[Bis[2-[bis(carboxymethyl)amino]ethyl]amino]acetic acid), DTPA-tetra(t-butyl)ester (diethylenetriamine-N,N,N′,N″-tetra-tert-butyl acetate-N′-acetic acid), NETA ([4-[2-(bis-carboxymethylamino)-ethyl]-7-carboxymethl-[1,4,7]triazonan-1-yl}-acetic acid), TACN-TM (N,N′,N″, tris(2-mercaptoethyl)-1,4,7-triazacyclononane), Diamsar (1,8-Diamino-3,6,10,13,16,19-hexaazabicyclo(6,6,6)eicosane, 3,6,10,13,16,19-Hexaazabicyclo[6.6.6]eicosane-1,8-diamine), Sarar (1-N-(4-aminobenzyl)-3,6,10,13,16,19-hexaazabicyclo[6.6.6] eicosane-1,8-diamine), AmBaSar (4-((8-amino-3,6,10,13,16,19-hexaazabicyclo [6.6.6] icosane-1-ylamino) methyl) benzoic acid), BaBaSar, tris(hydroxypyridinone) (THP), THP(benzyl)₃, NOPO (3-(((4,7-bis((hydroxy(hydroxymethyl)phosphoryl)-methyl)-1,4,7-triazonan-1-yl)methyl)(hydroxy)phosphoryl)propanoic acid), TRAP (3,3′,3″-(((1,4,7-triazonane-1,4,7-triyl)tris(methylene))tris(hydroxyphosphoryl))-tripropanoic acid), p-NH₂—Bn-PCTA (3,6,9,15-Tetraazabicyclo[9.3.1] pentadeca-1(15),11,13-triene-4-S-(4-aminobenzyl)-3,6,9-triacetic acid), and biotin (5-[(3aS,4S,6aR)-2-oxohexahydro-TH-thieno[3,4-d]imidazol-4-yl]pentanoic acid).

4. The imaging agent of claim 3, wherein the chelating agent is selected from the group consisting of:

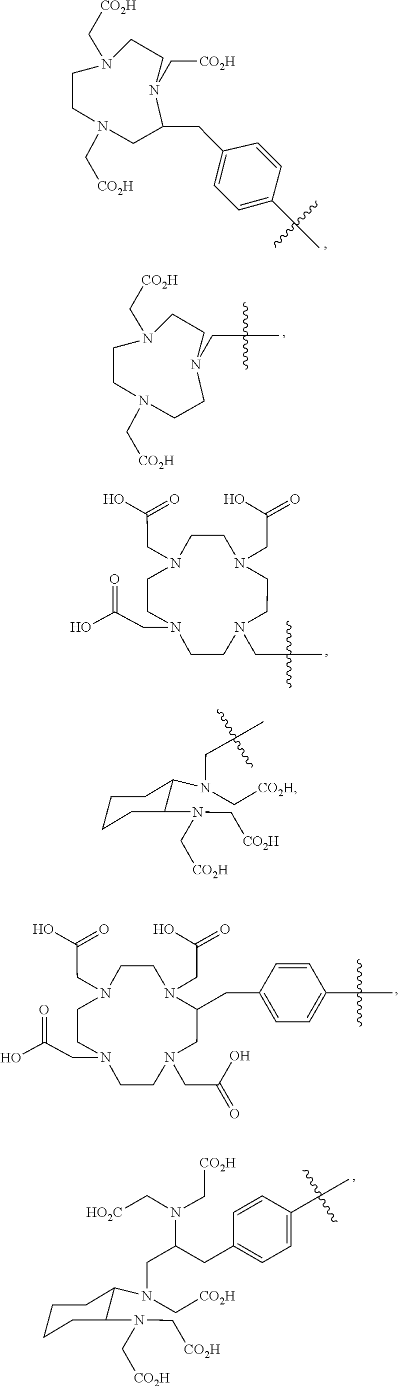

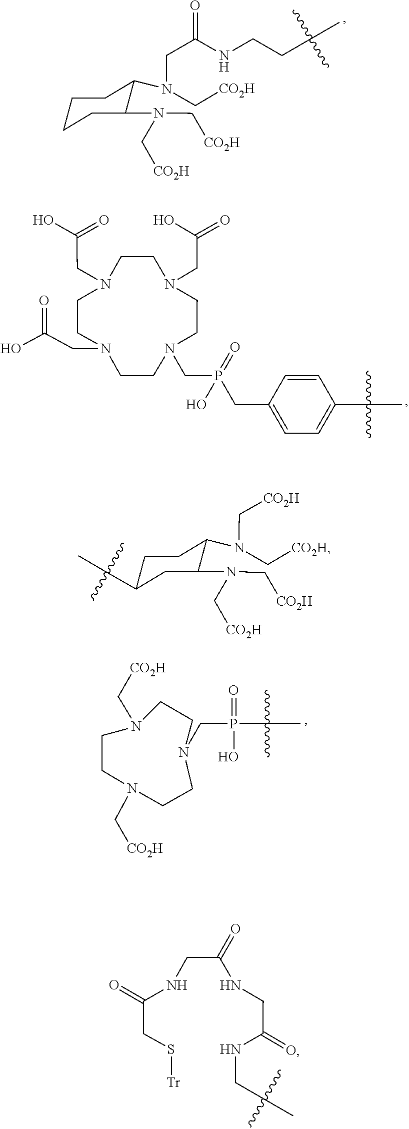

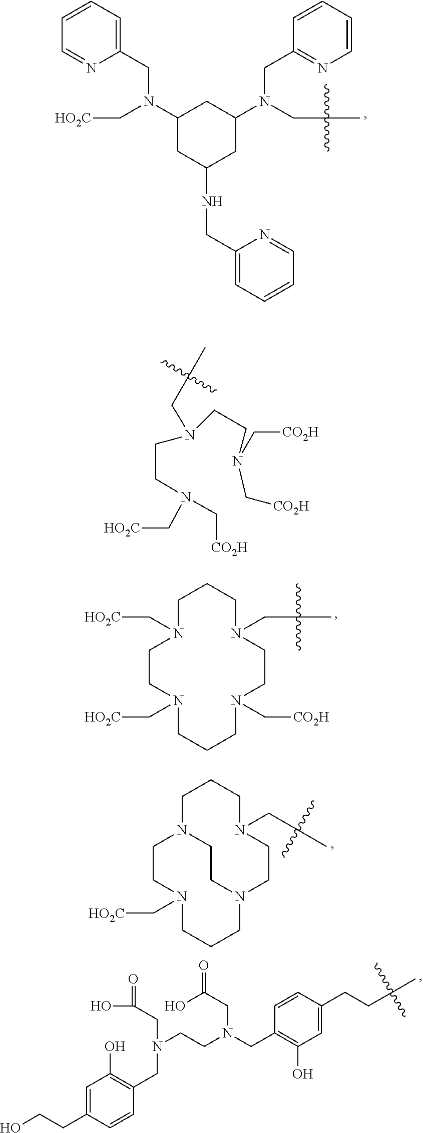

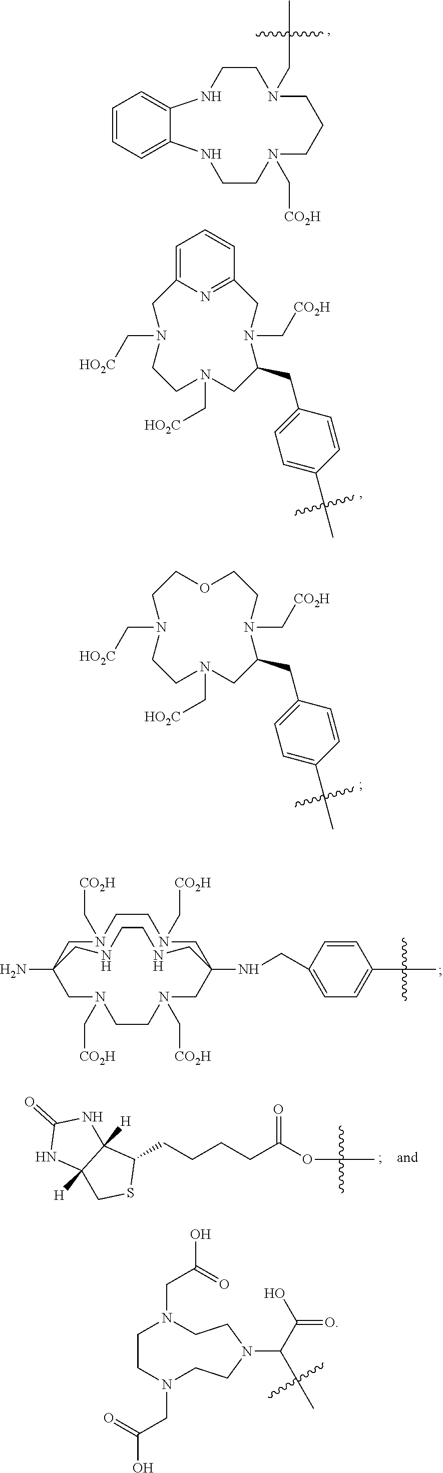 [SMILES: *CN1CCCN(CC(=O)O)CCNc2ccccc2NCC1 ; *c1ccc(C[C@H]2CN(CC(=O)O)CCN(CC(=O)O)Cc3cccc(n3)CN2CC(=O)O)cc1 ; *c1ccc(C[C@H]2CN(CC(=O)O)CCN(CC(=O)O)CCOCCN2CC(=O)O)cc1 ; *c1ccc(CNC23CNCCNCC(N)(CN(CC(=O)O)CCN(CC(=O)O)C2)CN(CC(=O)O)CCN(CC(=O)O)C3)cc1 ; *OC(=O)CCCC[C@@H]1SC[C@@H]2NC(=O)N[C@@H]21 ; *C(C(=O)O)N1CCN(CC(=O)O)CCN(CC(=O)O)CC1]

5. The imaging agent of claim 3, wherein the chelating agent is selected from the group consisting of DOTA (1,4,7,10-tetraazacyclododecane-1,4,7,10-tetraacetic acid), NOTA (1,4,7-triazacyclononane-N,N′,N″-triacetic acid), NODA (1,4,7-triazacyclononane-1,4-diacetate); NODAGA (1,4,7-triazacyclononane, 1-glutaric acid-4,7-acetic acid), and biotin (5-[(3aS,4S,6aR)-2-oxohexahydro-1H-thieno[3,4-d]imidazol-4-yl]pentanoic acid).

6. The imaging agent of claim 1, wherein the reporting moiety is a chelating agent and the chelating agent further comprises a radiometal selected from the group consisting of ^{94m}Tc, ^{99m}Tc, ¹¹¹In, ⁶⁷Ga, ⁶⁸Ga, ⁸⁶Y ⁹⁰Y, ¹⁷⁷Lu, ¹⁸⁶Re, ¹⁸⁸Re, ⁶⁰Cu, ⁶¹Cu, ⁶²Cu, ⁶⁴Cu, ⁶⁷Cu, ⁵⁵Co, ⁵⁷Co, ⁴⁴Sc, ⁴⁷Sc, ²²⁵Ac, ²¹³Bi, ²¹²Bi, ²¹²Pb, ¹⁵³Sm, ¹⁶⁶Ho, ¹⁵²Gd, ⁸²Rb, ⁸⁹Zr, ¹⁶⁶Dy, and Al¹⁸F.

7. The imaging agent of claim 1, wherein the reporting moiety is a radiolabeled substrate and the radiolabeled substrate comprises a radioisotope selected from the group consisting of ¹¹C, ¹³N, ¹⁵O, ¹²³I, ¹²⁴I, ¹²⁵I, ¹²⁶I, ¹³¹I, ⁷⁵Br, ⁷⁶Br, ⁷⁷Br, ⁸⁰Br, ^{80m}Br, ⁸²Br, ⁸³Br, ¹⁹F, ¹⁸F, and ²¹¹At.

8. The imaging agent of claim 7, wherein the radiolabeled substrate comprises an ¹⁸F-labeled substrate or an ¹⁸F-labeled substrate.

9. The imaging agent of claim 8, wherein the ¹⁹F-labeled substrate or the ¹⁸F-labeled substrate is selected from the group consisting of 2-fluoro-PABA, 3-fluoro-PABA, 2-fluoro-mannitol, and N-succinimidyl-4-fluorobenzoate, and 2-pyridyl.

10. The imaging agent of claim 1, wherein the reporting moiety is a fluorescent dye and the fluorescent dye is selected from the group consisting of: carbocyanine, indocarbocyanine, oxacarbocyanine, thuicarbocyanine, merocyanine, polymethine, coumarine, aminomethylcoumarin acetate (AMCA), rhodamine, tetramethylrhodamine (TRITC), xanthene, fluorescein, FITC, a boron-dipyrromethane (BODIPY) dye, Cy3, Cy5, Cy5.5, Cy7, VivoTag-680, VivoTag-S680, VivoTag-S750, AlexaFluor350, AlexaFluor405, AlexaFluor488, AlexaFluor546, AlexaFluor555, AlexaFluor594, AlexaFluor633, AlexaFluor647, AlexaFluor660, AlexaFluor680, AlexaFluor700, AlexaFluor750, AlexaFluor790, Dy677, Dy676, Dy682, Dy752, Dy780, DyLight350, DyLight405, DyLight488, DyLight547, DyLight550, DyLight594, DyLight633, DyLight647, DyLight650, DyLight680, DyLight755, DyLight800, HiLyte Fluor 647, HiLyte Fluor 680, HiLyte Fluor 750, IR Dye 800, IRDye 800CW, IRDye 800RS, IRDye 700DX, ADS780WS, ADS830WS, ADS832WS, Cascade Blue, and Texas Red.

11. The imaging agent of claim 1, wherein the reporting moiety is a photoacoustic reporting molecule and the photoacoustic reporting molecule is selected from the group consisting of a dye or a nanoparticle.

12. The imaging agent of claim 11, wherein the dye comprises a fluorescent dye.

13. The imaging agent of claim 12, wherein the fluorescent dye is selected from the group consisting of indocyanine-green (ICG), Alexa Fluor 750, Evans Blue, BHQ3, QXL680, IRDye880CW, MMPSense 680, Methylene Blue, PPCy-C8, and Cypate-C18.

14. The imaging agent of claim 11, wherein the nanoparticle is selected from the group consisting of a plasmonic nanoparticle, a quantum dot, a nanodiamond, a polypyrrole nanoparticle, a copper sulfide nanoparticle, a graphene nanosheet, an iron oxide-gold core-shell nanoparticle, a Gd₂O₃ nanoparticle, a single-walled carbon nanotube, a dye-loaded perfluorocarbon nanoparticle, and a superparamagnetic iron oxide nanoparticle.

15. The imaging agent of claim 1, wherein the reporting moiety is a Raman-active reporting molecule and the Raman-active reporting molecule is selected from the group consisting of a single-walled carbon nanotube (SWNT) and a surface-enhanced Raman scattering (SERS) agent.

16. The imaging agent of claim 15, wherein the SERS agent comprises a metal nanoparticle labeled with a Raman-active reporter molecule.

17. The imaging agent of claim 16, wherein the Raman-active reporter molecule comprises a fluorescent dye.

18. The imaging agent of claim 17, wherein the fluorescent dye is selected from the group consisting of Cy3, Cy5, rhodamine, and a chalcogenopyrylium dye.

19. The imaging agent of claim 1, wherein the imaging agent is selected from the group consisting of:

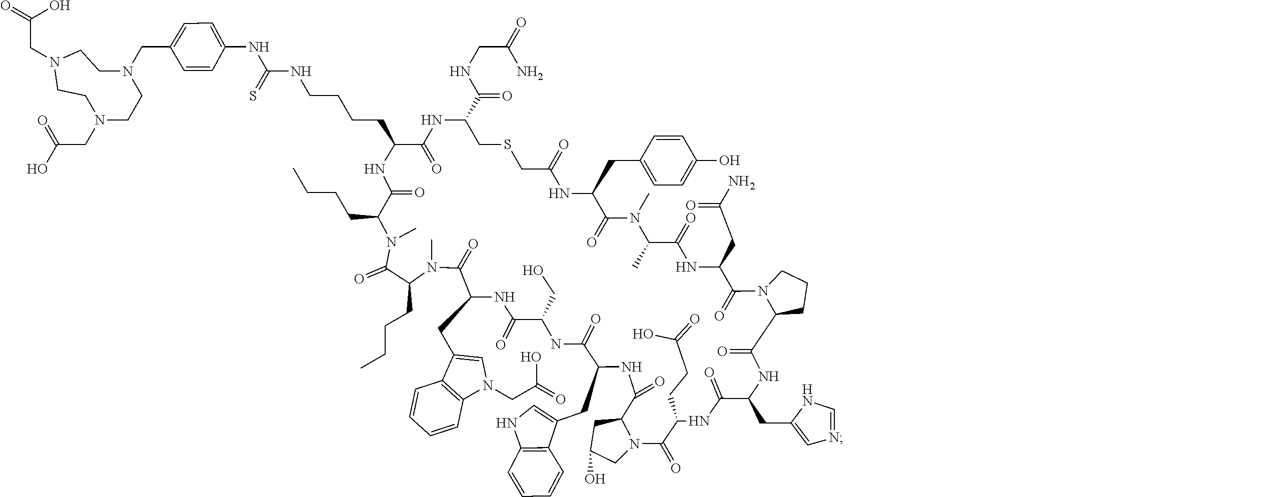

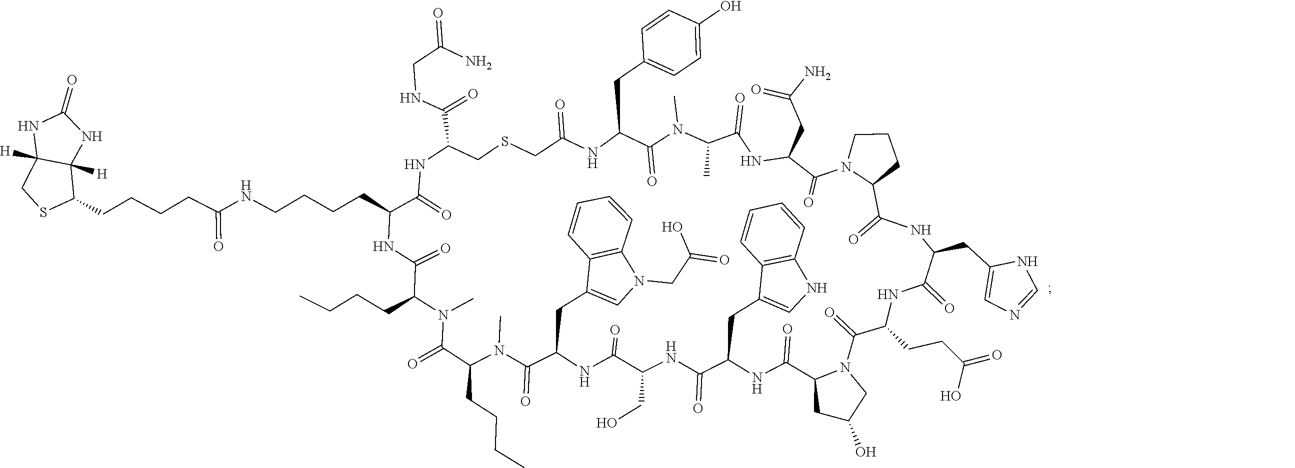 [SMILES: CCCC[C@H]1C(=O)N(C)[C@@H](CCCC)C(=O)N[C@@H](CCCCNC(=O)CCCC[C@@H]2SC[C@@H]3NC(=O)N[C@@H]32)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](Cc2cnc[nH]2)C(=O)N[C@H](CCC(=O)O)C(=O)N2C[C@H](O)C[C@H]2C(=O)N[C@H](Cc2c[nH]c3ccccc23)C(=O)N[C@H](CO)C(=O)N[C@H](Cc2cn(CC(=O)O)c3ccccc23)C(=O)N1C]

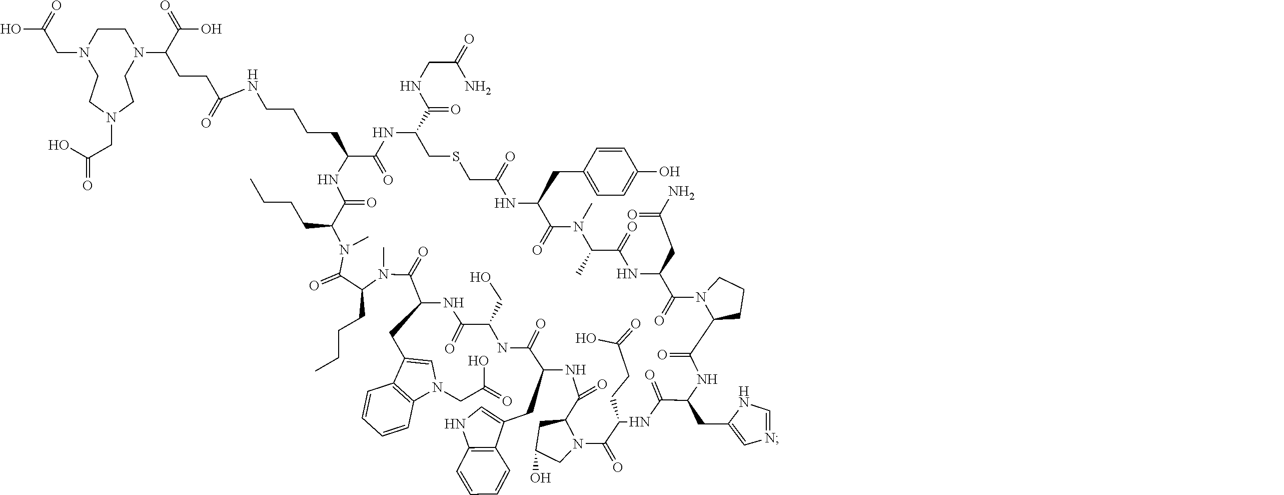

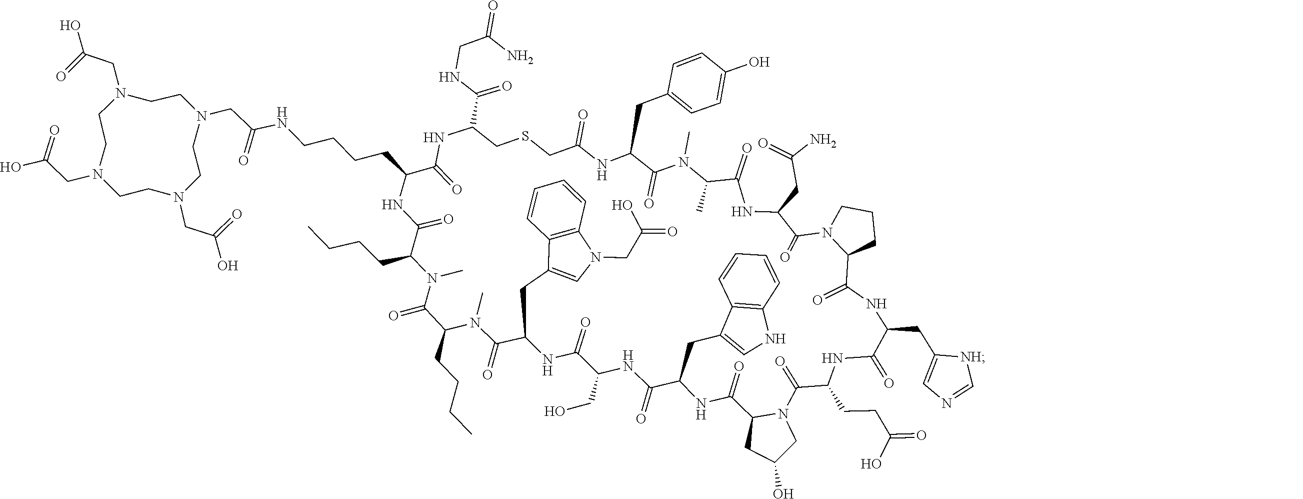

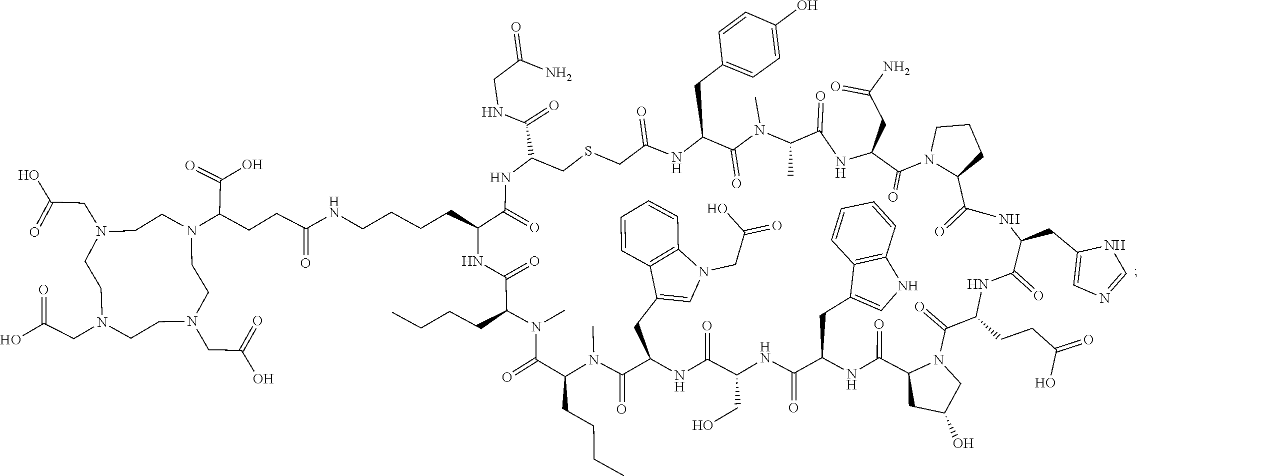

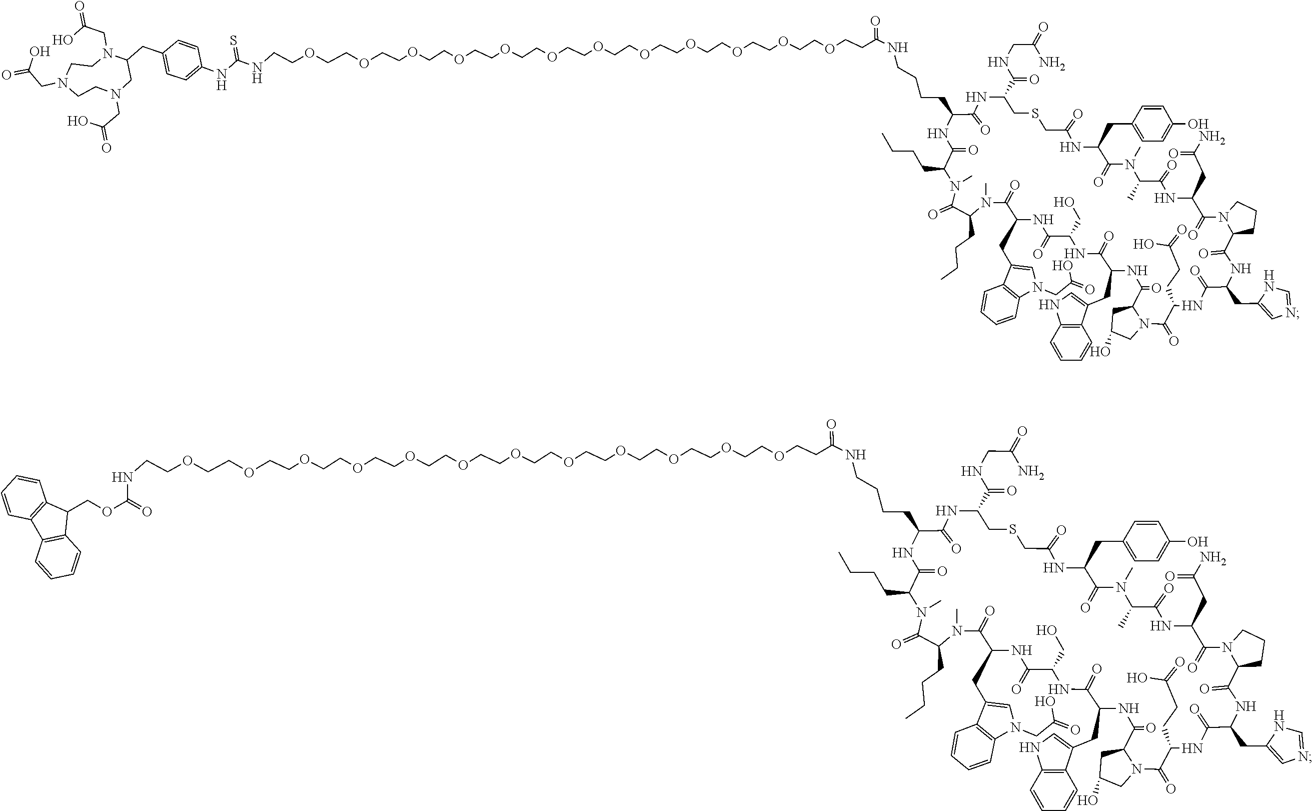

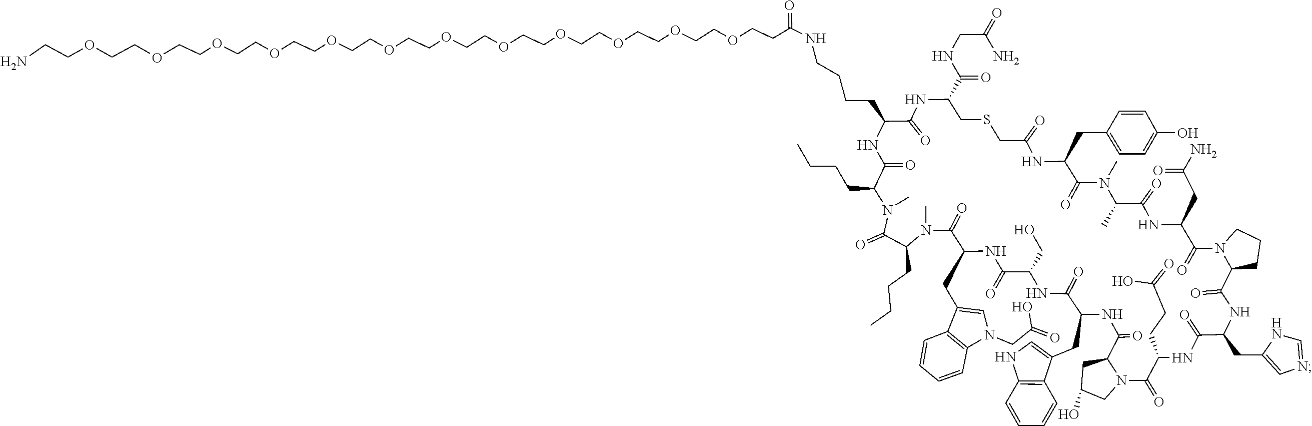 [SMILES: CCCC[C@H]1C(=O)N(C)[C@@H](CCCC)C(=O)N[C@@H](CCCCNC(=O)CCOCCOCCOCCOCCOCCOCCOCCOCCOCCOCCOCCOCCN)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](Cc2cnc[nH]2)C(=O)N[C@@H](CCC(=O)O)C(=O)N2C[C@H](O)C[C@H]2C(=O)N[C@@H](Cc2c[nH]c3ccccc23)C(=O)N[C@@H](CO)C(=O)N[C@@H](Cc2cn(CC(=O)O)c3ccccc23)C(=O)N1C]

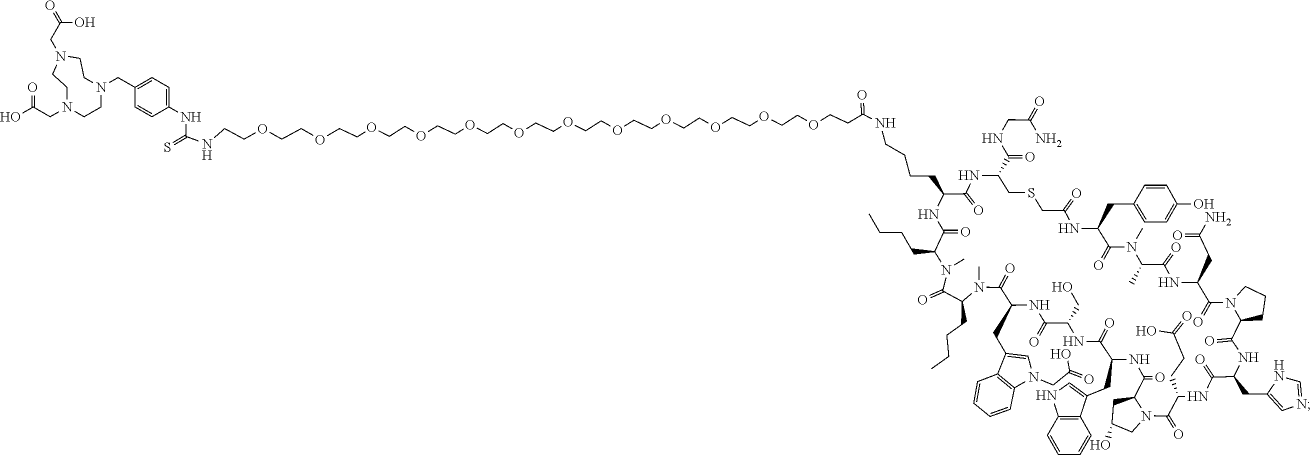 [SMILES: CCCC[C@H]1C(=O)N(C)[C@@H](CCCC)C(=O)N[C@@H](CCCCNC(=O)CCOCCOCCOCCOCCOCCOCCOCCOCCOCCOCCOCCOCCNC(=S)Nc2ccc(CN3CCN(CC(=O)O)CCN(CC(=O)O)CC3)cc2)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](Cc2cnc[nH]2)C(=O)N[C@@H](CCC(=O)O)C(=O)N2C[C@H](O)C[C@H]2C(=O)N[C@@H](Cc2c[nH]c3ccccc23)C(=O)N[C@@H](CO)C(=O)N[C@@H](Cc2cn(CC(=O)O)c3ccccc23)C(=O)N1C]

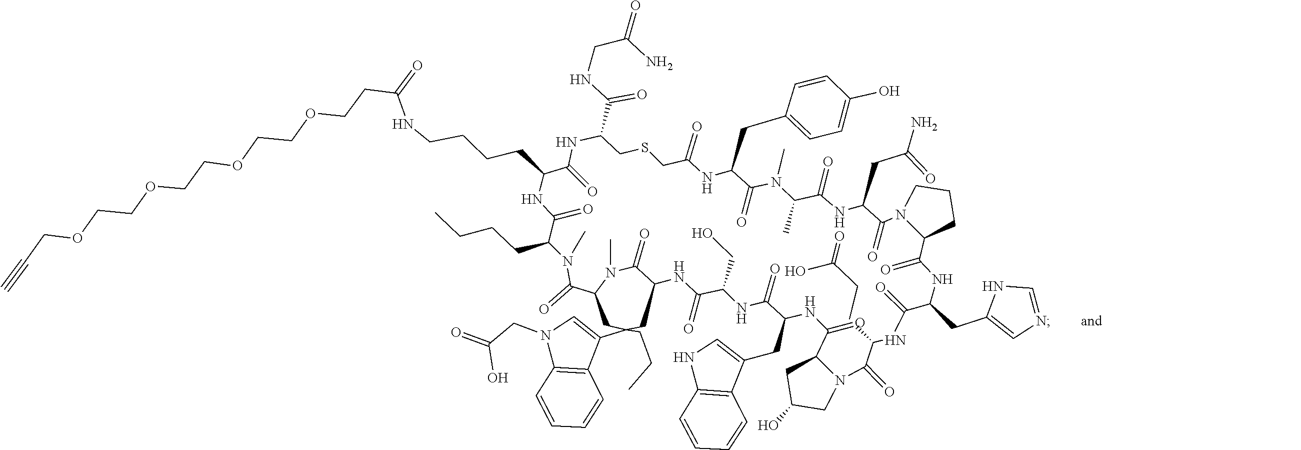 [SMILES: C#CCOCCOCCOCCOCCC(=O)NCCCC[C@@H]1NC(=O)[C@H](CCCC)N(C)C(=O)[C@@H]2CC(CCC)(c3cn(CC(=O)O)c4ccccc34)C[C@H](NC(=O)[C@H](CO)NC(=O)[C@H](Cc3c[nH]c4ccccc34)NC(=O)[C@@H]3C[C@@H](O)CN3C(=O)[C@H](CCC(=O)O)NC(=O)[C@H](Cc3cnc[nH]3)NC(=O)[C@@H]3CCCN3C(=O)[C@H](C(C)C(N)=O)NC(=O)[C@H](C)N(C)C(=O)[C@H](Cc3ccc(O)cc3)NC(=O)CSC[C@@H](C(=O)NCC(N)=O)NC1=O)C(=O)N2C ; "and"]

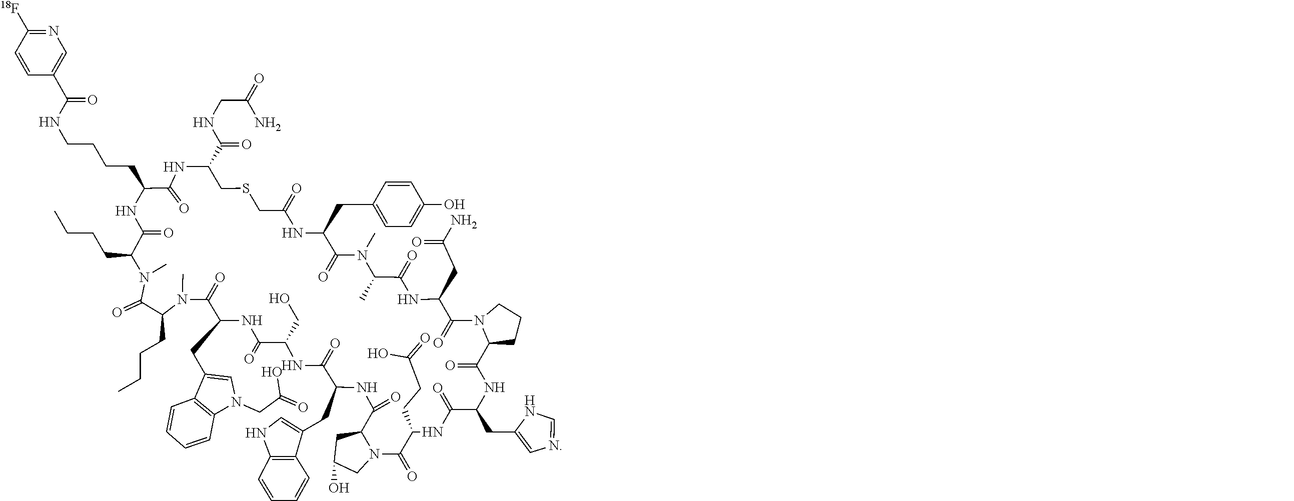 [SMILES: CCCC[C@H]1C(=O)N(C)[C@@H](CCCC)C(=O)N[C@@H](CCCCNC(=O)c2ccc([18F])nc2)C(=O)N[C@H](C(=O)NCC(N)=O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](Cc2cnc[nH]2)C(=O)N[C@@H](CCC(=O)O)C(=O)N2C[C@H](O)C[C@H]2C(=O)N[C@@H](Cc2c[nH]c3ccccc23)C(=O)N[C@@H](CO)C(=O)N[C@@H](Cc2cn(CC(=O)O)c3ccccc23)C(=O)N1C]

20. An imaging method for detecting Programmed Death Ligand 1 (PD-L1), the method comprising:
(a) providing an effective amount of an imaging agent of any of claims 1-19;
(b) contacting one or more cells or tissues with the imaging agent; and
(c) making an image to detect PD-L1.

21. The imaging method of claim 20, wherein contacting of the one or more cells or tissues with the imaging agent is performed in vitro, in vivo, or ex vivo.

22. The imaging method of claim 21, wherein contacting of the one or more cells or tissues with the imaging agent is performed in a subject.

23. The imaging method of claim 22, wherein the subject is a human, rat, mouse, cat, dog, horse, sheep, cow, monkey, avian, or amphibian.

24. The imaging method of claim 20, wherein detection of the PD-L1 occurs at about 60-120 minutes or less after administration of the imaging agent to the subject.

25. The imaging method of claim 20, wherein the imaging method is used to detect a cancer.

26. The imaging agent of claim 25, wherein the cancer is selected from the group consisting of a blastoma, a carcinoma, a glioma, a leukemia, a lymphoma, a melanoma, a myeloma, a sarcoma, head cancer, neck cancer, head and neck cancer, lung cancer, breast cancer, triple negative breast cancer, prostate cancer, colorectal cancer, esophageal cancer, stomach cancer, leukemia/lymphoma, uterine cancer, skin cancer, endocrine cancer, urinary cancer, pancreatic cancer, gastrointestinal cancer, ovarian cancer, cervical cancer, renal cancer, bladder cancer, brain cancer, adenoma, and a metastatic cancer.

27. The imaging method of claim 20, wherein the imaging method is used to detect a solid tumor.

28. The imaging method of claim 27, wherein the solid tumor is in an organ selected from the group consisting of brain, colon, breast, prostate, liver, kidney, lung, esophagus, head and neck, ovary, cervix, stomach, rectum, bladder, uterus, testes, and pancreas.

29. The imaging method of claim 20, wherein the imaging method is used to detect an infection.

30. The imaging method of claim 29, wherein the infection is a microbial infection.

31. The imaging method of claim 30, wherein the microbial infection is selected from the group consisting of an infection due to one or more microorganisms selected from the group consisting of Mycobacterium tuberculosis, E. coli, Klebsiella sp., Enterobacter sp., Proteus sp., Serratia marcescens, Pseudomonas aeruginosa, Staphylococcus spp., including S. aureus and coag.-negative Staphylococcus, Enterococcus sp., Streptococcus pneumoniae, Haemophilus influenzae, Bacteroides spp., Acinetobacter spp., Helicobacter spp., Candida sp., methicillin-resistant Staphylococcus aureus (MRSA) and vancomycin-resistant Enterococcus faecalis (VRE).

32. The imaging method of claim 20, wherein the imaging method is used to detect inflammation.

33. The imaging agent of claim 32, when in the inflammation is related to a disorder selected from the group consisting of asthma, an autoimmune disease, an autoinflammatory disease, Celiac disease, diverticulitis, glomerulonephritis, hidradenitis suppurativa, a hypersensitivity, an inflammatory bowel disease, interstitial cystitis, otitis, pelvic inflammatory disease, reperfusion injury, rheumatic fever, rheumatoid arthritis, sarcoidosis, transplant rejection, lupus, systemic lupus erythematosus, and vasculitis.

34. The imaging method of claim 33, wherein the inflammation is caused by rheumatoid arthritis or systemic lupus erythematosus.

35. The imaging method of claim 20, wherein the imaging method is used to detect one or more immune cells in a tumor.

36. The imaging method of claim 20, wherein the imaging method is used to detect systemic distribution of immune cells in the tumor or in a subject.

37. The imaging method of claim 20, wherein the imaging method is used to detect an immune cell response to an infectious disease.

38. The imaging method of claim 20, wherein the imaging method is used to detect an immune cell response in a tumor or in normal tissue response to an inflammatory disease.

39. The imaging method of claim 20, wherein the imaging method detects PD-L1 expression levels in the subject.

40. The imaging method of claim 20, wherein the imaging method measures an occupancy of PD-L1 at a tumor site or in normal tissue of the subject.

41. A kit for detecting Programmed Death Ligand 1 (PD-L1), the kit comprising the imaging agent of any of claims 1-19.
